(19) United States
(12) Patent Application Publication
(10) Pub. No.: US 2024/0036050 A1
(43) Pub. Date: Feb. 1, 2024

(54) Methods Of Detecting And Treating Hepatocellular Carcinoma

(71) Applicants: The United States Government As Represented By The Department Of Veterans Affairs, Washington, DC (US); THE ADMINISTRATORS OF THE TULANE EDUCATIONAL FUND, New Orleans, LA (US)

(72) Inventors: Srikanta Dash, New Orleans, LA (US); Yucel Aydin, New Orleans, LA (US); Ali Riza Koksal, New Orleans, LA (US)

(21) Appl. No.: 18/356,537

(22) Filed: Jul. 21, 2023

Related U.S. Application Data
(60) Provisional application No. 63/391,000, filed on Jul. 21, 2022.

(51) Int. Cl. G01N 33/574; A61K 45/00

(52) U.S. Cl. CPC G01N 33/57438; A61K 45/00; G01N 2800/085; G01N 2333/46; G01N 2800/52

(57) ABSTRACT

Disclosed are methods of treating a subject having hepatocellular carcinoma (HCC) comprising administering an HCC therapeutic to a subject identified in need thereof, wherein the subject was identified as being in need of thereof by determining the subject had an increased expression level of glypican 3 (GPC3) and/or an increased number of GPC3-enriched exosomes/increase level of exosome-derived GPC3 (eGPC3) as compared to a control. Disclosed are methods of diagnosing and treating a subject comprising detecting whether GPC3 expression is increased in the subject and/or GPC3-enriched exosomes are increased; diagnosing the subject with HCC when the presence of elevated GPC3 and/or an increased number of GPC3-enriched exosomes is detected; and administering a therapeutically effective amount of an HCC therapeutic to the subject. Disclosed are methods of detecting HCC in a subject comprising determining the level of GPC3 positive exosomes and/or the expression level of exosome derived GPC3 in a sample obtained from a subject and comparing the level of GPC3 positive exosomes and/or the expression level of exosome derived GPC3 from the subject to a control, wherein an increase in the level of GPC3 positive exosomes and/or an increase in the expression level of exosome derived GPC3 in the subject, as compared to a control, is a detection of HCC in the subject.

### CROSS-REFERENCE TO RELATED APPLICATIONS

This application claims the benefit of U.S. Provisional Patent Application No. 63/391,000, filed Jul. 21, 2022, each of which is incorporated by reference herein in its entirety.

### STATEMENT REGARDING FEDERALLY SPONSORED RESEARCH

This invention was made with government support under Grant Number 1101 BX004516-01A1 awarded by the BLRD, Veterans Affairs. The government has certain rights in this invention.

### BACKGROUND

Hepatocellular carcinoma (HCC) is the fifth most common cancer and the second leading cause of cancer-related mortality worldwide. Even though a great progress has been made in the treatment of HCC including curative resection or liver transplantation, tumor-directed therapies such as radiofrequency ablation, trans-arterial chemoembolization (TACE), radioembolization, as well as new systemic therapy beyond sorafenib, disease prognosis remains very poor. Recent HCC surveillance data from the United States show improved 5-year overall survival following treatment in HCC diagnosed in its early stages (44% versus 11%). Liver ultrasound (US) alone or combination with serum alpha-fetoprotein (AFP) every 6-months is the standard of care for HCC screening of patients with cirrhosis, although this approach lacks the sensitivity and specificity to reliably detect HCC at an early stage. During the last several years, numerous biomarkers have been introduced for surveillance and early detection of HCC in cirrhotic patients. The most promising targets include glypican 3 (GPC3), Osteopontin, Golgi protein-73 as well as several microRNAs and non-coding RNAs candidates. However, these candidates have yet to establish superiority to routine liver US and serum AFP. In addition, the molecular mechanisms, which link these biomarkers to HCC initiation, progression, and metastasis are unclear. Focusing on validated, tumor-derived candidate biomarkers should improve early-stage HCC diagnosis and access to the full range of treatment options to maximize patient outcomes.

Over the past several decades, chronic hepatitis C virus (HCV) infection has been the major risk factor for HCC.⁸ The rising incidence of metabolic syndrome, non-alcoholic fatty liver disease (NAFLD) is projected to soon become the predominant underlying HCC etiology. Although the number of HCV cured continues to rise, many of these patients still suffer from cirrhosis due to chronic inflammation, which may be further complicated by other risk factors including metabolic syndrome and excessive alcohol consumption. Noninvasive biomarkers that can predict the natural course of chronic liver disease, cirrhosis outcome, early prediction of premalignant disease and HCC are still urgently needed.

Autophagy is a lysosomal degradation process that provides cellular protection and increases the life span of many model organisms including mammals. During chronic liver disease hepatocytes, the major cell type in the liver, are exposed to a wide range of viral and non-viral insults that trigger stress response pathways. Autophagy is activated during the cellular stress response to reduce hepatic stress, restore cellular homeostasis, and promote cell viability. However, the capacity to sustain high autophagic activity declines with age and during chronic disease processes like HCC on a background of cirrhosis. Previous studies showed that hepatic adaptive response to multifaceted HCV microbial stress modulates the autophagy process leading to HCC development in cirrhosis. Furthermore, HCC initiation in cirrhosis is mechanistically linked to impaired autophagy. Chaperone-mediated autophagy and endosomal micro autophagy (exosome release) are specially activated as a compensatory, prosurvival mechanism under stress. The excessive release of non-degradable cellular cargoes/biomolecules through exosome release alleviates stress thereby promotes cell survival. Exosomes are double-layered extracellular vesicles of 50-150 nm diameter released by all mammalian cell during the normal physiological response to stress. Aberrant exosome secretion occurs during the cellular adaptive response to excessive stress caused tumor cell metabolism and proliferation. Exosomes are endocytic origin and are formed as intraluminal vesicles by inward budding of the late endosome or multivesicular bodies (MVBs). Many recent publications claim that exosomes play an important role in cell-to-cell communication and cancer metabolism. They carry numerous biomolecules, such as noncoding RNAs (ncRNAs), microRNAs and circular RNAs they regulation of gene transcription and translation. There is growing evidence suggesting that they play an important role in hepatobiliary cancers. It is hypothesized that HCC-derived exosomes carry specific proteins, nucleic acids in the form of mRNA, and small RNAs (miRNA or non-coding RNAs) that can be explored as a potential biomarker for early detection of HCC in cirrhosis.

Glypicans belong to a group of heparan-sulfate proteoglycans, a large family of plasma membrane-associated glycoproteins involved in endocytosis, lysosomal degradation, recycling, endosomal escape into cytosol, fusion into MVBs, and exosome release. GPC3 is involved in the endocytosis and degradation of cell surface receptors and plays an important role in HCC growth and differentiation by regulating activities of many signaling pathways. GPC3 and other proteoglycans play a role in ligand internalization, sorting, and degradation in the endosomes and MVBs. Exosomes express glypicans on their cellular surface. Recently, some researchers have reported that glypican exosomes can be used for the detection of pancreatic cancer and gastrointestinal cancers. Although GPC3 expression is increased in HCC and accurately differentiates HCC from the benign liver, clinical studies have produced controversial results on the utility of soluble serum GPC3 for detecting HCC.

Disclosed herein are methods of diagnosing and treating HCC in patients with cirrhosis by detecting or identifying subjects with increased levels of exosome-derived GPC3.

### BRIEF SUMMARY

Disclosed are methods of treating a subject having hepatocellular carcinoma (HCC) comprising administering an HCC therapeutic to a subject identified in need thereof, wherein the subject was identified as being in need of thereof by determining the subject had an increased number of GPC3-positive exosomes, and/or an increased expression level of glypican 3 (GPC3) and/or an increased level of exosome-derived GPC3 (eGPC3) as compared to a control.

Disclosed are methods of diagnosing and treating a subject comprising detecting whether GPC3-enriched exosomes are increased and/or GPC3 expression is increased in the subject and/or; diagnosing the subject with HCC when the presence of elevated GPC3 and/or an increased number of GPC3-enriched exosomes is detected; and administering a therapeutically effective amount of an HCC therapeutic to the subject.

Disclosed are methods of detecting HCC in a subject comprising determining the level of GPC3 positive exosomes and/or the expression level of exosome derived GPC3 in a sample obtained from a subject and comparing the level of GPC3 positive exosomes and/or the expression level of exosome derived GPC3 from the subject to a control, wherein an increase in the level of GPC3 positive exosomes and/or an increase in the expression level of exosome derived GPC3 in the subject, as compared to a control, is a detection of HCC in the subject.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1B show a Schematic diagram of immune magnetic bead binding assay used for isolation of exosomes and microvesicles. FIG. 1A shows 4.5 micron-diameter streptavidin magnetic beads were coated with biotin-labeled antibodies used to capture exosomes and microvesicles. A fluorescence (FITC or PE) labeled antibody was used for the detection of exosomes and microvesicles by flow cytometry. FIG. 1B shows the steps used to purify exosomes or microvesicles in their native stage.

FIGS. 2A-2D: Show the specificity of the immune-magnetic bead method used to isolate exosomes and microparticles. FIG. 2A shows gating strategy of streptavidin-labeled magnetic beads used by flow cytometry using PE-labeled biotin or FITC-labeled biotin. The forward scatter (FSC), and side scatters (SSC) data were used to gate for a single population of beads for analysis, avoiding clumped beads. FIG. 2B shows the percentage of magnetic beads conjugation with streptavidin using FITC-Biotin and PE-Biotin. FIG. 2C shows validation of immune-magnetic affinity isolation methods using extracellular vesicles isolated from Huh 7 cell culture. A very high percentage of exosomes were detected in human serum using CD63 antibody-coated magnetic beads. A very high rate of ARF6 positive microvesicles was seen using Annexin A1 coated beads. FIG. 2D shows reproducibility of the assay separating exosomes from microvesicles.

FIGS. 3A-3B: Show immune-magnetic affinity isolation detect glypican-3 in HCC cell culture-derived exosomes, not in the microvesicles. FIG. 3A shows the detection of exosomes and microvesicles in the extracellular vesicles isolated from HCC cell culture. First, EVs were isolated from HCC cell culture by ExoQuick (total exosome isolation reagent) precipitation. Second, exosomes and microvesicles were selected from total EVs using CD63 or Annexin 1 antibodies or Heparin conjugated magnetic beads. Immuno-affinity capture exosomes or microvesicles were stained with Alexa Fluor 488 labeled glypican-3. Approximately 53.4% of CD63 conjugated magnetic beads show positive for glypican-3 as compared to 0.1% of heparin beads and 0.9% of Annexin A1 conjugated beads. About 92.8% of CD63 beads show positive for CD81, as compared to 89.5% of heparin beads and 45.7% of Annexin A1 beads. FIG. 3B shows a Western blot analysis shows the distribution of GPC3 in the total non-selected and selected EVs.

FIGS. 4A-4E: Show immuno-magnetic bead assay detects glypican-3 in HCC exosomes, not exosomes secreted by cholangiocarcinoma (HuCCA) or human embryonic kidney cells (HEK293). FIG. 4A shows GPC3 detection in exosomes isolated from two HCC cell lines. GPC3 was not detected in exosomes isolated from non-HCC cell lines (HuCCA or HEK293). FIG. 4B shows relative distribution of glypican-3 and CD81 among HCC and non-HCC cultures. FIG. 4C shows the concentration of EVs isolated from hepatic and non-hepatic culture by NanoSight. FIG. 4D shows the average size of EVs isolated from hepatic and non-hepatic cell cultures. FIG. 4E shows nanoparticle tracking analysis showing the actual view of particle size and concentration by Nano sight.

FIGS. 5A-5D: Show impaired autophagy promotes GPC3 accumulation in exosomes. CD63-GFP stable cell line was treated with increasing concentrations of Torin1 or Bafilomycin A1 for 72 hours. FIG. 5A demonstrates the expression of CD63-GFP in Torin1 treated and Bafilomycin A1 treated by fluorescence microscopy. FIG. 5B shows the quantification of CD63-GFP expression in Torin 1 and Bafilomycin A1 treated culture by Image J software. FIG. 5C shows the expression of CD63 and glypican-3 in Torin 1 and Bafilomycin A1 treated cells by fluorescence microscopy. FIG. 5D shows a Western blot analysis shows autophagy induction by Torin 1 leads to GPC3 and CD63 degradation.

FIGS. 6A-6C: Show the impact of autophagy on exosome and microvesicle production in HCC cell culture. FIG. 6A shows Huh 7 cells treated with autophagy inducer (Torin 1) and autophagy inhibitor (Bafilomycin A1). Total EVs were isolated from Huh7 cells by ExoQuick (Invitrogen). Nanoparticle tracking analysis showing the actual view of concentration and size of EV preparation. FIG. 6B shows EV concentration in Huh7 cells treated with Torin 1 and Bafilomycin A1. FIG. 6C shows the impact of autophagy on exosome and microvesicle production determined by the immune-magnetic affinity isolation method. Autophagy induction decreased the production of exosomes, whereas autophagy inhibition increased exosome production. Autophagy modulation has a similar effect on microvesicle production. Autophagy induction decreased GPC3 expression.

FIGS. 7A-7D: Show Glypican 3 detection in exosomes and microvesicles using serum samples from patients with HCC. Validation studies of glypican3 GPC3 enriched in exosomes, not in the microvesicles of HCC patients. Immuno-magnetic isolation protocol for the detection of GPC3 using 10 ml of serum samples from healthy liver cirrhosis without and with HCC patients. FIG. 7A shows a nanosight view of EVs isolated from healthy control, cirrhosis, and HCC. FIG. 7B shows the concentration and size of EVs isolated from 10-microliters of serum from healthy control, cirrhosis without, and with HCC. FIG. 7C shows the GPC3 enrichment in exosomes and microvesicles isolated from serum samples. Immunoaffinity isolation of microvesicles shows a negative expression of GPC3. FIG. 7D shows CD81 detection in exosomes and microvesicles from healthy controls, liver cirrhosis, and HCC patients.

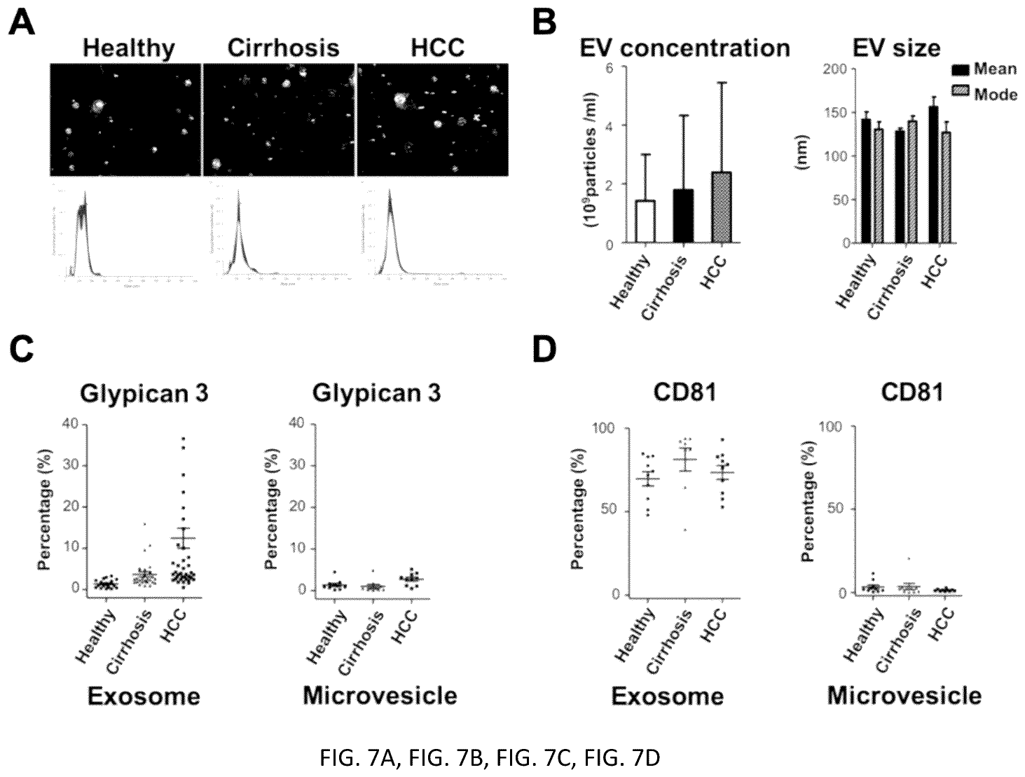

FIGS. 8A-8F: Show Immunohistochemical staining of p62 and glypican 3 (GPC3) in cirrhotic livers with HCC. FIG. 8A and FIG. 8D: Show histologic sections of HCC with adjacent liver cirrhosis (H&E, ×200). FIG. 8B and FIG. 8C: Show the expression of p62 in HCC and adjacent cirrhotic livers (various etiologies) with semiquantitative scoring for expression of the entire cohort. FIG. 8E and FIG. 8F: Show representative expression of GPC3 in HCC and adjacent cirrhotic livers (various etiologies) with semiquantitative scoring for expression of the entire cohort. Original image magnification was 200×. Data indicate mean±standard error of mean and analyzed by Student's t-test. ***P<0.001.

FIGS. 9A-9H: Show Association between p62, autophagy status, and glypican 3 (GPC3) expression in HCC cell lines and normal hepatocytes. FIG. 9A shows a panel of HCC cell lines and primary human hepatocytes (PHH) immunostained for p62. Scale bars=m. FIG. 9B shows relative optical density (OD) of p62 immunostaining per representative view field. The results are expressed as the mean±standard deviation (SD). ***P<0.001. For each cell line 10 view fields were quantified. FIG. 9C shows a representative Western blot of whole cell extracts for p62 and loading control. FIG. 9D shows the images of DQ-Red BSA uptake and processing in autophagy-deficient (Huh-7, Huh-7PX, and Huh-7.5) and an autophagy-competent (HLE) HCC cell lines±Torin1 treatment (200 nM). Scale bars=20 m. FIG. 9E shows a representative Western blot analysis of p62 degradation after Torin1 treatment in autophagy-deficient (Huh-7, Huh-7PX, and Huh-7.5) and an autophagy-competent (HLE) HCC cell lines. FIG. 9F shows GPC3 and glypican 1 (GPC1) immunostaining in autophagy-deficient (Huh-7, Huh-7PX, and Huh-7.5) and an autophagy-competent (HLE) HCC cell lines. Scale bars=20 m. FIG. 9G shows relative OD of GPC3 and GPC1 immunostaining per representative view field. Data indicate mean±SD. ***P<0.001. FIG. 9H shows a representative Western blot analysis showing the expression of GPC3 and GPC1 in autophagy-deficient (Huh-7, Huh-7PX, and Huh-7.5) and an autophagy-competent (HLE) HCC cell lines. The data are resulted in three independent experiments.

FIGS. 10A-10D: Show characterization of exosomes released from an autophagy-deficient and an autophagy-competent HCC cell line. FIG. 10A shows cryogenic transmission electron microscopy (cryoTEM) images of exosomes purified from culture supernatants of autophagy-deficient (Huh-7.5) and autophagy-competent (HLE) HCC lines. Left panel (low magnification). Right panel (high magnification). FIG. 10B Top panel: shows a representative image of exosome Brownian motion in liquid phase using Nanoparticle tracking analysis (NTA). Bottom panel: Size distribution profile of exosomes with mean and mode of the population diameter±standard error. FIG. 10C shows exosome concentration in the culture supernatant quantified by NTA. The results are expressed as the mean±standard deviation and analyzed by Student's t-test. *P<0.05. FIG. 10D shows a representative Western blot for TSG101 and CD9 in cell lysates and exosomes isolated from autophagy-deficient Huh-7.5 and autophagy-competent HLE cell cultures. All experiments were performed in triplicate.

FIGS. 11A-11F: Show Glypican 3 (GPC3)-enriched exosomes are release exclusively from autophagy-deficient hepatocellular carcinoma (HCC) cells. FIG. 11A shows a cell compartment breakdown of 129 proteins identified by proteomic analysis of exosomes isolated from autophagy-deficient Huh-7.5. FIG. 11B shows a representative immunogold-transmission electron microscopy images of exosomes labeled against glypican 1 (GPC1) and GPC3 after isolation from autophagy-deficient (Huh-7.5) and autophagy-competent (HLE) HCC lines. FIG. 11C shows fluorescence histograms of bead-captured exosomes from Huh-7.5 and HLE stained for GPC1. FIG. 11D shows fluorescence histograms of bead-captured exosomes from Huh-7.5 and HLE stained for GPC3. FIG. 11E shows background fluorescence of bead-captured exosomes with primary antibody omitted. FIG. 11F shows a representative Western blot analysis of GPC1 and GPC3 in purified exosomes isolated from Huh-7.5 and HLE cultures. All experiments were in triplicate.

FIGS. 12A-12E: Show characterization of circulating exosomes among non-cirrhotics and cirrhotics with and without hepatocellular carcinoma (HCC). FIG. 12A shows a representative nanoparticle tracking analysis (NTA) images and distribution plots from exosomes isolated from the serum. Size distribution profile of exosomes with mean and mode of the population diameter±standard error. FIG. 12B shows serum exosome concentrations determined by NTA (n=25 per sample group). Data indicate mean±standard deviation (SD) and analyzed by Student's t-test. P values were displayed as ^{ns} P>0.05, *P<0.05, ***P<0.001. FIG. 12C shows the size distribution of isolated exosomes among sample groups. The results are expressed as the mean±SD and analyzed by Student's t-test. FIG. 12D shows the concentration of exosome-derived CD63 (eCD63). Data indicate mean±standard error of mean and analyzed by Student's t-test. P values were displayed as ^{ns} P>0.05, *P<0.05, ***P<0.001. FIG. 12E shows a receiver operating characteristic (ROC) curves for eCD63 in distinguishing HCC from non-cirrhotic controls (solid blue), HCC from cirrhosis alone (dashed green), and cirrhosis from non-cirrhotic controls (solid red).

FIGS. 13A-13D: Shows exosome-derived glypican 3 (eGPC3) and p62 (ep62) levels as prognostic performance in detecting hepatocellular carcinoma (HCC). FIG. 13A shows concentration of eGPC3 among cohort disease groups (n=25 in healthy, cirrhosis, and HCC. n=10 in other tumors). P values were displayed as ^{ns} P>0.05 and ***P<0.001. FIG. 13B shows receiver operating characteristic (ROC) curves of eGPC3 in HCC compared to other disease states with AFP ROC as reference. P values were displayed as **P<0.01 and ***P<0.001. FIG. 13C shows ep62 concentrations among cohort groups. P values were displayed as ^{ns} P>0.05. FIG. 13D shows ROC curve analysis for ep62 level compared HCC to other disease states. Data indicate mean±standard error of mean and analyzed by Student's t-test. P values were displayed as ^{ns} P>0.05.

FIGS. 14A-14H: Show changes in exosome-derived glypican 3 (eGPC3) and CD9 (eCD9) in response to tumor-directed therapy for hepatocellular carcinoma (HCC). FIG. 14A shows affinity capture ELISA optical density (OD) for eGPC3 before and after doxorubicin-eluting bead transarterial chemoembolization (DEB-TACE) in patients responding to therapy. FIG. 14B shows OD for eCD9 in patients responding to treatment. FIG. 14C shows serum AFP levels in treatment responsive patients. FIG. 14D shows OD for eGPC3 in patients who did not respond to DEB-TACE. FIG. 14E shows OD for eCD9 in treatment non-responders. FIG. 14F shows serum AFP levels in treatment non-responders. FIG. 14G shows receiver operating characteristic (ROC) curve plots for decrease in eCD9 content compared to AFP decrease post-treatment for response to treatment. FIG. 14H shows ROC curve plot of in eGPC3 compared to increase in AFP for non-response to treatment. Data indicate mean±standard error of mean and analyzed by paired t-test. P values were displayed as ^{ns} P>0.05, *P<0.05, **P<0.01, ***P<0.001.

FIGS. 15A-15D: Shows exosome capture by affinity ELISA for multiplex surface analysis of exosome-associated molecules. FIG. 15A shows a schematic illustration of the affinity ELISA capture and analytic approach. FIG. 15B shows a representative affinity ELISA plate of multiplex analysis of HSC70, glypican 3, and CD9 in exosomes bound from the microplate. FIG. 15C shows concentration-dependent detection of HSC70, CD9, and glypican 3 on exosome surface. FIG. 15D shows a representative picture of affinity ELISA for CD9 and glypican 3 detection on HCC exosomes isolated from the serum of HCC patients before and after chemotherapy.

FIG. 16 shows the effect of exosome isolation/precipitation before the immune-magnetic affinity assay and serum volume on exosomal CD81 expression. CD63 coupled beads and serum samples obtained from healthy individuals were used in this analysis.

FIGS. 17A-17B show an overview of the experimental workflow of F-NTA assay to quantify HCC exosomes in their native state. (FIG. 17A). Shows antibody selection to immunocapture HCC exosomes. PE-labeled AFP and GPC3 antibodies are specific to HCC-derived exosomes used alone or in combination. (FIG. 17B). Brief presentation of clinical assay used to capture highly purified HCC exosomes for F-NTA quantification. 10 μl of human serum was diluted in 100 μl of PBS with 1% BSA. Antibody incubation was performed overnight at 4° C. with shaking. The next day, the sample was diluted in 1 ml of ultrapure-filtered distilled water and passed through the Sephadex column. Samples were further diluted 1:10 in ultrapure water and analyzed by NTA in LSM and FM.

FIGS. 18A-18D show optimization of F-NTA measurement using synthetic fluorescent and non-fluorescence beads. (FIG. 18A). Shows video images of unlabeled and labeled fluorescence synthetic beads in suspension examined using NTA in light scattered mode (LSM) and fluorescence mode (FM). Only fluorescence-labeled synthetic beads were detected in both LSM and FM (FIG. 18B). Show the sensitivity and specificity of F-NTA analysis using synthetic beads. A snapshot of video images of particles in motion using NTA analysis of synthetic beads in LSM and FM. A fixed amount of unlabeled synthetic beads were spiked with different concentrations of fluorescence-labeled synthetic beads. (FIG. 18C). Shows particle size distribution of synthetic beads analyzed by NTA. (FIG. 18D). Show quantification of synthetic fluorescence beads by NTA. The detection range of F-NTA is shown.

FIGS. 19A-19D show recovery and quality assessment of immunocapture-HCC exosomes purified by size exclusion column purification for NTA analysis. Immunocaptured HCC exosomes were diluted in 1 ml of ultrapure water and passed through a gel filtration column to separate free labeled antibodies. (FIG. 19A). Size and concentration measurement EVs isolated by various columns by NTA. (FIG. 19B). Video images of EVs in motion captured via light scatter model. (FIG. 19C) Recovery of antibody-labeled exosomes by various columns assessed by concentration and size measurement by NTA.

FIGS. 20A-20C shows purification of EVs by size exclusion chromatography preserves immunoreactivity assessed by F-NTA. (FIG. 20A). Shows effectiveness of purification after using lipophilic membrane dye (Cell Mask™ Plasma Membrane Stain) for staining EV's. According to percentages of the positive stained vesicles, most of the EV's are membranous. (FIG. 20B). A pie chart shows the EV population diversity of exosome and microvesicle after immunocaptured using PE-labeled CD9 antibody and PE-labeled ARF6 antibody from cell culture, and clinical samples (normal serum, cirrhosis with or without HCC). As predicted, the F-NTA analysis shows exosome release increased in chronic liver disease as compared to only among healthy individuals. A large percentage of EVs remain unlabeled. (FIG. 20C). Show clinical application of F-NTA to quantify liver-derived EVs using PE-labeled antibodies to ASGPR1. The percentage of liver-derived vesicles was lower in cirrhosis and HCC groups than the healthy controls.

FIGS. 21A-21C show an assay calibration using exosome purified from Huh7 culture. Approximately 1×10⁷ Huh 7 cells were cultured in a 10-cm tissue culture plate. Media were collected after 72 hours and exosomes were precipitated from 1-ml cell-free supernatants by precipitation. Exosomes were recovered after centrifugation. The exosome pellet was resuspended in 100 μl of PBS with 1% BSA and incubated with PE-labeled GCP3, AFP, and CD9 overnight. The next day, samples were diluted in 1 ml water and passed through a column. EVs collected from flow-through were diluted in water (1:10) and immediately analyzed by NTA using LSM and FM. (FIG. 21A). Images of PE-conjugated CD9 antibody labeled EVs were captured from Huh7 and HuCCA-1 culture and measured by NTA in LSM and FM. (FIG. 21B). Representative image of PE-labeled GPC3 antibody-labeled HCC exosomes captured from HuCCA-1 and Huh7 cells and measured by NTA in LSM and FM. (FIG. 21C). Total exosomes and HCC exosomes are released per tumor cell. The total exosome release from HuCCA-1 cells is more than from Huh7 cells. HuCCA-1 cells release a minimum number of HCC exosomes as compared to Huh7 cells.

FIGS. 22A-22D show quantification of GPC3 positive HCC exosomes using F-NTA. Ten microliters of serum samples were diluted in 100 μl of PBS with 1% BSA. Antibody incubation was performed at 4° C. overnight. The next day samples were diluted in 1 ml of ultrapure water and passed through a gel-filtration column. Samples were further diluted (1:10) in 1 ml water and analyzed by NTA using LSM and FM. (FIG. 22A). Representative video image of EVs analysis captured in LSM and FM of immunocaptured using PE-conjugated GPC3 antibody. (FIG. 22B). Shows the size (mean and mode) measured in LSM and FM. (FIG. 22C) Concentration measurement of immunocaptured EVs (particles/ml) using GPC3 antibody levels in normal, cirrhosis, and HCC serum detected using FM. The particle number obtained in FM was normalized with the particle number obtained in LSM for each sample. (FIG. 22D). ROC analysis shows the sensitivity and specificity values of GPC3 vesicles in normal, cirrhosis, and HCC.

FIGS. 23A-23D show quantification of AFP+ve HCC exosomes using F-NTA. Ten microliters of serum samples were diluted in 100 μl of PBS with 1% BSA. AFP antibody (1:100) incubation was performed at 4° C. overnight. The next day samples were diluted in 1 ml of ultrapure water and passed through a gel-filtration column. Samples were further diluted (1:10) in 1 ml water and analyzed by NTA. (FIG. 23A). Representative video image of EVs analysis captured in LSM and FM of immunocaptured using PE-conjugated AFP antibody. (FIG. 23B). Shows the size (mean and mode) measured in LSM and FM. (C). Particle size distribution of EVs analyzed in LSM and FM. (FIG. 23C) Quantification of immunocaptured EVs using AFP antibody in normal, cirrhosis and HCC serum detected using FM. The particle number obtained in FM mode was normalized with the particle number obtained in LSM for each sample. (FIG. 23D). ROC analysis shows the sensitivity and specificity values of AFP vesicles in normal, cirrhosis, and HCC.

FIGS. 24A-24D show quantification of AFP and GPC3 double-positive HCC exosome using the combination of two antibodies (AFP and GPC3). (FIG. 24A). Representative video image captured during EVs analysis in LSM and FM of immunocaptured using PE-conjugated AFP and GPC3 antibody combination. (FIG. 24B). Show the size (mean and mode) of immunocaptured EVs using two antibodies measured in LSM and FM. (FIG. 24C) Quantification of immunocaptured EVs using a combination of two antibodies (GPC3/AFP) in normal, cirrhosis and HCC serum detected using FM. The particle number obtained in FM was normalized with the particle number obtained in LSM for each sample. (FIG. 24D). ROC analysis shows the sensitivity and specificity values of HCC exosome quantification using the combination of AFP and GPC3 antibodies in normal, cirrhosis, and HCC.

FIGS. 25A-25F show quantification of HCC exosomes after immunocaptured by GPC3 alone, AFP alone, and a combination of two by another method (magnetic bead-based-flow cytometry). (FIG. 25A). Illustrate the assay design. Streptavidin-conjugated magnetic beads were attached to a biotinylated CD63 antibody. The next day, beads were incubated with 10 μl of the serum diluted in 100 μl PBS with 1% BSA overnight. The next day, beads were washed twice and then incubated with PE-conjugated antibodies (GPC3 alone, AFP alone, and combination) for one hour. Following this step, beads were washed and analyzed by a flow cytometer. (FIG. 25B). Percentage of exosomes reacted with GPC3 antibody by flow assay in normal healthy serum, cirrhosis, and cirrhosis with HCC. (FIG. 25C). Percentage of HCC exosome captured using AFP antibody between normal, cirrhosis, and cirrhosis with HCC. (FIG. 25D). Percentage of HCC exosome captured using a combination of AFP and GPC3 antibodies in normal, cirrhosis, and cirrhosis with HCC. (FIG. 25E). Representative flow cytometry data showing very high reactivity of using GPC3 and AFP antibody. (FIG. 25F). ROC analysis shows the sensitivity and specificity of immunomagnetic bead assay for HCC exosome quantification by magnetic-based affinity flow assay.

FIGS. 26A-26H show a statistical analysis determines the performance of HCC exosome quantification by F-NTA among MRI positive and negative serum samples of our patient cohort. The fluorescence-positive exosome number is normalized with the total number of EVs captured by light scatter mode in each sample. (FIG. 26A). Quantification of GPC3+ve exosomes in serums obtained from MRI positive and negative cases. (FIG. 26B). Quantification of HCC exosome using PE-labeled AFP antibodies in the same set of serum samples. (FIG. 26C). Quantification of HCC exosomes using a combination of PE-labeled GPC3 and PE-labeled AFP antibodies. (FIG. 26D). Comparison of MRI-positive and MRI-negative cirrhosis samples for serum AFP levels. (FIG. 26E). Comparison for GPC3+ve HCC-exosome concentration in AFP negative and AFP positive groups. The upper limit of AFP was considered as 20 ng/ml (FIG. 26F). The exosome size is comparable between the MRI-positive and negative groups. (FIG. 26G). Show correlation between HCC-exosome quantification with HCC size determined by MRI in the HCC cohort. (FIG. 26H). ROC analysis shows the sensitivity and specificity values of marker-positive exosome quantification and serum AFP levels for diagnosing HCC in liver cirrhosis.

FIGS. 27A and 27B show dose dependent titration of cell membrane dye shows Huh 7 cell culture-derived EV concentrations in Fluorescence Mode (FM) and Light Scatter Mode (LSM).

FIGS. 28A and 28B show PE-labeled fluorescent antibody validation studies in serums of patients with cirrhosis and HCC for quantification of HCC exosomes. (FIG. 28A). Shows normalized fluorescent particle concentrations in different dilutions of GP3-PE antibody. (FIG. 28B). Shows normalized fluorescent particle concentrations in different dilutions of AFP-PE antibody.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an exosome” includes a plurality of such exosomes, reference to “the exosome” is a reference to one or more exosomes and equivalents thereof known to those skilled in the art, and so forth.

By a “therapeutically effective amount” of a composition as provided herein is meant a sufficient amount of the composition to provide the desired therapeutic effect. The exact amount required will vary from subject to subject, depending on the species, age, and general condition of the subject, the severity of disease (or underlying genetic defect) that is being treated, the particular composition used, its mode of administration, and the like. Thus, it is not possible to specify an exact “therapeutically effective amount.” However, an appropriate “therapeutically effective amount” may be determined by one of ordinary skill in the art using only routine experimentation.

The term “therapeutic” refers to a composition that treats a disease. For example, the therapeutics disclosed herein are compositions that treat hepatocellular carcinoma.

By “treat” is meant to administer a therapeutic or composition of the invention to a subject, such as a human or other mammal (for example, an animal model), that has an increased susceptibility for developing hepatocellular carcinoma, or that has hepatocellular carcinoma, in order to prevent or delay a worsening of the effects of the disease or condition, or to partially or fully reverse the effects of the disease.

By “prevent” is meant to minimize the chance that a subject who has an increased susceptibility for developing hepatocellular carcinoma will end up with hepatocellular carcinoma.

The terms “patient,” “subject,” “individual,” and the like are used interchangeably herein, and refer to any animal, or cells thereof whether in vitro or in situ, amenable to the methods described herein. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

An “effective amount” of a compound is that amount of compound which is sufficient to provide a beneficial effect to the subject to which the compound is administered. The phrase “therapeutically effective amount”, as used herein, refers to an amount that is sufficient or effective to prevent or treat (delay or prevent the onset of, prevent the progression of, inhibit, decrease or reverse) a disease or condition, including alleviating symptoms of such diseases. An “effective amount” of a delivery vehicle is that amount sufficient to effectively bind or deliver a compound.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods

1. Methods of Treating

Disclosed are methods of treating a subject having hepatocellular carcinoma (HCC) comprising administering an HCC therapeutic to a subject identified in need thereof, wherein the subject was identified as being in need of thereof by determining the subject had an increased number of glypican 3 (GPC3) positive exosomes as compared to a control. In some aspects, an increased number of GPC3 positive exosomes can be the same as an increased level of GPC3 positive exosomes, thus these terms can be used interchangeably.

In some aspects, a control can be a sample run parallel to the sample obtained from the subject, wherein the control sample is from a healthy subject. In some aspects, a healthy subject does not have HCC. In some aspects, a healthy subject does not have any known illnesses. In some aspects, a control can be a subject without HCC. In some aspects, a control can be a subject without HCC. In some aspects, a control can be a subject with chronic liver disease or an autoimmune liver disease but without HCC.

In some aspects, the methods of treating further comprise wherein the subject was identified as being in need of thereof by determining the level of alpha-fetoprotein positive exosomes. In some aspects, the methods of treating further comprise wherein the subject was identified as being in need of thereof by determining the level of syntenin-1 positive exosomes. Syntenin-1 positive exosomes can be used as a pan-cancer marker, not just HCC specific marker. In some aspects, a combination of biomarkers can be detected/measured on exosomes. For example, in some aspects, a subject can be identified as being in need of thereof by determining one or more of the following: an increased number of GPC3 positive exosomes, an increased number of GPC3-enriched exosomes, an increased level of exosome-derived GPC3 (eGPC3), an increased level of alpha-fetoprotein positive exosomes, an increased level of syntenin-1 positive exosomes, wherein the increase is compared to a control.

In some aspects, the methods of treating further comprise isolating HCC-derived exosomes from a sample obtained from a subject. Thus, in some aspects, HCC-derived exosomes are isolated from serum.

In some aspects, the methods can further comprise determining whether the HCC-derived exosomes are enriched with GPC3. In some aspects, “enriched” means an increased number of molecules of GPC3 per exosome.

In some aspects, the methods further comprise a step of determining the levels of exosome-derived GPC3 (eGPC3). In some aspects, the levels of eGPC3 can be determined in biological sample obtained from a subject. For example, the biological sample can be serum.

Disclosed are methods of treating a subject having HCC comprising determining an increase in the number of GPC3 positive exosomes and/or an increase in the expression level of exosome derived GPC3 and/or an increase in the level of GPC3 on an exosome in a sample obtained from a subject and administering an HCC therapeutic to the subject. In some aspects, the increase in the number of GPC3 positive exosomes and/or the increase in expression level of exosome derived GPC3 is compared to a control. Any one of the controls described throughout can be used. In some aspects, a control can be a subject without HCC. In some aspects, a control can be a subject without HCC. In some aspects, a control can be a subject with chronic liver disease or an autoimmune liver disease but without HCC. Any of the disclosed methods of determining an increase in the level of GPC3 positive exosomes and/or the expression level of exosome derived GPC3 can be used.

In some aspects, prior to determining if a sample obtained from a subject has an increased number of GPC3 positive exosomes and/or an increased expression level of GPC3 (e.g. on the exosomes (GPC3-enriched exosomes)) and/or an increased level of eGPC3 as compared to a control, a further step of isolating GPC3-positive exosomes can be performed. In some aspects, GPC3-positive exosomes or GPC3-enriched exosomes can be isolated by any known technique, such as, but not limited to, an enzyme-linked immunosorbent assay (ELISA) or use of anti-GPC3 or a combination of anti-GPC3 and anti-CD63. For examples, exosomes can be isolated using anti-CD63 and then levels of GPC3 can then be measured/detected, thus determining the exosome-derived GPC3.

In some aspects, the sample obtained from a subject is a biological sample. In some aspects, the sample is serum, plasma, blood, cerebrospinal fluid, mucus, or urine.

In some aspects, the subject has cirrhosis.

In some aspects, the HCC therapeutic is liver transplantation, chemotherapy, immunotherapy, transcatheter arterial chemoembolization (TACE), Tecentriq, Avastin®, Doxorubicin Eluting Beads+ transarterial chemoembolization, Radiofrequency ablation, ethanol injection, transplantation, or Y90 radioembolization. In some aspects, the chemotherapy can be, but is not limited to, sorafenib, or doxorubicin. In some aspects, one or more HCC therapies can be administered to a subject.

In some aspects, a subject having an increased level of GPC3 positive exosomes and/or an increased number of GPC3-enriched exosomes and/or and increased level of eGPC3 is an increase compared to a control. In some aspects, a control can be non-cirrhotic controls, cirrhotic controls, non-HCC malignancy controls. In some aspects, the controls can be samples from subjects that have other diseases or disorders but do not have HCC. In some aspects, the control is a sample obtained from a subject having cirrhotic liver and not HCC. In some aspects, a control can be a sample from a healthy subject with no known illnesses. In some aspects, a control can be a subject without HCC. In some aspects, a control can be a subject without HCC. In some aspects, a control can be a subject with chronic liver disease or an autoimmune liver disease but without HCC.

In some aspects, the increase in the number of GPC3 positive exosomes and/or number of GPC3-enriched exosomes and/or levels of eGPC3 is at least a 2-fold increase over that of the control. In some aspects, the increase can be a 0.5, 1, 1.5, 2, 2.5, 3, 3.5, 4, 4.5, 5 or greater increase over that of the control. In some aspects, the increase in number of GPC3 positive exosomes and/or number of GPC3-enriched exosomes and/or levels of eGPC3 is at least 5, 10, 15, 20, 25, 30, 45, 40, 45, or 50% higher than that of the control.

Also disclosed are methods of determining if a subject is a responder to a HCC therapy. Disclosed herein are methods of determining if a subject is a responder to a HCC therapy comprising determining the level of GPC3 positive exosomes in the subject, administering an HCC therapeutic to a subject, and determining the level of GPC3 positive exosomes in the subject after administering the HCC therapeutic, wherein when the levels of GPC3 positive exosomes are decreased after administering the HCC therapeutic compared to prior to administering with the HCC therapeutic to the subject identifies a subject that is a responder to the HCC therapy.

2. Methods of Diagnosing

Disclosed are methods of identifying a subject with hepatocellular carcinoma (HCC) comprising: determining the number of glypican 3 (GPC3) positive exosomes in the subject and comparing the level of glypican 3 (GPC3) positive exosomes in the subject to a control, wherein when the subject has an increased level of glypican 3 (GPC3) positive exosomes as compared to a control identifies a subject with HCC. In some aspects, the term “number” or “level” can be used interchangeably, for example, determining the number of GPC3 positive exosomes can be the same as determining the level of GPC3 positive exosomes.

Disclosed are methods of diagnosing and treating a subject comprising detecting whether the number of GPC3 positive exosomes is increased, GPC3 expression is increased in the subject and/or GPC3-enriched exosomes are increased in the subject; diagnosing the subject with HCC when the presence of an GPC3 positive exosomes is increased or an increased number of GPC3-enriched exosomes or GPC3 expression is increased is detected; and administering a therapeutically effective amount of an HCC therapeutic to the subject.

In some aspects, the increased number of GPC3 positive exosomes and/or an increased number of GPC3-enriched exosomes is detected in a sample obtained from the subject. In some aspects, the increase can be detected using any known techniques in the art, particularly any of the detection methods disclosed herein.

In some aspects, the methods of treating further comprise wherein the subject was identified as being in need of thereof by determining the level of alpha-fetoprotein positive exosomes. In some aspects, the methods of treating further comprise wherein the subject was identified as being in need of thereof by determining the level of syntenin-1 positive exosomes. Syntenin-1 positive exosomes can be used as a pan-cancer marker, not just HCC specific marker. In some aspects, a combination of biomarkers can be detected/measured on exosomes. For example, in some aspects, a subject can be identified as being in need of thereof by determining one or more of the following: an increased number of glypican 3 (GPC3) positive exosomes, an increased number of GPC3-enriched exosomes, an increased level of exosome-derived GPC3 (eGPC3), an increased level of alpha-fetoprotein positive exosomes, an increased level of syntenin-1 positive exosomes, wherein the increase is compared to a control.

In some aspects, the sample obtained from a subject is a biological sample. In some aspects, the sample is serum, plasma, blood, cerebrospinal fluid, mucus, or urine

In some aspects, the increased GPC3 expression is exosome derived.

In some aspects, the HCC therapeutic is liver transplantation, chemotherapy, immunotherapy, transcatheter arterial chemoembolization (TACE), Tecentriq, Avastin®, Doxorubicin Eluting Beads+transarterial chemoembolization, Radiofrequecy ablation, ethanol injection, transplantation, or Y90 radioembolization. In some aspects, the chemotherapy can be, but is not limited to, sorafenib or doxorubicin. In some aspects, one or more HCC therapies can be administered to a subject.

In some aspects, a subject having increased GPC3 expression has an increase in GPC3 compared to a control.

In some aspects, the subject has cirrhosis.

In some aspects, a control can be a sample run parallel to the sample obtained from the subject, wherein the control sample is from a healthy subject. In some aspects, a healthy subject does not have HCC. In some aspects, a healthy subject does not have any known illnesses. In some aspects, the control can be a non-cirrhotic control, cirrhotic control, and/or individual with other non-HCC malignancies. In some aspects, the control can be a subject having cirrhotic liver and not HCC. In some aspects, a control can be a sample from a healthy subject with no known illnesses. In some aspects, a control can be a subject without HCC. In some aspects, a control can be a subject with chronic liver disease or an autoimmune liver disease but without HCC.

In some aspects, the increase in number of GPC3 positive exosomes and/or number of GPC3-enriched exosomes and/or levels of eGPC3 is at least a 2-fold increase over that of the control. In some aspects, the increase can be a 0.5, 1, 1.5, 2, 2.5, 3, 3.5, 4, 4.5, 5 or greater increase over that of the control. In some aspects, the increase in number of GPC3 positive exosomes and/or number of GPC3-enriched exosomes and/or levels of eGPC3 is at least 5, 10, 15, 20, 25, 30, 45, 40, 45, or 50% higher than that of the control.

In some aspects, the methods can further comprise determining an increase in p62 expression.

Disclosed are methods of diagnosing a subject as having HCC comprising detecting whether GPC3 expression and/or the expression level of exosome derived GPC3 is increased in the subject; and diagnosing the subject with HCC when presence of elevated GPC3 and/or elevated exosome derived GPC3 is detected.

In some aspects, the GPC3 expression and/or the expression level of exosome derived GPC3 is detected using immune-affinity flow cytometry.

3. Methods of Detecting HCC

Disclosed herein are methods of detecting Hepatocellular carcinoma (HCC) in a subject. In some aspects, HCC can be detected in a subject by measuring GPC3 positive exosomes and/or levels of exosome derived GPC3.

Disclosed are methods of detecting HCC in a subject comprising determining the number of GPC3 positive exosomes and/or the expression level of exosome derived GPC3 in a sample obtained from a subject and comparing the number of GPC3 positive exosomes and/or the expression level of exosome derived GPC3 from the subject to a control, wherein an increase in the number of GPC3 positive exosomes and/or an increase in the expression level of exosome derived GPC3 in the subject, as compared to a control, is a detection of HCC in the subject.

In some aspects, determining the number of GPC3 positive exosomes in a sample comprises contacting the sample obtained from the subject with an anti-CD63 antibody. This allows for capturing exosomes from the sample because anti-CD63 binds to exosomes. Once total exosomes are captured, the exosomes can be incubated with a GPC3 antibody. This allows for identifying or quantifying exosomes bound to the GPC3 antibody (e.g. GPC3 positive exosomes). In some aspects, the final step of determining the number of GPC3 positive exosomes in a sample comprises comparing the number of exosomes bound to the GPC3 antibody to the number of exosomes bound to the same GPC3 antibody in a control, wherein higher numbers of GPC3 positive exosomes in the sample compared to the control is an increase in the number of GPC3 positive exosomes.

In some aspects, identifying the GPC positive exosomes and/or exosome derived GPC3 bound to a GPC3 antibody can be through direct detection using a labeled GPC3 antibody or indirect detection using a secondary labeled antibody that binds the GPC3 antibody.

Disclosed are methods of detecting HCC in a subject comprising contacting a sample obtained from the subject with an anti-CD63 antibody to capture total exosomes from the sample; incubating the total exosomes with an GPC3 antibody; detecting or quantifying exosomes bound to the GPC3 antibody (e.g. GPC3 positive exosomes); determining the level of GPC3 positive exosomes in the sample and/or expression level of exosome derived GPC3 and comparing the level of GPC3 positive exosomes and/or expression level of exosome derived GPC3 from the sample obtained from the subject to a control, wherein an increase in the level of exosomes bound to the GPC3 antibody and/or an increase in the expression level of exosome derived GPC3 in the subject as compared to the control is a detection of HCC in the subject.

In some aspects, the methods of treating further comprise wherein the subject was identified as being in need of thereof by determining the level of alpha-fetoprotein positive exosomes. In some aspects, the methods of treating further comprise wherein the subject was identified as being in need of thereof by determining the level of syntenin-1 positive exosomes. Syntenin-1 positive exosomes can be used as a pan-cancer marker, not just HCC specific marker. In some aspects, a combination of biomarkers can be detected/measured on exosomes. For example, in some aspects, a subject can be identified as being in need of thereof by determining one or more of the following: an increased expression level of glypican 3 (GPC3), an increased number of GPC3-enriched exosomes, an increased level of exosome-derived GPC3 (eGPC3), an increased level of alpha-fetoprotein positive exosomes, an increased level of syntenin-1 positive exosomes, wherein the increase is compared to a control.

In some aspects, contacting a sample obtained from a subject with an anti-CD63 antibody will result in binding of the anti-CD63 antibody to total exosomes in the sample. In some aspects, the anti-CD63 antibody can be immobilized. For example, the anti-CD63 antibody can be on a plate or bead. In some aspects, the anti-CD63 antibody can be immobilized indirectly. Indirect immobilization of anti-CD63 to a plate or bead can also be used. For example, an anti-Ig (e.g. anti-IgG) antibody can be immobilized on a plate or bead and then the anti-CD63 antibody is bound to the anti-Ig. The binding of the anti-CD63 antibody to total exosomes results in capturing or immobilizing the exosomes in the sample. The exosomes can then be further evaluated while bound to the anti-CD63 antibody or the exosomes can be isolated from the anti-CD63 antibody for further examination/studies. In some aspects, the CD63 antibody used in the disclosed methods can be a CD63 binding fragment of the CD63 antibody. In some aspects, the CD63 antibody can actually be a scFv fragment of a CD63 antibody.

In some aspects, the sample obtained from a subject is a biological sample. In some aspects, the sample is serum, plasma, blood, cerebrospinal fluid, mucus, or urine.

In some aspects, the methods can further comprise a step of obtaining a sample from the subject prior to any of the steps that require a sample obtained from the subject.

In some aspects, exosomes can be isolated after capturing with an anti-CD63 antibody. In some aspects, simply using the anti-CD63 antibody to capture exosomes results in isolating the exosomes because the remaining portions of the sample can be washed away. In some aspects, the exosomes can be removed from the anti-CD63 and thus are further isolated. Common techniques known in the art can be used to separate the exosomes from the anti-CD63 antibodies.

In some aspects, the GPC3 antibody used in the disclosed methods can be a GPC3 binding fragment of the GPC3 antibody. In some aspects, the GPC3 antibody can actually be a scFv fragment of a GPC3 antibody

In some aspects, the disclosed methods do not involve the use of detergent in any of the steps. In some aspects, the presence of a detergent in any of the steps, particularly the detection steps (e.g. determining steps), can result in negative results due to disrupting the exosomes.

The disclosed methods can also be used to distinguish patients with HCC from non-cirrhotic controls, cirrhotic controls, and/or individuals with other non-HCC malignancies. For example, a sample obtained from a subject having HCC can have an increase in the level of exosomes bound to the GPC3 antibody and/or an increase in the expression level of exosome derived GPC3 as compared to a control such as non-cirrhotic controls, cirrhotic controls, non-HCC malignancy controls. In some aspects, the controls can have other diseases or disorders but do not have HCC.

In some aspects of any of the disclosed methods, the analysis of GPC3 identifies exosomes, not microvesicles.

4. Methods of Screening

Disclosed are methods of screening comprising detecting the presence of GPC3 positive exosomes in a sample from a subject having HCC; adding a therapeutic to the sample; detecting the presence of GPC3 positive exosomes in the sample after incubating with the therapeutic; determining the therapeutic treats HCC when a decrease in GPC3 positive exosomes are present in the sample after administering the therapeutic.

Disclosed are methods of screening a whether a subject having HCC is responsive to a therapeutic comprising detecting the presence of GPC3 positive exosomes in a sample from the subject; administering the therapeutic to the subject; detecting the presence of GPC3 positive exosomes in a sample taken after administering the therapeutic; determining whether the subject is responsive to the therapeutic, wherein a decrease in GPC3 positive exosomes in the sample taken after administering the therapeutic indicates the subject is responsive to the therapeutic.

5. Administration

The disclosed methods can include one or more of the types of administration disclosed herein.

In the methods described herein, administration or delivery of the therapeutics to a subject can be via a variety of mechanisms. For example, the therapeutic can be formulated as a pharmaceutical composition.

Pharmaceutical compositions can be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

### C. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising an HCC therapeutic and an anti-GPC3 antibody and/or anti-CD63 antibody.

### EXAMPLES

### A. Example 1: Impaired Autophagy Response in Hepatocellular Carcinomas Sequester Glypican-3 in Exosomes, not in the Microvesicles

Mammalian cells release various membrane-enclosed vesicles called extracellular vesicles. They are called exosomes, amphisomes, microvesicles, and apoptotic bodies (1). Exosomes are 30-150 nm diameter vesicles derived from the endosomal system. The fusion of multivesicular bodies with the cell membrane promotes exosome release (2). Amphisomes are hybrid organelles generated due to the fusion of endosomes with autophagosomes. The impaired lysosomal degradation could cause amphisome release. Microvesicles are usually 150-1000 nm in diameter derived directly budding from the plasma membrane (3). Apoptotic bodies have diameters of 500 nm to 2 000 nm (4). Pathological conditions such as chronic liver disease related to viral and non-viral etiologies promote EV production and release (5,6).

Autophagy is a lysosomal degradation process that provides cellular protection and adaptation to stress. Autophagy is a cellular mechanism implicated in energy production by degrading endogenous and exogenous materials delivered to the cytoplasm. It also plays a vital role in extracellular vesicle biogenesis and secretion (7). Autophagy and endocytosis are two cellular processes needed to degrade and recycle membrane receptors. They have a common endpoint that requires lysosomal degradation (8). These two cellular processes are essential for attenuating oncogenic cell signaling and cancer prevention. Autophagy is activated during the cellular stress response to reduce hepatic stress, restore cellular homeostasis, and promote cell viability. Extracellular vesicles release increased when autophagy was impaired to sustain hepatitis C virus replication (9). Impaired autophagy promotes EVs release by directly fusing autophagic vesicles to the membrane (10). The deficient autophagy process leads to HCC development in cirrhosis seen in human and mouse models. Autophagy loss due to Beclin 1 and ATG develop HCC in the mouse model.

The liver plays a central role in maintaining the EV concentration in the blood by acting as a natural scavenger by controlling their uptake and release. Extracellular vesicle (microvesicles and exosome) release increased in chronic liver disease. Peripheral EVs are quite heterogenous since many different tissues and cells in the body secrete EVs. Identifying HCC-specific EVs should improve the power of the biomarker. The GPC3 plays an essential role in hepatic endocytosis, lysosomal degradation, MVBs biogenesis, and exosome release (12-14). GPC3 is an HCC-specific tumor biomarker. Understanding the contribution of microvesicles and exosomes in GPC3 shedding due to impaired autophagy response in HCC is important,

The lack of a reliable method to detect biomolecules in EVs in the native state hinders the widespread clinical use of biomarkers (15,16). Affinity purification of exosomes using antibodies offers a solution (17-19). The Example herein optimized the method to select exosomes and microvesicles circulating in the peripheral blood. Antibodies against tetraspanin (CD63, CD81, CD9) were used in selecting exosomes. Antibodies against (Annexin A1 or ARF6) were used to determine extracellular vesicles. The results show the immunoaffinity assay nicely separates microvesicles and exosomes. GPC3 is preferentially enriched in HCC exosomes, not in microvesicles. Autophagy induction degrades GPC3, whereas the impaired autophagy response accumulates GPC3 and promotes its release through exosomes. Immune-affinity flow cytometry is a powerful method that accurately detects exosomal glypican-3 in cirrhotic patients with HCC using a small volume of serum samples.

1. Methods

i. Cell Culture and Reagents:

Huh7.5 cells were cultured in DMEM high glucose with 10% fetal calf serum (FBS), L-glutamine, 1 mM sodium pyruvate, and 0.1 mM non-essential amino acids with 1% penicillin and streptomycin. Huh-7.5 cells were transfected with CD63-GFP plasmid, and multiple stable cell lines expressing (Huh-7.5-CD63-GFP) were selected using G-418 (Sigma-Aldrich, St. Louis, MO) selection. Human cholangiocarcinoma cell line (HuCCA) and human embryonic kidney cell line (HEK 293) were purchased from ATCC. These two cell lines were cultured according to the instruction provided by the supplier and maintained with regular medium change every three days. Torin 1 was purchased from EMD Millipore, Burlington, MA (475991-10MG), and Bafilomycin A1 were purchased from Cell Signaling Technology, Danvers, MA (54645S). Exosome-free fetal bovine serum was purchased from ThermoFisher Waltham, MA (A2720803). Monoclonal rabbit and mouse antibodies were used as primary antibodies in western blot as well as fluorescent microscopy imaging. Anti-CD81 monoclonal rabbit—MA5-13548—and anti-glypican3 monoclonal rabbit—MA5-16368 purchased from ThermoFisher. Anti-ARF6 monoclonal rabbit—5740S—and anti-Beta actin monoclonal rabbit—4970S purchased from Cell signaling, Anti-Tsg 101 monoclonal mouse sc-7964 purchased from Santa Cruz Biotechnology.

ii. EV Purification from Cell Culture Supernatant:

Cells were cultured in exosome-depleted medium three days before the EV analysis. Briefly, cellular debris was removed from the culture supernatant by centrifugation at 2000 g for 30 minutes. Cleared supernatants were transferred to a new tube and mixed with 0.5 volumes of total EV isolation reagent from Invitrogen Carlsbad, CA (4478359). Tubes were incubated at 40 C overnight. The following day samples were centrifuged at 10,000 g for one hour at 40 C. Carefully removed supernatant, and EV pellet was resuspended in PBS and stored at −200 C. EVs concentration and size distribution were determined using NanoSight (Model NTA3300 with 532 nm green laser module, Malvern, Worcestershire, UK). Biotin anti-human CD63 antibody (Clone: H5C6) was purchased from BioLegend, San Diego, CA (353018). Biotin-conjugated Annexin A1 antibody was purchased from FabGennix Frisco, TX (ANXA1-BIOTIN). Biotin conjugated heparin was purchased from Sigma-Aldrich, Saint Louis, MO (B9806-10MG). Anti-glypican-3 (sc-390587 AF488), Anti-ARF6 (sc-7971 FITC), and Anti-CD81 (sc-166029 PE) were purchased from Santa Cruz Biotechnology, Santa Cruz, CA.

iii. Immuno Magnetic Bead-Based Affinity Flow Cytometry:

Streptavidin-coated magnetic beads were purchased from Invitrogen Carlsbad, CA (10608D). Beads were prewashed in phosphate buffer saline (PBS) and then incubated with biotinylated antibodies for one hour. Antibodies to CD63 or Annexin A1 were used at a proportion of 4 μg/5 mg of beads, or biotinylated heparin was used at 100 μg/1 mg of beads. Antibody conjugated beads were washed twice using PBS with 1% BSA to block the nonspecific bindings sites. The beads were incubated with 100 μl of cell culture-derived EVs overnight at 40° C. The following protocol was used for immuno-magnetic separation of EVs in human serum. Serum samples were centrifuged briefly at 2000 rpm for 5 minutes to remove debris. Ten microliters of serum were diluted in 100 μl PBS and used directly incubated with an immunomagnetic bead to capture EVs in the native form. The next day, the EV-bead complex was kept in a magnetic stand and washed three times using PBS with 1% BSA. Following this step, the EV-bead complex was incubated with fluorescence-labeled antibodies diluted in 100 μl PBS 1% BSA for one hour at room temperature. Beads were washed in PBS 1% BSA and then analyzed on a Becton-Dickinson flow cytometer (BD FACS Celesta) using BD FACSDiva Software 6.0, San Jose, CA. Advanced flow cytometry analyses and graph productions were performed using Flowing software version 2.5.1 developed by Turku bioscience (Turku, Finland)

iv. Serum Cohort Study:

HCC Serum samples used in this study were obtained. HCC was diagnosed by biopsy or met the Liver Imaging Reporting and Data System (LI-RADS). This study was also conducted in accordance with the Declaration of Helsinki. Serum samples were obtained from cirrhotic patients. Serum samples were used for control group analysis which are collected from age and sex matched healthy individuals (n=25).

v. Western Blot:

Western blotting was performed using a standard protocol. Cultured cells were harvested by the treatment of trypsin-EDTA (Life Technologies, Carlsbad, CA, USA) at different time points and were washed twice with PBS, then lysed in ice-cold RIPA buffer (Sigma-Aldrich, St. Louis, MO, USA) with a protease inhibitor (ThermoFisher Scientific, Waltham, MA, USA) and phosphatase inhibitor cocktail (Sigma-Aldrich, St. Louis, MO, USA). The total protein content of the extract was quantified using NanoDrop™ 2000 (ThermoFisher Scientific, Waltham, MA, USA). Cell lysates (approximately 20 μg of protein) were loaded by SDS-PAGE and transferred into a nitrocellulose membrane (0.45 mm pore size, ThermoFisher Scientific, Waltham, MA, USA). The membrane was blocked using 0.05 g/mL blotting-grade milk powder (Bio-Rad, Hercules, CA, USA) for two hours and then incubated with primary antibody for overnight incubation on an orbital shaker. After overnight incubation, the antigen-antibody complex was visualized with HRP-conjugated goat anti-rabbit or anti-mouse IgG (Cell Signaling, Beverly, MA, USA), then developed with an ECL detection system (Supersignal™ West Pico PLUS, ThermoFisher Scientific, Waltham, MA, USA) using the Bio-Rad ChemiDoc imaging system.

vi. Statistical Analysis:

Statistical analysis was performed using by GraphPad Prism software version 8 (GraphPad Software, Inc., La Jolla, CA, USA). All experiments were performed 3 independent times with fresh cultures of cells each time to obtain 3 replicates. The variables were investigated using visual (histograms, probability plots) and analytical methods (Kolmogorov-Smirnov/Shapiro-Wilk tests) to determine whether they were normally distributed. Kruskal-Wallis test was used to compare exosomal concentration size, expression of Glypican3, AR6 and CD81 in healthy, cirrhosis, and HCC groups. A value of P<0.017, calculated by Bonferroni correction, was considered statistically significant in this triple comparison. The Mann Whitney U test was used to evaluate differences between the two study subgroups. The statistical significance was shown as * p<0.05, ** p<0.01, *** p<0.001.

2. Results

i. Immuno-Magnetic Affinity Assay for Capturing Microvesicles and Exosomes:

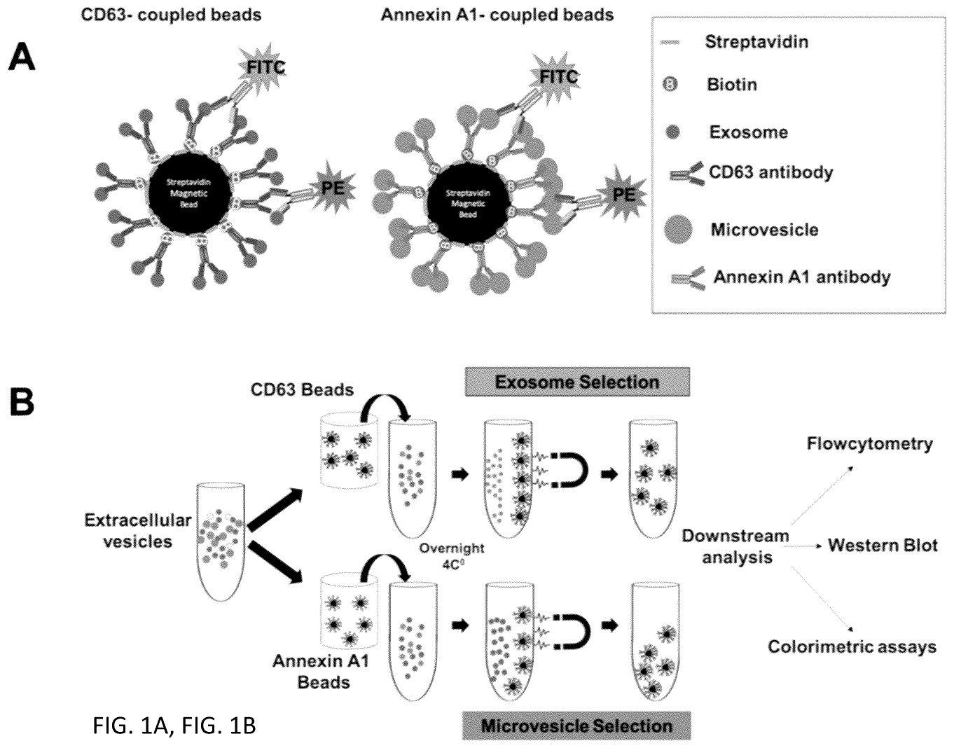

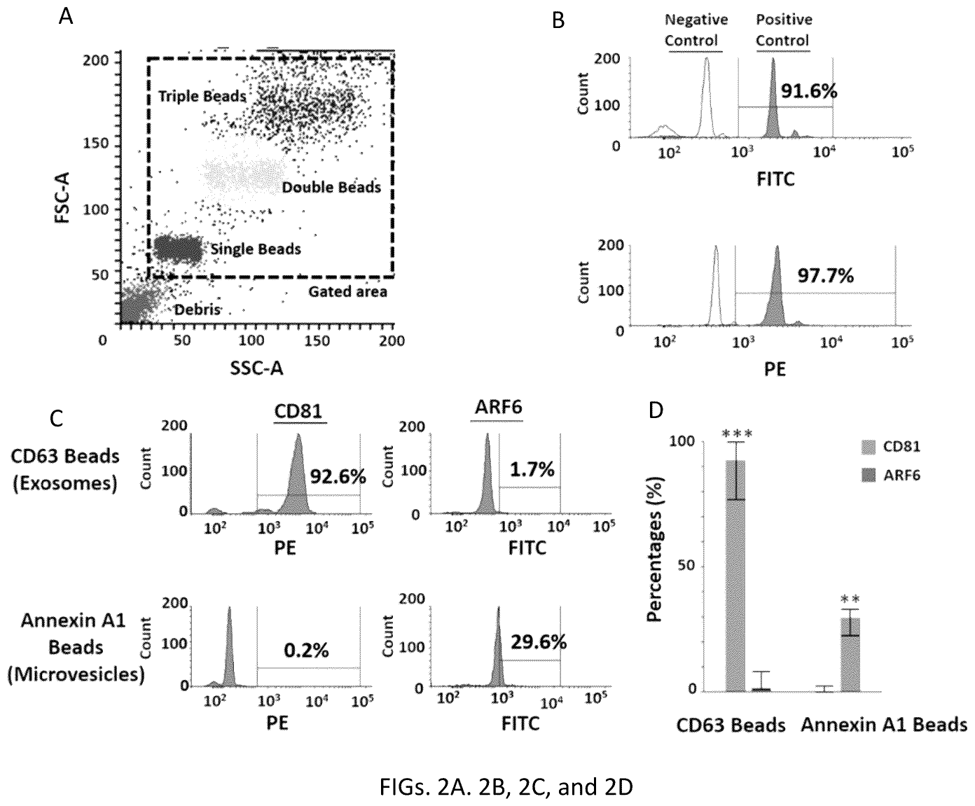

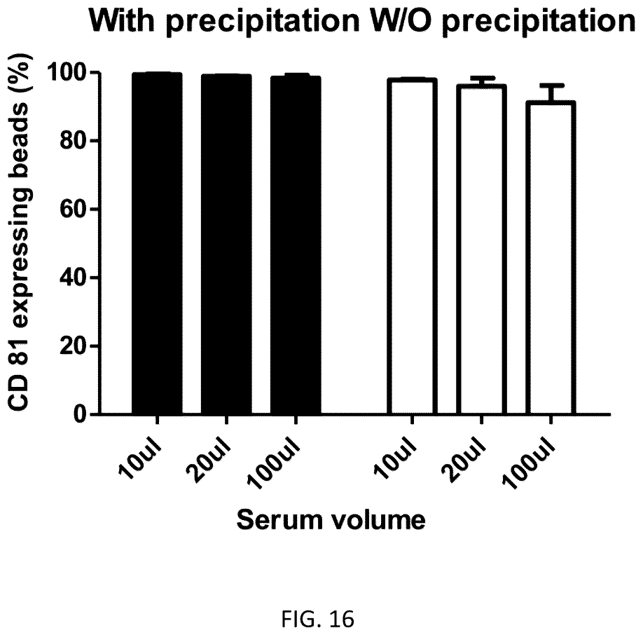

The clinical validation of EV-based biomarker discovery requires a highly reliable, reproducible, rapid turnaround, and cost-effective assay. Analysis of biomolecules in their native state is also essential to increase biomarker sensitivity. The method adopted here involved the use of a minimal amount of serum samples. Serum samples were diluted in PBS and centrifuged briefly to remove debris. Then serum samples were incubated with antibody-coated magnetic beads for overnight incubation at 40 C to capture exosomes/microvesicles. The next day, exosomes and microvesicles were selected by using a magnetic stand. Beads were washed two times using 500 microliters of PBS with 1% BSA. The captured exosomes or microvesicles were then stained with fluorescence-labeled CD81 or ARF6 antibodies for one hour. After this step, tubes were kept on a magnetic stand, and they were washed two times. The beads were resuspended in 500 microliters of PBS with 1% BSA and analyzed by a flow cytometer. For Western blot analysis, the beads containing exosome or microvesicles from step one were directly lysed in RIPA buffer, and Western blot analysis was performed using peroxidase-labeled antibodies. FIG. 1A shows a brief outline of the protocol for choosing microvesicles and exosomes. The immune-magnetic beads were prepared by mixing streptavidin-conjugated magnetic beads with biotinylated antibodies. After this step, beads were washed with filtered 1% BSA in PBS for blocking. The antibody-conjugated magnetic beads were incubated directly with the serum to capture exosomes or microvesicles overnight in a rotating shaker. The tubes were washed three times using a magnetic stand. In the final step, FITC-labeled CD81 antibodies or PE-labeled ARF6 antibodies were added (FIG. 1B). The number of microvesicles or exosomes is quantified by flow analysis. Initially, the minimum volume of serum samples needed for optimal detection of the exosomal marker was determined by flow cytometry. This assay was sensitive to detect exosomal cargoes using a minimum 10-μl of serum. Increasing the amount of serum volume did not show additional sensitivity to detection. An additional EV purification step before the immune-affinity flow isolation did not offer increased sensitivity (FIG. 16). FIG. 16 shows there was no significant difference between isolation/precipitation and without isolation/precipitation groups in terms of exosomal CD81 expression. Additionally, serum volumes were not correlated with exosomal CD81 levels. Forward Scatter (FSC)/Side Scatter (SSC) signals of the flow cytometer were used as a gating strategy to exclude aggregated beads. Fluorescence signals representing single, double and triple positive beads were analyzed (FIG. 2A). The quality of streptavidin-conjugated beads was assessed by incubating naked beads with biotinylated FITC or biotinylated PE fluorochrome. A stronger signal representing 91.6% positivity and 97.7% was observed when FITC-conjugated biotin and PE-conjugated biotin used (FIG. 2B). These data confirmed that the binding capacity of the streptavidin-coated magnetic beads used in the assay is comparable. The specificity of assay in detecting the exosomes and microvesicles in HCC cell culture was determined. As shown in FIG. 2C, the affinity isolation method can distinguish microvesicles (ARF6+/CD81−) from exosomes (ARF6−/CD81+). CD63 antibodies magnetic bead detects 92.6% of exosomes compared to 1.7% of microvesicles. Annexin A1 antibody-conjugated magnetic beads could catch 29.6% of microvesicles with a very negligible quantity of exosomes (0.2%). The affinity assay for exosome and microvesicle is consistent and reproducible since similar results were obtained by three repeated analyses (FIG. 2D). The method allowed for conventional flow cytometry detection of microvesicles and exosomes.

ii. Glypican-3 Enriched in Exosomes, not in Microvesicles:

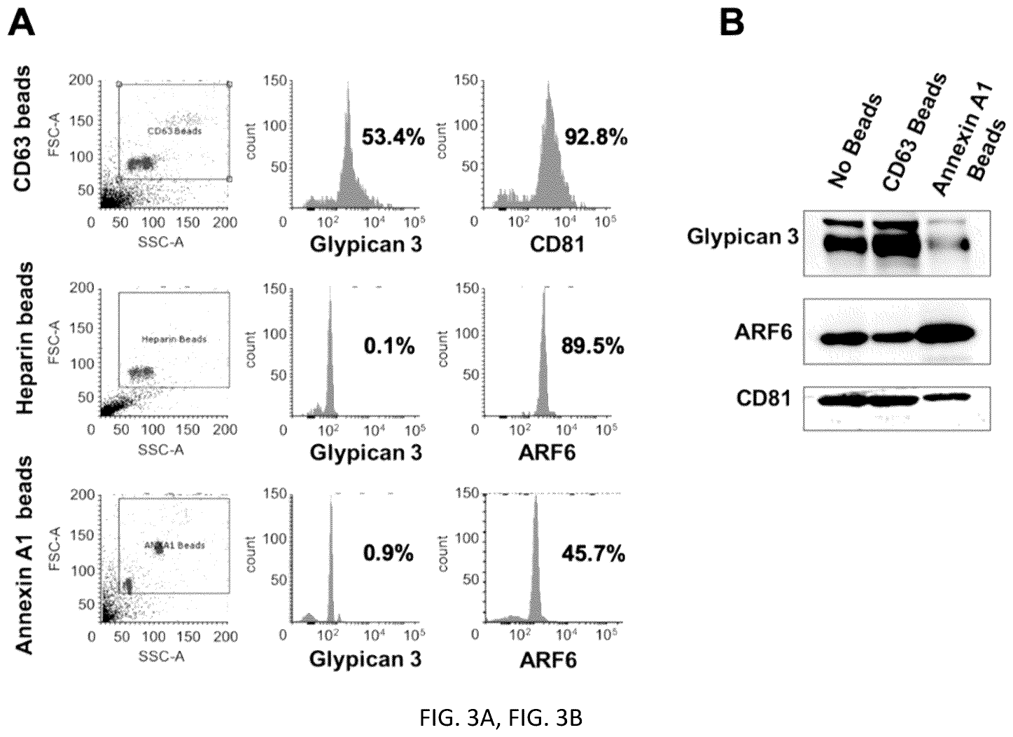

The GPC3 exosome is a serum biomarker for HCC detection in liver cirrhosis (25). Since tumor cells release both microvesicles and exosomes, GPC enrichment in microvesicles was tested. The immune affinity flow assay examined the presence of glypican-3 in microvesicles and exosomes in HCC cell culture supernatants. Exosomes were enriched by ExoQuick precipitation (Invitrogen). One hundred microliters of EV preparation were incubated with either Annexin A1 or CD63 coupled magnetic beads. The presence of GPC3 in exosomes and microvesicles was analyzed by flow cytometry. Nearly 53.4% of CD63 conjugated beads show positive for GPC3. The 92.8% of CD63 beads became positive for CD81 (FIG. 3A). In contrast, only 0.1% of heparin-coated beads showed positivity for GPC3, and 89.5% showed ARF6 positive (FIG. 3A). Only 0.9% of Annexin conjugated beads showed positive for glypican-3, and 45.7% showed ARF6 positive (FIG. 3A). These data indicate that GPC3 is preferentially enriched in exosomes compared to microvesicles. The enrichment of GPC3 in immuno-immobilized exosomes and microvesicles was examined by Western blot analysis. The immune-captured exosomes and microvesicles were solubilized, separated on the SDS page, and then examined by Western blot analysis using antibodies to GPC3, CD81, or ARF6 (FIG. 3B). A strong GPC3 signal was detected in the exosomes compared to microvesicles. ARF6 enriched preferentially in microvesicles and CD81 enriched in exosomes. These results show that the affinity purification approach accurately separates microvesicles from exosomes.

iii. Only Hepatocellular Carcinoma Cells Secrete GPC3 Positive Exosomes:

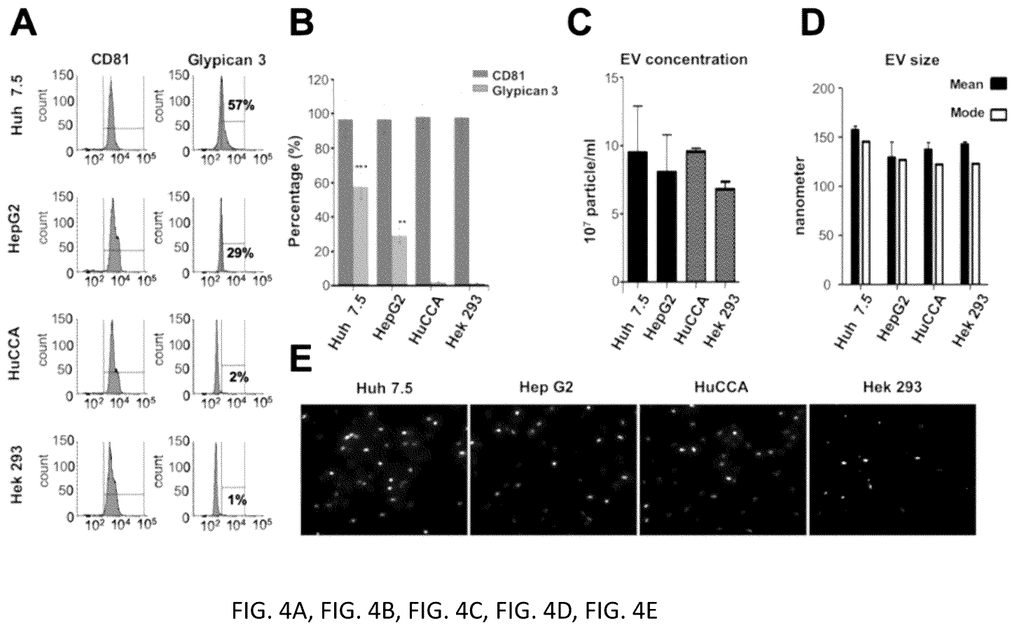

Extracellular vesicles were purified from culture supernatants of hepatocellular carcinoma cells (Huh-7.5, HepG20, cholangiocarcinoma (HuCCA) and HEK293 cells by precipitation using ExoQuick. Exosomal glypican-3 was compared using HCC and non-HCC tumor cell lines addressing the question of whether glypican3 exosomes could show specificity for HCC. The presence of exosomal glypican-3 between HCC and non-HCC cancer cells was quantified by flow analysis (FIG. 4A). A high percentage of GPC3 positive exosomes were detected in Huh-7.5 (57%) and HepG2 (29%) cells as compared to HuCCA (2%) and HEK293 (1%) cells. Exosomes released from hepatocellular carcinomas, cholangiocarcinoma, and human embryonic kidney cells were examined for the CD81 expression by flow analysis (FIG. 4B). The total EV production was measured to exclude the possibility that decreased GPC3 positivity is not due to the impaired production of exosomes or microvesicles in non-hepatic cells. The EVs isolated from cell culture were counted and verified in size by nanoparticle tracking analysis. These experiments show that the EV concentration and size among all cancer cells are comparable among all tumor cell lines (FIGS. 4C and D). Nanoparticle tracking analysis shows the size differences are not related to the production of exosomes or microvesicles (FIG. 4E). These data support the claim that glypican-3 exosomes are produced by HCC cell lines offering high specificity for HCC detection.

iv. Autophagy Induction Degrades GPC3, Whereas Autophagy Inhibition Accumulates its Expression in HCC Cells:

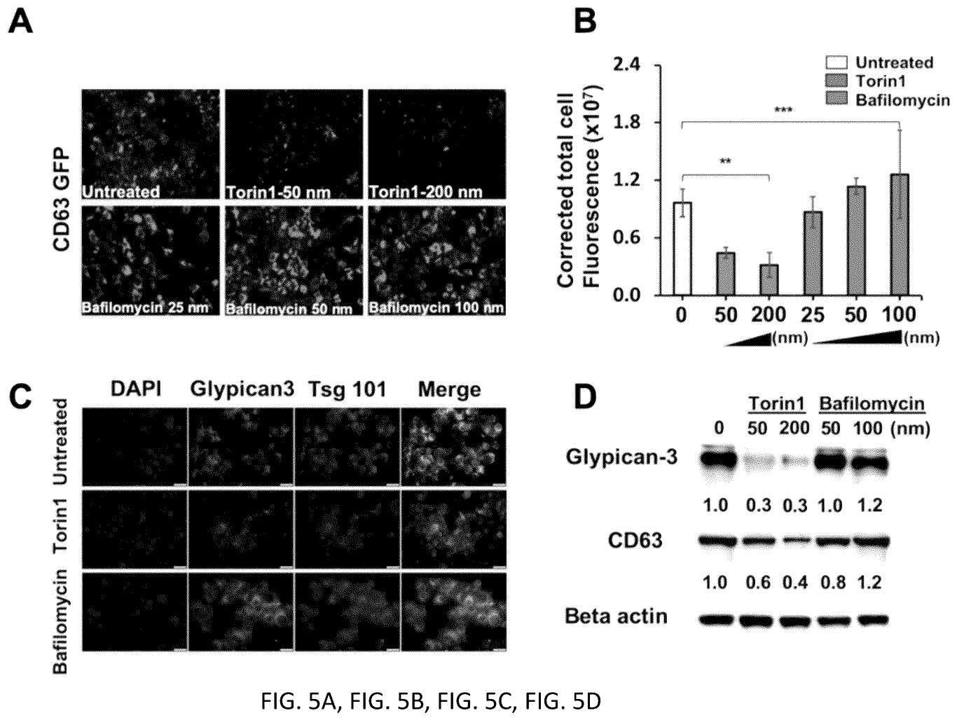

Exosomes are derived from the endosomal compartment. They are released by tumor cells where intraluminal vesicles fuse with the plasma membrane. Microvesicles are generated by the budding of vesicles from the plasma membranes. EV release and lysosomal degradation are two critical cellular events that are affected when autophagy is impaired in HCC (26). It has been demonstrated that autophagy controls the EV release using an HCV cell culture model. Using CD63-GFP stable cell lines, autophagy induction after hepatitis C virus (HCV) infection efficiently degrades MVBs and promotes EV release. To study the impact of autophagy on the GPC3 enrichment in HCC cells, the expression after treatment with autophagy inducer (Torin1) and inhibitor (Bafilomycin) was examined. Fluorescence microscopy assays demonstrate that autophagy induction by Torin1 degrades CD63-GFP chimera protein. Autophagy inhibition by Bafilomycin A1 induced its expression (FIG. 5A and FIG. 5B). Induction of autophagy in HCC cells by Torin1 degrades GPC3 and Tsg101. Autophagy inhibitor Bafilomycin A1 accumulates GPC3 expression in Huh-7.5 cells (FIG. 5C). These results were verified by Western blot analysis. For this purpose, Huh-7.5 cells were treated with two different concentrations of Torin and Bafilomycin A1 for 72 hours, and the expression level of GPC3 and CD63 levels were examined (FIG. 5D). All these data show that GPC3 is enriched in the endolysosomal compartment due to the impaired autophagy response in HCC.

v. Autophagy Induction Decreases GPC3 Positive Exosomes, Whereas Autophagy Inhibition Increases GPC3 Positive Exosomes:

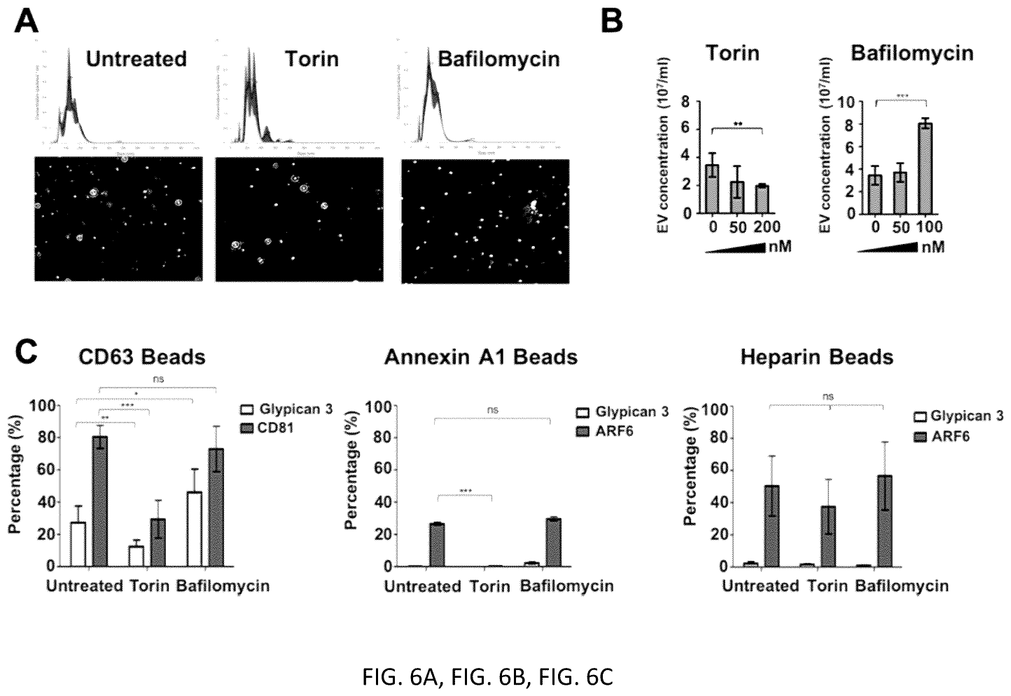

The bead-based assay was used to verify the production of microvesicles and exosomes by autophagy modulation. Huh-7.5 cells were treated with Torin 1 or Bafilomycin A1 for 72 hours. Exosomes were enriched from 1 ml culture supernatants by ExoQuick (Invitrogen). The concentration and size of the EVs preparation were visualized using a Nanoparticle tracking measurement (FIG. 6A and FIG. 6B). The impact of autophagy modulation on the production of exosomes and microvesicles was quantified by flow analysis. For this purpose, an equal amount of EV was incubated with either Annexin A1 or CD63 beads. The next day captured EVs were analyzed after staining with CD81-FITC or ARF-PE by flow cytometry. Consistent with our prediction, autophagy induction decreased total exosome production and glypican3 positive exosomes. Inhibition of autophagy increases the production of exosomes, and GPC3 positive exosome production increases in the HCC cell line. Decreased GPC3 positive exosomes were observed, and bafilomycin treatment increased exosome release and glypican-positive exosomes (FIG. 6C). In contrast, autophagy induction was found to promote ARF6 positive microvesicles. GPC3 positivity remained very low, indicating that GPC3 is not enriched in microvesicles captured by Annexin V1. Heparin bead-based microvesicle isolation method showed similar results. Autophagy induction and inhibition release ARF6 positive microvesicles, but the number of GPC3 positive microvesicles are very low. Taken together, the in vitro experiments demonstrated the specificity of magnetic bead-based affinity isolation protocol differentiating microvesicles from exosomes without purification by ultracentrifugation. The data from HCC cell culture show that glypican-3 exosome production in HCC cells relates to impaired autophagy.

vi. Glypican3 Enriched in Exosomes, not in the Microvesicles in the Cirrhotic Patients Who Developed HCC.

The contribution of GPC3 distribution in exosomes and microvesicles was verified using pathologically relevant human specimens. Serum samples of 25 healthy individuals were used, 25 with liver cirrhosis without HCC, and 25 with cirrhotic individuals with HCC. The immune affinity approach was utilized to detect GPC3 positive exosome and microvesicles by flow analysis. EVs were isolated from 10-microliters of serum samples using commercially available ExoQuick reagents. The concentration and size of EVs isolated from normal healthy individuals and cirrhosis without and with HCC were determined by nanoparticle tracking assay using Nanosight (FIG. 6A). The EV concentration increased in liver cirrhosis patients with HCC compared to the healthy individuals (FIG. 6B). There was no difference in EV size distribution in the different pathological stages of liver disease. The GPC3 positivity in exosomes and microvesicles was analyzed using serum samples of control and HCC. This affinity flow assay detected glypican-3 exosomes only in HCC, with positivity from 18% to 32%. In contrast, glypican-3 detection in microvesicles was minimal using Annexin A1 positive magnetic beads (FIG. 6C). A reasonably high percentage of CD81 positive exosomes were found in normal healthy, cirrhosis, and HCC samples (FIG. 6D). The rate of CD81 positivity in microvesicles is low in all groups. These data provide a strong rationale for CD63-affinity assay for detecting GPC3 in exosomes in HCC samples by flow cytometry. The immuno-magnetic separation is an easy and reliable approach for separating exosome/microvesicles. Immuno-magnetic affinity isolation can be used for HCC biomarkers screening.

3. Discussion

Extracellular vesicles are now new frontiers of biomedical research. The release of EVs is triggered during various pathological conditions, including cancer. There has been a lot of interest in identifying EV-enriched molecules as biomarkers in cancer prognosis and diagnosis. EVs circulating in the blood are quite heterogeneous in their origins and molecular composition, making it difficult for biomarker selection. This study addressed whether GPC3 positive exosome is a biomarker for HCC diagnosis. The development of a convenient method for high-throughput EV analysis is needed for biomarker validation in clinical samples. Currently, various methods have been described in the literature for EV analysis. Ultracentrifugation is the most commonly used method to purify exosomes. This method cannot be adopted for the large-scale handling of serum samples in clinical laboratories. The direct precipitation of exosomes in clinical samples using a polymer-based precipitation reagent offers an alternative. This method precipitates many serum proteins and non-extracellular vesicles. Exosomes isolated by this method are not pure, and serum protein contaminants may affect downstream analysis. Development of a highly sensitive, reliable, cost effective protocol to detect biomolecules in EV in native form with a rapid turnaround time is needed. To overcome these problems, a sensitive and reproducible method for the detection of exosomes and microvesicles was developed. This method is convenient as compared to standard methods used earlier. Immune affinity isolation protocol was shown to accurately differentiate exosomes from microvesicles. This method can be tested using a small amount of clinical specimens, as little as 10-microliters of serum. The conventional flow assay can be adapted to any clinical laboratory. Use of magnetic beads provides an advantage in performing extensive washing to isolate a very pure population of exosomes and microvesicles, which offers a solution to complex EV-based biomarker discovery.

The affinity flow assay shows that glypican-3 molecules are enriched in exosomes, not in the microvesicles. Using cell-based assay, it was shown that autophagy induction decreased exosome release whereas impaired autophagy accumulated GPC3 in exosomes. These results show why GPC3 exosome numbers are released among cirrhotic patients who developed HCC. Using serum samples of cirrhotic patients with HCC, it was shown that GPC3 are enriched in exosomes, not in the microvesicles. Impaired autophagy has been implicated in HCC development in humans and mouse model. Development of serum exosome-based biomarkers that measures the impaired autophagy response in the liver could be a powerful marker for HCC detection and treatment.

Impaired autophagy increased exosome release, and GPC3 is enriched in HCC exosomes. Surprisingly, GPC3 is not detected in microvesicles, indicating that glypican-3 plays a major role in multivesicular body biogenesis and exosome release. Glypicans are heparan sulfate proteoglycans (HSPGs) attached to the extracellular surface of the plasma membrane through a glycosyl-phosphatidylinositol (GPI) anchor. There are six members of glypicans (GPC1-6). Glypicans preferentially localize to cholesterol-rich lipid rafts and caveolae in the membrane, leading to enhanced cell signaling, active endocytosis and recycling. Glypican-3 involved in the endocytois and degradation of GPC3-Hh complex. The low-density-lipoprotein-related receptor-1 (LRP-1) mediates the Hh-induced endocytosis of the GPC-Hh complex. This data is in agreement with this report showing that autophagy induction promotes fusion of endosome-autophagosome-lysosomes degrade GPC-3. Impaired autophagy prevents GPC3 degradation leading to its accumulation in multivesicular bodies and release through exosomes in HCC. The immune-magnetic bead-based detection of HCC exosomes is a valuable and sensitive method that can be extended to use in the early detection of cancer and treatment.

### B. Example 2: Experimental Validation of Novel Glypican 3 Exosomes for the Detection of Hepatocellular Carcinoma in Liver Cirrhosis

Described herein are HCC-derived exosomes that were enriched with GPC3 and levels of exosome-derived GPC3 (eGPC3) in serum provide superior diagnostic performance compared to AFP. This study shows that HCC-specific exosomes detection is an accurate serum biomarker for HCC diagnosis in patients with cirrhosis.

1. Methods

i. Immunohistochemistry (IHC):

Five-micron tissue sections were prepared from paraffin embedded HCC tissues and control non-HCC liver. Tissue sections were deparaffinized for 15 minutes at 50-600 C followed by treatment with xylene twice for 5 minutes. The tissue sections were rehydrated by sequential treatment with 100%, 95%, and 80% alcohol. Peroxidase quenching was carried out by incubation with 3% hydrogen peroxide and 100% methanol for 5 minutes. The slides were placed in a plastic Coplin jar with Reveal Decloaker RTU (RV 1000 Biocare Medical) for 25 minutes at 95° C. in a steamer for heated antigen retrieval. Following this step, the slides were allowed to cool at room temperature for 20 minutes. The tissue sections were rinsed in deionized, distilled water and marked using a PAP pen. The slides were incubated with a blocking sniper (Biocare Medical) for 10 minutes and incubated with a primary antibody for 1 hour at room temperature. The primary antibodies used were SQSTM1/p62 mouse monoclonal antibody in 1:200 dilution (88588S, Cell signaling) and pre-diluted antibody to glypican-3 (PM396 AA, Biocare Medical). After the primary antibody incubation, slides were washed 3 times in Tris Buffer Saline (TBS) (pH 8.0), incubated with a MACH 4 mouse probe (UP534, Biocare Medical,) for 20 minutes and MACH 4 HRP Polymer (Biocare Medical, MRH534) for 30 minutes each, then washed 3 times using TBS. Finally, Diaminobenzidine (DAB) chromogen reagent (BDB2004L, BioCare Medical) was used to develop the staining for 1-5 minutes. The slides were then counterstained with Hematoxyln (Biocare Medical, CATHE-M) for 30 seconds and Tacha's bluing Solution (Biocare Medical, HTBLU-M) for 30 seconds, mounted, and observed by light microscopy. H&E-stained sections of all specimens, including cancer and non-cancer cases, were examined, and scored independently by two pathologists who are experienced in hepatobiliary pathology following the IHC evaluation. Scores were assigned to the intensity and percentage of positive staining. Scoring as follows: 0 means negative staining, 1 (weak), 2 (medium) and 3, (strong). Multiplying the intensity of score and proportion of immunopositive cells (0-100%), a semi quantitative staining score ranged from 0 to 300 was established for statistical analysis.

ii. Cell Lines and Chemicals:

All cell lines were purchased commercially. Primary human hepatocytes (PHHs) were cultured with hepatocyte culture media supplemented with 10% human serum (Invitrogen, Brown Deer, WI). HCC cell lines Hep G2 cells, Huh-7, Huh-7PX, and Huh-7AF, HLE and HLF Huh-7.5 were grown in Dulbecco's Modified Eagle's Medium supplemented with 10% fetal bovine serum, nonessential amino acids, sodium pyruvate, and penicillin, streptomycin, and amphotericin B (Gibco, Carlsbad, CA). Torin1 (475991) was purchased from Sigma-Aldrich.

iii. Immunocytochemistry and Western Blotting:

PHHs or HCC cells were immobilized on glass slides and stained for p62 and glypican 3 by immunostaining. Exosomes and adherent cells were separated isolated from the cultures and lysates (20 μg of protein) prepared and examined for expression of p62, glypican 1, glypican 3 and exosome markers, CD9, TSG101 by SDS-PAGE electrophoresis and Western blotting. Cells were lysed in ice-cold lysis buffer (50 mM Tris HCl pH 8.0, 140 mM NaCl, 1.5 mM MgCl2, 0.5% NP-40 with complete protease inhibitor from Invitrogen) for 10 minutes in ice (about 1×106 cells/200 μL). Cells pelleted by low-speed centrifugation. The detergent compatible (DC) protein assay determined protein concentration. Samples were boiled for 10 minutes at 80° C. in the presence of 1× sodium dodecyl sulfate-polyacrylamide gel electrophoresis (SDS-PAGE)-loading buffer (250 mM Tris-HCl pH 6.8, 40% glycerol, 8% SDS, 0.57M β-mercaptoethanol, 0.12% bromophenol blue). Approximately 20 μg of protein was loaded onto 12% SDS-PAGE and transferred into a nitrocellulose membrane (LC2001 Thermo Scientific). The membrane was blocked using a solution containing 5% of blotting-grade milk power (Bio-Rad, Hercules, CA, USA) for 2 hours then incubated with a primary antibody. Antibodies to p62 (sc-28359), glypican 3 (sc-65443), glypican 1 (sc-365000), TSG101 (sc-7964), CD9 (sc-13118) and glyceraldehyde-3-phosphate dehydrogenase (GAPDH) (sc-365062) were purchased from Santa Cruz Biotechnology. After overnight incubation at 40° C. on a rocker, the antigen-antibody complex was visualized with horseradish peroxidase (HRP)-conjugated goat anti-rabbit or anti-mouse Immunoglobulin G (IgG) and the ECL detection system (RPN2232 Amersham ECL, GE Healthcare Biosciences, Pittsburgh, PA, USA).

iv. DQ-Red BSA Staining:

DQ™ Red BSA (D12051, Thermo Scientific) is a fluorogenic substrate that is endocytosed in cells and traffics through early endosomes to late endosomes that then fuse with acidic hydrolase containing lysosomes. This leads to the formation of endo-lysosomes that degrade DQ-Red BSA, de-quenching the fluorescence of the dye attached to this cargo, therefore, produces bright red spots in endo-lysosomes. Equal number of cells was seeded in 6-well plate. Then, untreated and Torin1-treated live cells were incubated with DQ-Red BSA for one hour. After washing cells with PBS, cells were fixed with 4% paraformaldehyde and counterstained with Hoechst 33342 (H3570, Thermo Scientific). Cells were examined using fluorescent microscopy.

v. Exosome Isolation from Cell Culture Media:

Fetal Bovine Serum-free conditioned cell culture media were collected and centrifuged at 2000×g for 30 minutes to remove cells and debris. Exosomes were isolated using the Total Exosome Isolation Reagent for cell culture supernatant (4478359, Invitrogen™, Thermo Fisher Scientific®, Waltham, MA, USA). According to the manufacturer's instructions, 0.5 volume of reagent for 1 volume of cell culture media were vortexed. The samples were incubated overnight at 40 C. The next day samples were centrifuged at 10.000×g for 1 hour at 40° C. then supernatant was discarded. The exosome pellet was retrieved after removal of the supernatant and resuspended in 100 μl 1× phosphate buffered saline (PBS) for downstream analyses.

vi. Exosome Isolation from Serum:

Exosomes were isolated using the exosome Isolation Reagent (4478360, Invitrogen™, Thermo Fisher Scientific®, Waltham, MA, USA) according to the manufacturer's instructions. Briefly, serum samples were centrifuged at 2000×g for 30 minutes to remove cell and debris. The serum samples then were filtered through 0.22 μm syringe filter (Millipore). 0.2 volumes of total exosome isolation reagent were added to serum (40 μl of isolation reagent to 200 μl of serum) and mixed the serum-reagent mixture by vortexing. The samples were incubated for 30 minutes at room temperature. After incubation, the samples were centrifuged at 10,000×g for 10 min at 4° C. The exosome pellet was retrieved after removal of the supernatant and resuspended in 100 μl PBS for downstream analyses.

vii. Nanoparticle Tracking Analysis:

Nanoparticle tracking analysis (NTA) is a powerful technique that measures both laser light scattering and the Brownian motion of the exosomes in the liquid phase. NanoSight NS300 instruments (Malvern, UK) was used to visualize by the light scatter by exosomes after laser illumination, and their Brownian motion. Exosomes are secreted after the fusion of MVBs with the plasma membrane, whereas microvesicles are thought to bud directly from the plasma membrane. The majority of EVs occur within the sub-micron range (30-1000 nm) where exosomes are believed to be most abundantly present in the lowest size range (30-150 nm). The NTA software enables the sizing of single particles by tracking their mean squared displacement and thereby calculating their theoretical hydrodynamic diameter using the Stokes-Einstein equation. Based on knowing the sample volume, NTA also allows for an accurate estimation of particle concentration. Exosome samples were diluted in 1:1000 and the suspensions were passed through a flow chamber and are illuminated using a laser source. Video recorded for each sample was analyzed with NTA software version 2.3 to determine the concentration and size of the measured particles with the corresponding standard error. NanoSight system was calibrated with polystyrene latex microbeads of 50,100 and 200 nm (Thermo Scientific) before analysis.

viii. LC-MS Analysis:

A high sensitivity liquid chromatography-mass spectrometry (LC-MS/MS) analysis of extracellular vesicles derived from human hepatocellular carcinoma cells (Huh-7.5) was performed to determine the list of proteins enriched in HCC exosomes using a high sensitivity LC-MS/MS. A protein identification software Mascot search engine (Version 2.2.07) was used for mass spec data analysis.

ix. Cryogenic Transmission Electron Microscopy (CryoTEM) and Immunogold Labeling:

The exosome samples were blotted on a lacey carbon-coated copper grid (200-mesh, electron microscopy sciences) using an automated system, Vitrobot (FEI, Hillsboro, OR), operated at 100% humidity for 2 seconds. For immunogold labeling, exosomes were first blocked with 0.5% BSA and then successively incubated with mouse anti-glypican antibodies followed by rabbit anti-mouse (Dako, Glostrup, Denmark). The next, samples were incubated by 10-nm gold particles (Cytodiagnostics) for 2 h at room temperature. Samples were then contrasted and embedded in a mixture of 0.4% uranyl acetate and 1.8% methylcellulose. The grids were washed with PBS followed by double distilled water and stained with 0.4% uranyl acetate/1.8% methylcellulose and then dried. Exosomes were observed using a Tecnai™ G2 Spirit BioTWIN transmission electron microscope (TEI) and images were taken with an AMT CCD camera (Advanced Microscopy Techniques, Woburn, MA, USA) at 150 kV.

x. Bead-Based Exosome Immunoaffinity Capture and Flow Analysis:

A 1 μL volume of streptavidin latex beads (Invitrogen) was washed with PBS and incubated with biotinylated anti-CD9 for one hour. The beads were washed three times with PBS and blocked with 100 mM glycine and 2% BSA for one hour at room temperature. The beads were suspended in PBS and added to exosomes to a final concentration of 108/mL for overnight incubation at 4° C. in a shaker. The next day beads were washed three times using 2% BSA in PBS. For the detection of exosome proteins, beads were incubated with Laemmli buffer, boiled, and subjected to Western blot analysis. For flow analysis, affinity-purified exosomes were washed once in 2% BSA in PBS and centrifuged at 14,800×g for one minute and blocked with 10% BSA while rotating at room temperature. Purified exosomes were then washed with 2% BSA in PBS, centrifuged at 14,800×g for one minute, and incubated with primary antibody to either glypican 1 (sc-36500, Santa Cruz) or glypican 3 (MA5-16368, Thermo Scientific) for one hour while rotating at 4° C. Following centrifugation at 14,800×g for one minute, the beads were washed with 2% BSA in PBS and stained with Alexa fluor 488-conjugated antibody (Invitrogen) for one-hour with rotation at 4° C. Beads were washed three times with 2% BSA in PBS and then analyzed by flow cytometry. Secondary antibody incubation alone with exosomes was used as control and to establish positive staining gates in the acquisition and analysis software.

xi. Serum Cohort Study:

Peripheral blood specimens were obtained through a prospective study approved by the Ochsner Health Institutional Review Board (protocol 2016.131.B) and included 93 patients. This study was also conducted in accordance with the Declaration of Helsinki. HCC was diagnosed by biopsy or met the Liver Imaging Reporting and Data System (LI-RADS) class LR-4 or LR-5. All patients in the cohort were reviewed by a multidisciplinary tumor board and selected for bridge to transplant locoregional therapy with doxorubin-eluting bead transarterial chemoembolization (DEB-TACE). Blood was obtained immediately prior to procedure after obtaining informed consent for participation in the study. A second blood specimen was collected at routine CT/MRI follow-up visit 30-days following the DEB-TACE procedure. Patients with cirrhosis undergoing routine HCC surveillance were consented for the study and blood collected at 6-month visit following a normal ultrasound visit and normal serum AFP. Peripheral blood was collected, processed to obtain serum, and stored at ultra-low temperature within 2 hours of collection. Clinical data for cirrhosis etiology, tumor radiographic burden, as well as baseline and follow-up complete metabolic panel, complete blood count, and serum AFP values were extracted from the electronic medical record. Serum samples from individuals with non-HCC malignancy (n=10) were obtained from Louisiana Consortium Research Center with IRB approval (protocol 16-911367E). Serum samples from healthy controls (n=25) were obtained from Clinical Pathology of Tulane Health Sciences Center.

xii. Treatment Response to DEB-TACE Locoregional Therapy:

An interventional radiologist blinded to the purpose of the study following routine follow-up imaging independently assessed response to treatment. Treatment response was evaluated using the Modified Response Evaluation Criteria for Solid Tumors (mRECIST) for HCC.53 An objective response to treatment was defined by mRECIST of complete or partial response.

xiii. Exosome Immunocapture and Detection of Multiple Exosome Protein by Enzyme-Linked Immunosorbent Assay (ELISA):

A custom ELISA for exosome capture and multiplex analysis was developed for analyzing low volume serum samples. In brief, 96-well polystyrene ELISA plates were coated with a 1:1000 diluted CD63 polyclonal rabbit antibody (EXOAB-CD63A-1, System Biosciences) in bicarbonate/carbonate coating buffer (100 mM) overnight at 4° C. The plates were then washed with PBS three times and blocked with 100 μl of 1% BSA-PBS 1 hour at room temperature. Following three washes in PBS, cell culture- or serum-isolated exosomes were added in a final volume of 50 μl and incubated overnight at 37° C. After three washes in PBS, replicate samples were incubated with either anti-CD9 mouse monoclonal antibody, mouse monoclonal antibody to HSC70, or mouse monoclonal antibody to GPC3 each diluted to a concentration of 1 μg/mL and incubated for 1 hour at 37° C. After three washes with PBS, the plate was incubated with 100 μl of HRP-conjugated anti-mouse antibody at a dilution of 1:1000 in 1% BSA in PBS for one hour at room temperature. Following three washes with PBS, the reaction was developed with 100 μl of TMB substrate (7004, Cell Signaling) for 30 minutes. The reaction was halted with 50 μl of stop solution (H2SO4) and the optical density (OD) recorded at 450 nm within 30 minutes.

xiv. Statistical Analyses:

All statistical analyses were performed using Graphpad Prism software version 5.0 for Windows (GraphPad Company, San Diego, CA, USA). ImageJ software version 1.52p (NIH, Bethesda, MD, USA) was used to process the images derived from Western blot and immunostaining. The variables were investigated using visual (histograms, probability plots) and analytical methods (D'Agostino & Pearson omnibus normality test and Shapiro-Wilk test) to test for a normal data distribution. Ordinal variables and continuous variables failing tests for normal distribution were compared by the Mann-Whitney U test. Student's t-test was used to evaluate differences between the two groups when the variables were normally distributed. Wilcoxon analysis was used for comparing pretreatment and post-treatment glypican 3, CD9, and AFP levels in the serum cohort studies. Serum glypican 3 and p62 levels were used in Receiver operating characteristics (ROC) analysis to discriminate the non-cirrhotic controls, cirrhotic controls, confirmed HCC, and non-HCC malignancy groups. The coefficient of variation values in ELISA results were used to generate the ROC curve for locoregional treatment response prognosis in patients with HCC. Sensitivity, specificity, positive predictive, and negative predictive values were calculated for optimal cut-offs obtained by ROC analysis. All measurements were performed in triplicate (n=3). An overall 5% type-I error was used to infer statistical significance. P values were represented as ns P>0.05, *P<0.05, **P<0.01, ***P<0.001 in the figures.

2. Results

i. Elevated GPC3 and p62 Expression in Autophagy-Deficient HCC Tissue:

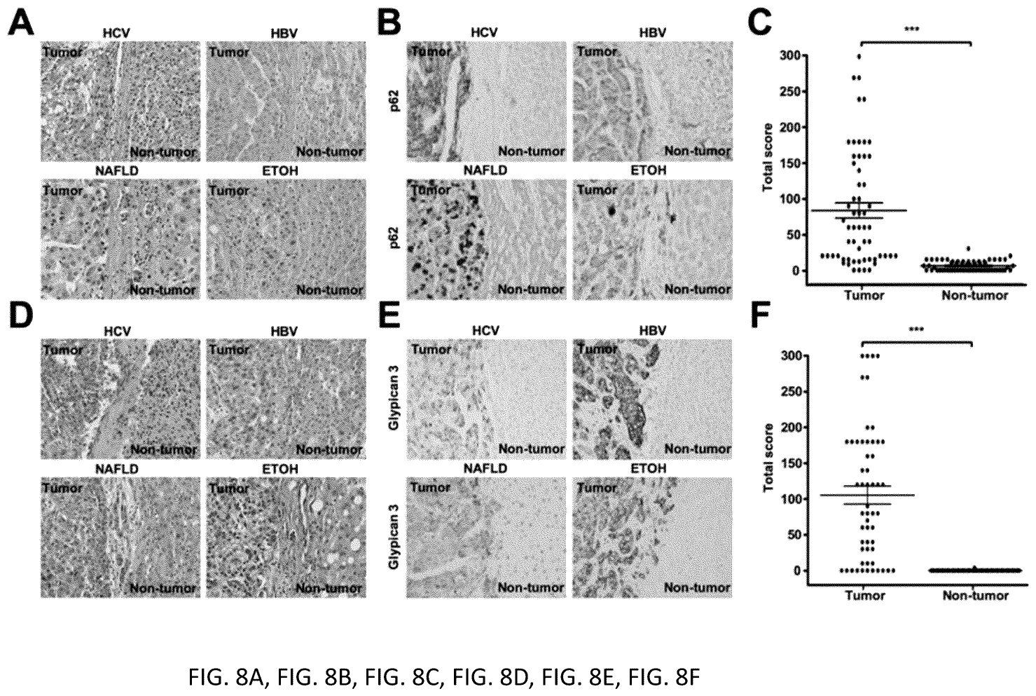

Histologic sections of previously confirmed and verified by pathologist, cases of HCC with adjacent cirrhotic liver were selected for the IHC (FIG. 8A and FIG. 8D, H&E, ×200). Impaired autophagy was confirmed, evidenced by increased expression of the autophagy flux protein p62. The expression of p62 was examined by IHC in 58 HCC specimens containing a reference margin to the cirrhotic liver. In total 89.6% of specimens evaluated, confirming impaired autophagic flux in HCC (FIG. 8B). Semiquantitative IHC scoring analysis revealed nearly 10-fold increased p62 expression in the HCC lesion compared to adjacent cirrhotic tissue (FIG. 8C). Immunostaining for GPC3 confirmed expression was restricted to the HCC lesion, with 80% of the specimens staining positive for GPC3 relative to negative stained cirrhotic liver background (FIG. 8E and FIG. 8F). There was not significant difference among the etiologies of cirrhosis in terms of p62 and GPC3 expressions (Table 1).

TABLE 1
Summary of p62 and glypican 3 expression in HCC and adjacent cirrhotic tissues

| | | P62 expression | | | | Glypican 3 expression* | | | |
|---|---|---|---|---|---|---|---|---|---|
| Etiology | Number | Tumor | | Non-tumor | | Tumor | | Non-tumor | |
| ETOH | 10 | 10/10 | (100%) | 5/10 | (50%) | 8/9 | (88.8%) | 0/9 | (0%) |
| HBV | 14 | 12/14 | (85.7%) | 6/14 | (42.8%) | 9/13 | (69.2%) | 0/13 | (0%) |
| HCV | 18 | 14/18 | (77.7%) | 8/18 | (44.4%) | 17/18 | (94.4%) | 0/18 | (0%) |
| NAFLD | 16 | 16/16 | (100%) | 10/16 | (62.5%) | 10/14 | (71.4%) | 1/14 | (7.1%) |
| Total | 58 | 52/58 | (89.6%) | 29/58 | (50%) | 44/54 | (81.4%) | 1/54 | (1.8%) |

ETOH: Ethanol,
HBV: Hepatitis B Virus,
HCV: Hepatitis C Virus,
NAFLD: Non-Alcoholic Fatty Liver Disease
*Immunostaining of glypican 4 was not performed in three patients due to the lack of enough tissue.

ii. Impaired Autophagic Flux with Increased p62 and GPC3 Expression in HCC Cell Lines:

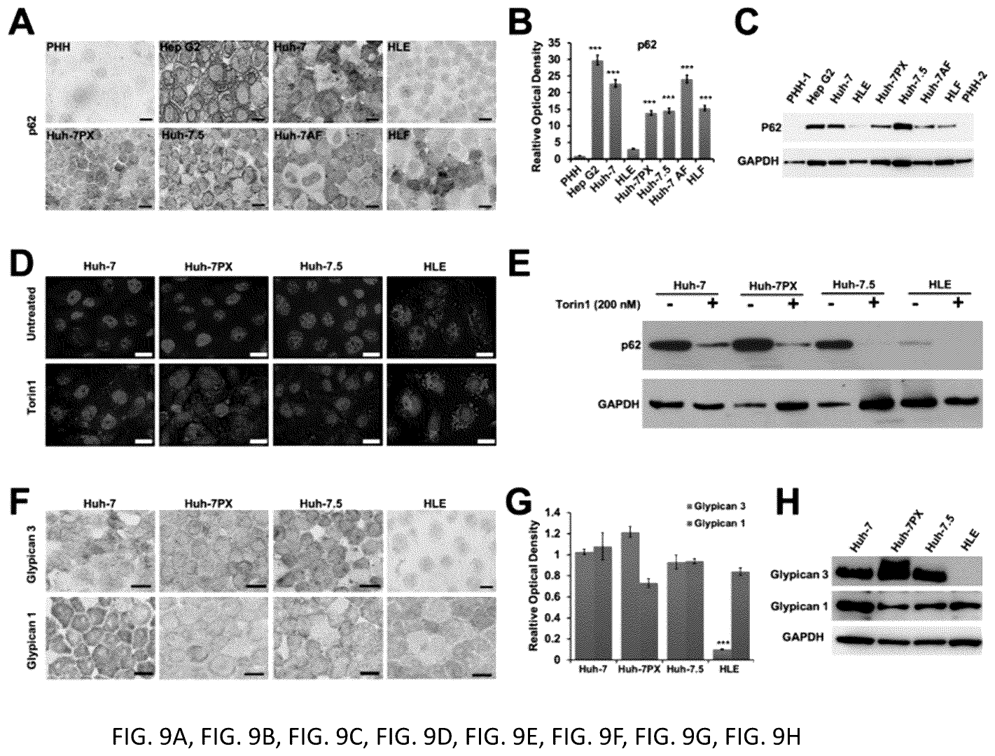

Next, it was confirmed whether HCC cell lines and non-transformed primary hepatocytes could recapitulate impaired autophagy and increased GPC3 expression specific to HCC. A panel of 6 HCC cell lines was examined for p62 expression levels compared to primary human hepatocytes (PHH) using immunocytochemistry (FIG. 9A). Productive autophagy was evident in PHH control cells as well as the HCC line HLE. However, all other HCC lines had prominent cytoplasmic staining for p62, evidenced by significantly higher staining OD (FIG. 9B), indicating defective autophagic flux. Elevated p62 protein expression was also confirmed by Western blot (FIG. 9C). DQ-Red BSA endocytosis assay with Torin1 stimulation was used to confirm productive autophagy between the autophagy-deficient HCC lines (Huh-7, Huh-7PX, and Huh-7.5) compared to autophagy-sufficient HCC cell line HLE. Autophagy-sufficient HLE cells demonstrated bright DQ-BSA fluorescence signal that increased after treatment with an autophagy inducer Torin1 (FIG. 9D). The autophagy-deficient HCC lines were negative for DQ-BSA fluorescence that could only be induced following Torin1 treatment. Torin1 decreased p62 levels in the autophagy-deficient HCC lines, confirming impaired autophagy (FIG. 9E). It was then verified that deficient autophagy is linked with increased GPC3 expression. HCC-specific GPC3 expression was examined by immunostaining along with expression of glypican 1 (GPC1), which is malignancy-associated but not HCC specific (FIG. 9F). Expression of GPC3 was restricted to the autophagy-deficient HCC lines while GPC1 was present in both the autophagy-sufficient and -deficient HCC lines Immunostaining OD for GPC3 was significantly elevated with reference to the HLE line (FIG. 9G). Immunostaining results for GPC1 and GPC3 between autophagy-sufficient and -deficient HCC lines were confirmed by Western blot (FIG. 9H), confirming a mechanistic link between impaired autophagy and GPC3 expression in HCC.

iii. Autophagy-Deficient HCC Lines Secrete GPC3-Enriched Exosomes:

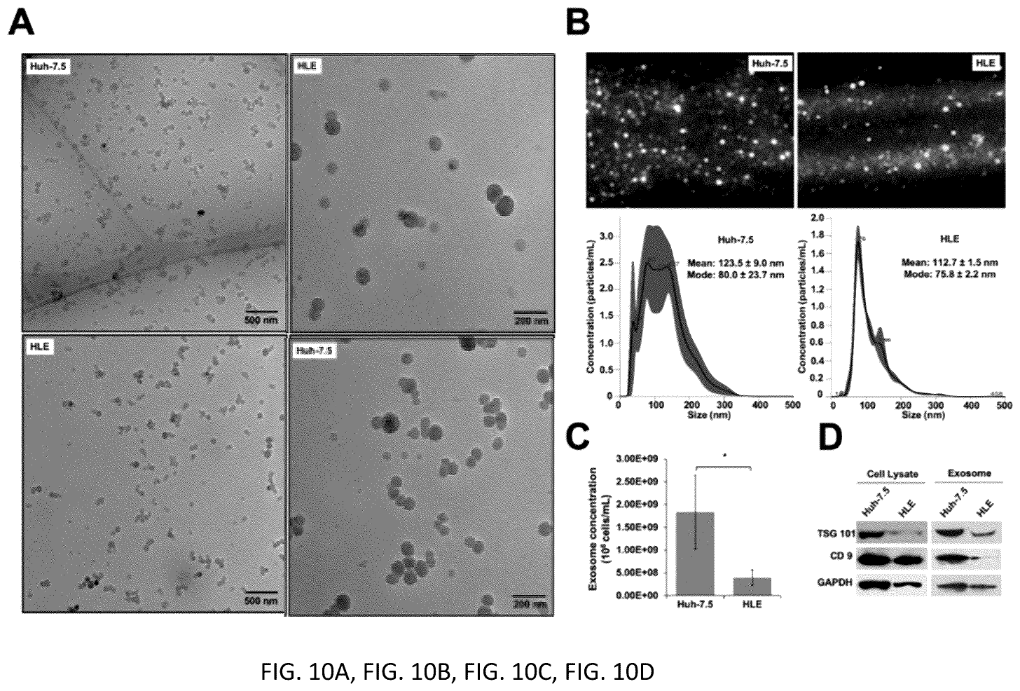

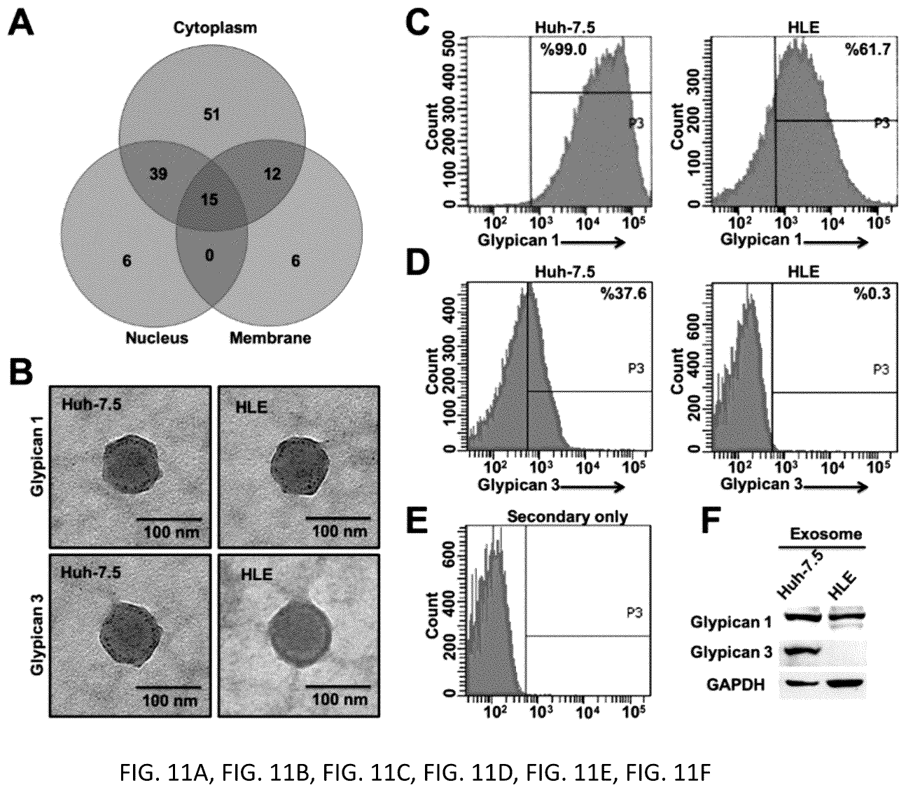

Exosomes isolated from the autophagy-deficient and -competent HCC lines were analyzed to confirm a relationship between autophagy status and exosome release. Exosomes were isolated from culture supernatants of HLE (autophagy-competent) and Huh-7.5 (autophagy-deficient) HCC lines and characterized by cryoTEM. Vesicular structures with a bilayer lipid membrane were detected in the isolates from both HCC lines. The bilayer lipid membrane morphology and vesicle diameter (30-150 nm) were consistent with cell-derived exosomes (FIG. 10A). NTA revealed the light scattering properties and Brownian motion of exosome from the HLE and Huh-7.5 cells in the liquid phase differed, although the exosome size between the cell lines were similar (FIG. 10B). Autophagy-deficient Huh-7.5 cells also secreted exosomes at a higher concentration per cell (P=0.03) compared to autophagy-competent HLE cells (FIG. 10C). Exosomes isolated both lines were characterized by Western blot for expression of the tetraspanin CD9, enriched in the exosome membrane, and TSG101, a protein involved in recruitment an internalization of cargo into MVBs (FIG. 10D). These analyses show that the exosome concentration is low in a cell line with active autophagy as compared to a cell line with defective autophagy. An LC-MS analysis was performed using exosomes isolated from Huh-7.5 cells. Content of the exosomes isolated from the Huh-7.5 autophagy-deficient line were further characterized using LC-MS. Bioinformatic analyses revealed 129 proteins including GPC3. The majority of the proteins are exclusively cytoplasmic in origin (51 proteins) or shared between the cytoplasmic and nuclear or membrane compartments (39 proteins cytoplasmic and nuclear, 12 proteins cytoplasmic and membrane-bound, and 15 proteins present in all compartments). An additional 6 proteins are exclusively nuclear and 6 others exclusively membrane-bound (FIG. 11A). Immunogold labeling was used to confirm the location of GPC1 and GPC3 in TEM preparations of exosomes isolated from autophagy-deficient Huh-7.5 and autophagy-competent HLE cells (FIG. 11B). In agreement with Western blot analysis from whole cell extracts, GPC1 expression in exosomes isolated from both Huh-7.5 and HLE, independent of autophagy status. GPC3 immunolabeling was specific to the autophagy-deficient Huh-7.5 cells. Exosome flow cytometry confirmed GPC1 expression in both preparations (FIG. 11C) with GPC3 expression restricted to exosomes isolated from autophagy-deficient Huh-7.5 (FIG. 11D). GPC3 staining of the exosomes were confirmed by complete loss of fluorescent signal when the primary antibody only was omitted (FIG. 11E). These results were also confirmed by Western blot analysis on the same culture-isolated exosome preparations (FIG. 11F). GPC3 expression restricted to exosomes derived from autophagy-deficient Huh-7.5 was confirmed by all methods evaluated.

iv. Impaired Autophagy Response in HCC Increases Exosome Release:

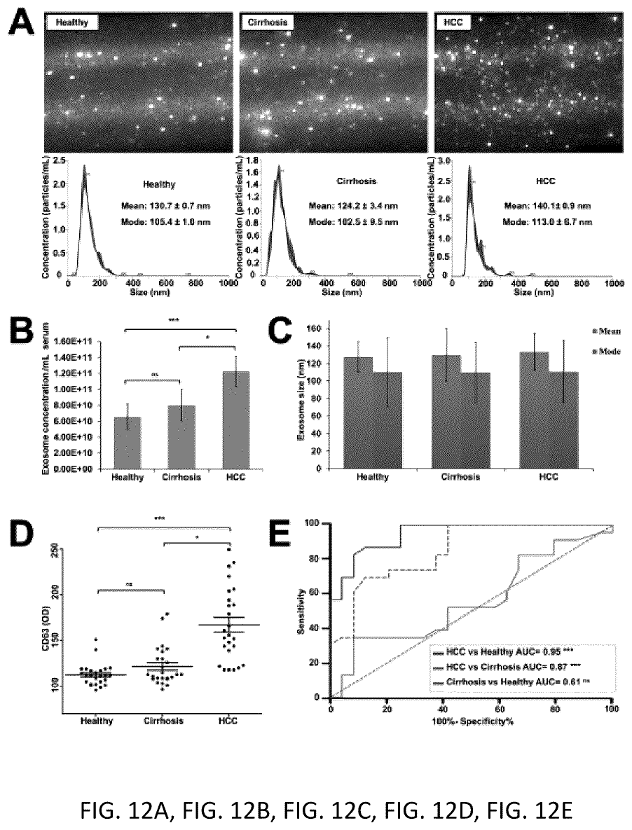

The extent of lysosomal MVB degradation during autophagy is directly related to the quantity of exosomes released. Using serum isolated from a cohort of non-cirrhotic (n=25) and cirrhotic with (n=25) and without HCC (n=25), the relationship between exosome concentration and HCC disease status using NTA was investigated. Exosomes isolated from the serum of each cohort were similar in size distribution and matched the anticipated size distribution for circulating exosomes ranging from 30-150 nm (FIG. 12A). Having confirmed defective autophagy in the HCC tissue, the hypothesis that impaired autophagy results in the increased release of exosomes into peripheral circulation in patients with HCC was examined. As expected, the concentration of exosomes in patients with HCC was significantly higher with respect to both non-cirrhotic healthy controls and cirrhosis without HCC (FIG. 12B). Comparison of exosome size among the groups was not statistically different (FIG. 12C). To verify NTA analysis, the exosome-derived concentration of CD63 (eCD63) tetraspanin was quantified in the purified exosomes from each group by ELISA. In agreement with NTA analysis, eCD63 concentration was significantly elevated in HCC compared cirrhotic and non-cirrhotic controls (FIG. 12D). Receiver operating characteristic (ROC) curves were utilized to determine the accuracy of eCD63 to differentiate HCC from cirrhotic and non-cirrhotic controls. Area under the curve (AUC) was used to compare eCD63 assay performance. An AUC of 0.95 was obtained for eCD63 in differentiating HCC from patients without cirrhosis (P<0.0001), with an AUC of 0.87 in distinguishing HCC from patients with cirrhosis alone (P<0.0001) (FIG. 12E). This data confirms that CD63 exosome content in the plasma accurately identifies patients with confirmed HCC.

v. Diagnostic Performance of Serum eGPC3 for HCC:

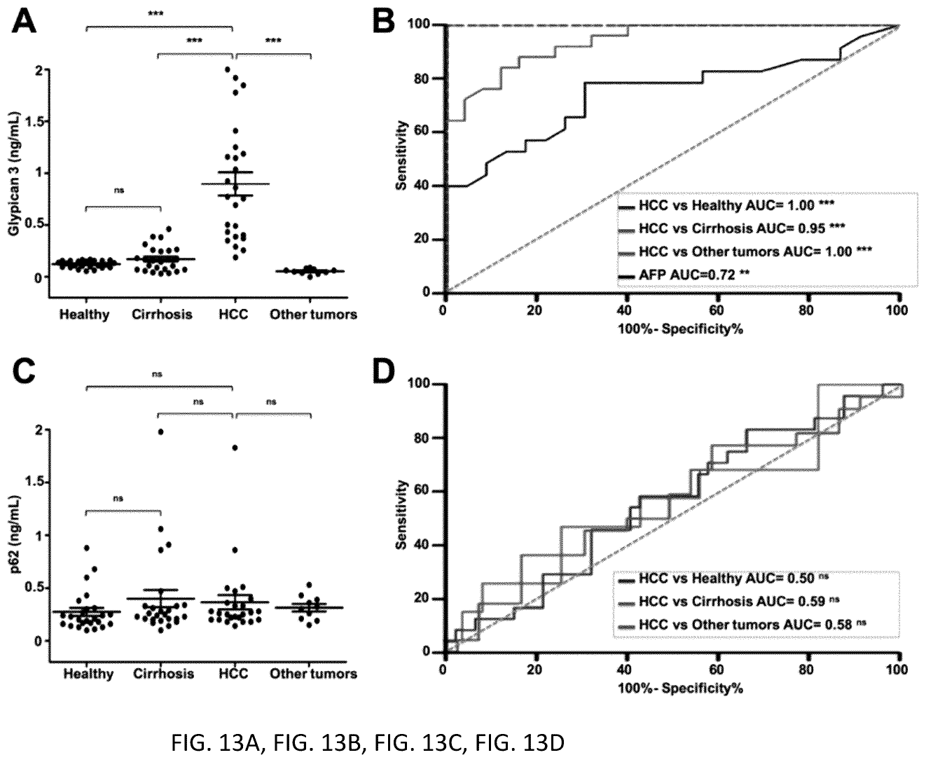

The conventional ELISA assay has proven to be a rapid, high throughput approach for molecular analysis of proteins expressed on the exosomes surface. Using this approach, the levels of GPC3 in serum from patients without cirrhosis (n=25), cirrhosis with (n=25) and without HCC (n=25), as well as serum from patients with malignancy other than HCC (n=10) were characterized. Demographic data for the serum cohort were disclosed in Table 2. Exosome-associated GPC3 was prominently detected in patients with HCC with a small percentage of cirrhotic patients without HCC having GPC3 levels slightly above baseline (FIG. 13A). GPC3 was notably absent in exosome preparations from non-cirrhotic and non-HCC malignancy controls. The GPC3 content in the isolated exosomes was significantly higher in HCC patients compared to healthy control, cirrhosis, and other cancers (P<0.0001). The performance of eGPC3 in detecting HCC was analyzed using ROC analysis to tabulate AUC (FIG. 13B). The eGPC3 level was able to discriminate HCC from cirrhotic patients without HCC (AUC 0.95, P<0.0001) as well as HCC from non-cirrhotic controls (AUC 1.0, P<0.0001) while outperforming AFP (AUC 0.72, P<0.007) in discriminating HCC from cirrhosis without HCC. Exosome-derived p62 (ep62) was similar among all patient groups investigated (FIG. 13C), with ep62 level unable to distinguish disease status in the cohort (FIG. 13D).

TABLE 2
Demographical data of patients with
cirrhosis, HCC, and non-liver cancer

| | Cirrhosis | HCC | Non-liver cancer |
|---|---|---|---|
| | (n = 25) | (n = 25) | (n = 10) |
| Age (years) (mean ± SD) | 61 ± 4.8 | 66.5 ± 8 | 59.4 ± 12.3 |
| Gender (n/%) | | | |
| Female | 10/40 | 7/28 | 5/50 |
| Male | 15/60 | 18/72 | 5/50 |
| Race (n/%) | | | |
| White | 11/44 | 4/16 | 4/40 |
| African American | 5/20 | 8/32 | 5/50 |
| Caucasian | 8/32 | 9/36 | 0/0 |
| Other | 1/4 | 4/16 | 1/10 |
| Cirrhosis Etiology (n/% | | | |
| HCV | 19/76 | 16/64 | N/A |
| ETOH | 3/12 | 1/4 | N/A |
| Other | 3/12 | 8/32 | N/A |
| Serum AFP (median, | 3.6 | 81 | N/A |
| ng/ml) [IQR] | [2.3-8.3] | [12-1089] | |
| Tumor Type (n/%) | | | |
| HCC | 0/0 | 25/100 | 0/0 |
| Prostate | 0/0 | 0/0 | 3/30 |
| Breast | 0/0 | 0/0 | 4/40 |
| Pancreas | 0/0 | 0/0 | 3/30 |

HCC: Hepatocellular carcinoma
AFP: Alpha feto protein,
IQR: Interquartile range 25th-75th percentile,
ETOH: Ethanol,
HBV: Hepatitis B Virus,
HCV: Hepatitis C Virus,
N/A: Non-applicable

vi. Prognostic Value of eGPC3 for HCC Treated with DEB-TACE:

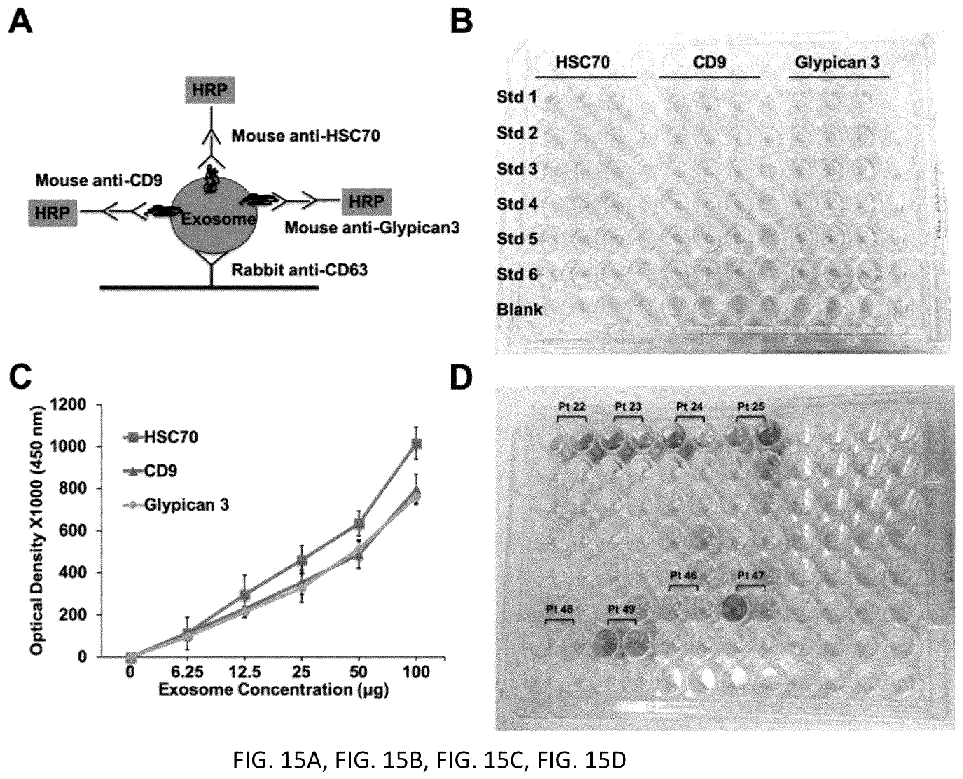

The role of eGPC3 in prognosis of transarterial chemoembolization and whether eGPC3 levels changed in accordance with response to treatment were evaluated. In order to allow facilitate multiplex screening from a small volume serum sample, an affinity ELISA with anti-CD63 capture was utilized to isolate exosomes from the serum (FIG. 15A). Using this assay, multiple aliquots of a single serum specimen can be analyzed for several exosome-associated targets (FIG. 15B and FIG. 15C). Exosome concentration standard curves were developed by serial dilution of exosomes isolated from HCC cell lines. The GD of the ELISA was utilized to analyze eGPC3 content before and after therapy (FIG. 15D).

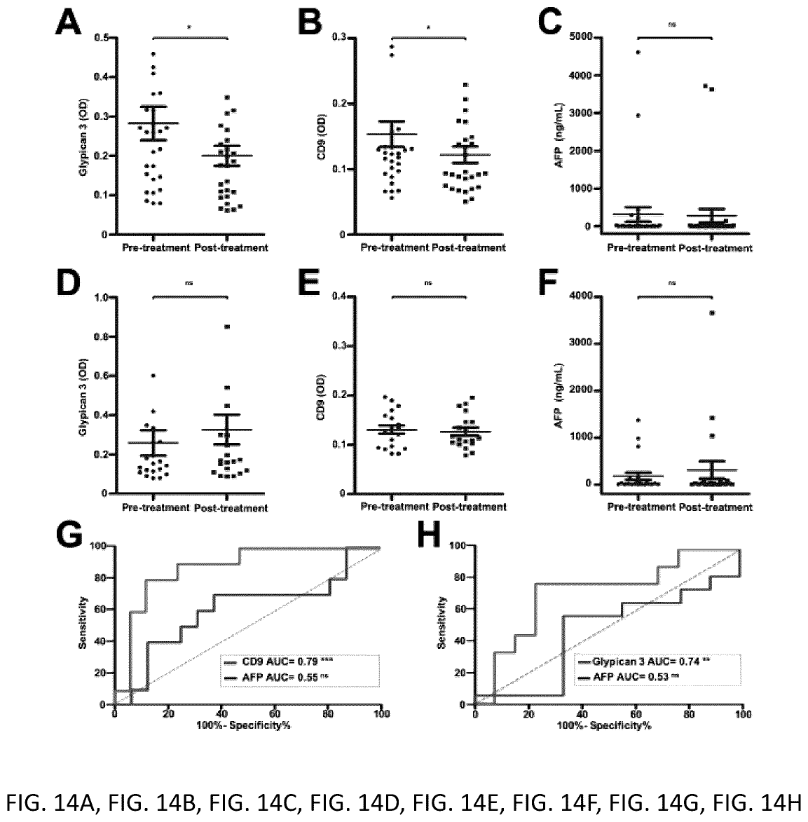

Using the affinity ELISA exosome capture method, paired serum samples from HCC patients receiving first line DEB-TACE were analyzed. Cohort demographics are outlined in (Table 3). After treatment follow-up imaging, patients were grouped based on response to treatment using mRECIST for HCC. Scores were groups in responders (complete or partial response to treatment) and non-responders (stable disease or progressive disease). The eGPC3 and eCD9 content in the bound exosomes was determined in 100 μL serum aliquots and cross-referenced with the clinical serum AFP value at each time point. Of the 43 patients examined, only 32.5% of patients had an abnormal AFP value (AFP>20 ng/mL) at baseline. Patients responding to DEB-TACE were found to have decreased eGPC3 (P=0.038) and eCD9 (P=0.04) at treatment follow-up (FIG. 14A and FIG. 14B). As anticipated, the post-treatment AFP level was not yet significantly changed with respect to baseline when considering all AFP values or only AFP values above the normal threshold (P=0.84) (FIG. 14C). In contrast, there was no reduction in eGPC3 levels in non-responders, with a trend toward increasing eGPC3 (FIG. 14D). The eCD9 content and serum AFP level were also not significantly altered post-treatment in the non-responder group (FIG. 14E and FIG. 14F). ROC curve analysis of change in eCD9 content of the CD63-bound exosomes was found to be more predictive for response to treatment than change in AFP. In the responder group there was a mean decrease of 21.6% in eCD9 post-treatment with ROC analysis sensitivity of 72.7% and specificity of 88.2% (FIG. 14G). A mean increase in eGPC3 of 11.9% was present in non-responders, with a ROC analysis sensitivity of 77.7% and specificity of 78.5% (FIG. 14H). Collectively, these results show that changes in exosome concentration, tetraspanin fraction, and GPC3 content may also have implications in correlating treatment response and disease progression.

TABLE 3
Demographic data of patients with HCC undergoing DEB-TACE

| | Total (n = 43) | |
|---|---|---|
| Age (years) (mean ± SD) | 60.1 ± 6.1 | |
| Gender (n/%) | | |
| Female | 18/41.9 | |
| Male | 25/58.1 | |
| Race (n/%) | | |
| Caucasian | 34/79.1 | |
| African American | 3/7 | |
| Other | 6/14 | |
| Cirrhosis Etiology (n/%) | | |
| HCV | 23/53.5 | |
| HCV + ETOH | 6/18.6 | |
| NAFLD | 8/18.6 | |
| ETOH | 4/9.3 | |
| HBV | 1/2.3 | |
| AIH | 1/2.3 | |
| MELDNa score median [IQR] | 11 | [9-16] |
| Number of lesion median (min-max) | 1 | (1-3) |
| Largest lesion diameter (cm) (mean ± SD) | 2.8 ± 0.7 | |
| Cumulative burden size (cm) (mean ± SD) | 3 ± 0.9 | |
| Pre-treatment AFP (ng/ml) median, [IQR] | 10 | [5.4-31] |
| Post-treatment AFP (ng/ml) median, [IQR] | 11 | [4.5-36] |

| | |
|---|---|
| Objective Response | |
| Present (n/%) | 24/55.8 |
| Absent (n/%) | 19/44.2 |

AFP: Alpha feto protein,
IQR: Interquartile range 25th-75th percentile,
AIH: Autoimmune hepatitis,
ETOH: Ethanol,
HBV: Hepatitis B Virus,
HCV: Hepatitis C Virus,
NAFLD: Non-Alcoholic Fatty Liver Disease

3. Discussion

Autophagy functions as a tumor suppressor in hepatocytes promoting endo-lysosomal and autophagosome-lysosomal degradation, which provides protection against cellular transformation. Impaired autophagy has been linked to several human diseases such as neurodegenerative disorders, microbial infection, aging, and many types of cancer including HCC. Impaired autophagy leads to the accumulation of p62, and polyubiquitinated proteins. Although highly expressed in the fetal liver, GPC3 is not expressed in the healthy adult liver but is reactivated within the HCC lesion. A variety of growth factor receptors, including some linked to oncogenic signaling pathways, are controlled by GPC3. GPC3 is also involved in endocytosis, degradation of MVBs, and exosome release. Impaired autophagy inhibits degradation of GPC3 and MVBs, providing the mechanism through which GPC3 activates oncogenic signaling pathways in HCC.

In this study, increased expression of p62 and GPC3 within the HCC lesion independent of underlying cirrhosis etiology is shown. Increased p62 and GPC3 expression was also observed in HCC cell lines compared to normal hepatic cells, supporting an autophagy-deficient status in HCC cells. Autophagy-deficient HCC cells secrete more exosomes as compared to autophagy-sufficient cells. GPC3 expression was restricted to exosomes isolated from HCC cell lines confirming eGPC3 status can differentiate malignant from benign liver cells. This finding was then translated to HCC surveillance to test the hypothesis that serum exosome concentration and eGPC3 levels can serve as an HCC surveillance biomarker in cirrhotic patients.

Exosome isolation/purification remains a major barrier to clinical implementation of exosome-based diagnostic biomarkers. Several isolation strategies have been utilized for biochemical and morphological characterization of exosomes. Ultracentrifugation is the gold standard for exosome isolation, although from a translational perspective this approach lacks high throughput capability. Molecular-based exosome isolation strategies offer a translatable, high throughput alternative with concessions in exosome integrity and purity that may interfere with downstream analysis. This is addressed by using immunoaffinity capture to isolate circulating exosomes from the serum. Using an approach disclosed herein, a conventional, affinity ELISA-based exosome assay was developed for multiplex analysis of protein expression in a high throughput format.

The results disclosed herein demonstrate that total exosome concentration is significantly elevated in patients with HCC meeting diagnostic criteria compared to subjects with cirrhosis and non-cirrhotic controls. The size of the exosomes however was not significantly different among the groups. The exosome concentrations obtained using CD63 ELISA strongly correlated with matched samples analyzed using the NTA-based approach to exosome quantification. Although the exosome concentration between cirrhotic and non-cirrhotic controls did not reach significance, eCD63 levels were elevated in patients with cirrhosis. This finding was anticipated given the role of autophagy-deficiency in malignant transformation of HCC and could provide a rationale for increased surveillance for suspected pre-malignant changes in the liver. The studies disclosed herein demonstrate that serum eGPC3 accurately distinguishing patients with HCC from non-cirrhotic controls, cirrhotic controls, and individuals with other non-HCC malignancy. GPC3 exosome status more accurately confirmed HCC diagnosis compared to serum AFP. In addition to improved diagnostic performance, exosome concentration and eGPC3 level were also prognostic for response to tumor-directed locoregional chemotherapy. Decreased eGPC3 level and exosome concentration after locoregional chemotherapy were associated with an objective response to treatment. Serum ep62 level did not differ among analysis groups, suggesting this marker may not be applicable for HCC detection.

These results indicate that exosomal glypican-3 is a promising HCC biomarker that can accurately detect HCC as well as treatment response. The methods disclosed herein for the detection of GPC3 is specific that can be adapted to any laboratory using a small volume of serum samples. The exosomal GPC3 detection has a very high predictive values that correlate with ultrasound evaluation of HCC treatment.

### C. Example 3: A Single-Step Immunocapture Assay to Quantify HCC Exosomes Using the Highly Sensitive Fluorescence Nanoparticle-Tracking Analysis

1. Introduction

Primary liver cancers, including hepatocellular carcinoma (HCC) and intrahepatic cholangiocarcinoma (ICC), are the leading cause of cancer-related death worldwide. HCC accounts for 80-90% of primary liver cancer and most often develops in the context of cirrhosis due to viral or non-viral chronic liver disease. The hazard ratios for developing HCC are higher in patients with chronic viral hepatitis due to Hepatitis C (HCV) or Hepatitis B (HBV). HCV infection can be cured with direct-acting antivirals (DAAs) which have begun to decrease the incidence of HCV-HCC. However, the most prominent non-viral etiologies, alcoholic and non-alcoholic fatty liver disease, cause hepatic steatosis that can progress to cirrhosis and HCC. Recent projections estimate HCC incidence will increase worldwide among the aged population with metabolic syndrome, diabetes, and toxic environmental exposure. An alarming majority of HCC is diagnosed at an advanced stage and accompanied by a dismal prognosis with limited treatment options. Driven by a high mortality rate, HCC is now the third-leading cause of cancer-related death. The current American Association for the Study of Liver Diseases guideline recommends biannual surveillance in patients with cirrhosis using liver ultrasound±serum alpha-fetoprotein (AFP) for early detection of HCC. The sensitivity of this AFP-based HCC surveillance is between 60% and 70%, with a specificity of 90%. There remains an urgent need to expand HCC biomarkers to improve early detection, access to curative treatment options, and overall outcomes.

Extracellular vesicles (EV) are nanometer size, closed lipid-bilayer vesicles naturally released by most cells in the body. Recent studies have shown that EV release plays many important roles in facilitating intracellular communication in both normal and pathological conditions. EV release occurs through outward budding of the plasma membrane or multivesicular body fusion with the plasma membrane. EV shedding plays a critical role in maintaining homeostasis, cell-to-cell communication, inflammation, aging, metabolic diseases, neurological diseases, and cancer. The EV release was also demonstrated in plants and bacteria indicating this process is evolutionarily conserved. Each cell type in our body fine-tunes EV biogenesis with a specific lipid, protein, and nucleic acid composition depending on its metabolic state. EV release is controlled by autophagy and intracellular membrane trafficking and helps maintain normal cellular function. In stressed conditions, EV release can signal disease onset and organ dysfunction.

In addition to releasing hepatic EVs, the liver plays a central role in clearing EVs from systemic circulation. Hepatic EVs quantification has emerged as a promising prognostic indicator for early assessment of chronic liver disease, and progression to cirrhosis, and HCC. ASGPR1 is a transmembrane molecule expressed in the sinusoidal and basolateral membrane of hepatocytes, but not in the bile canalicular apical membrane. The ASGPR1 expression has been used as a marker to assess the polarized EV release from the liver A recent study shows that the integrated stress response (ISR) and hepatic adaptive plasticity caused by HCV replication led to impaired autophagy and increased EV release in the cell culture model. It was demonstrated that HCC development in the cirrhotic liver is accompanied by impaired autophagy and increased EV shedding (microvesicle and exosomes). This data is consistent with other reports indicating that cancer cells secrete significantly higher amounts of exosomes and microvesicles than normal cells, which could be harnessed as a minimally invasive biomarker for cancer diagnosis. However, isolating and quantifying organ-specific exosomes has created a significant hurdle to establishing causal relationships between EV biology and underlying disease. The ability to accurately quantify specific exosome subsets in the blood would help establish and distinguish EV-based non-invasive diagnostics in HCC surveillance. Several methods have been utilized for the exosome quantification, including enzyme-linked immunosorbent assay (ELISA), Western blotting, flow cytometry, tunable resistive pulse sensing (TRPS), electron microscopy, dynamic light scattering (DLS), microfluidics, surface plasmon resonance (SRR), and single particle interferometric reflectance imaging sensor (SP-IRIS). These techniques require specialized, costly instrumentation, technical expertise, and time-consuming methodologies complicating their applications in the resource-limited clinical laboratory setting. The clinical impact of EV-based diagnostics and exosome-based biomarkers has therefore been limited, largely due to the lack of a standardized, well-established technology to accurately quantify biomolecules associated with EVs. NTA is the most sensitive instrument that allows the analysis of EVs on a single particle level in light scatter mode (LSM) and fluorescent mode (FM). This equipment directly provides the video image of EVs in motion and simultaneously measures their size and concentrations using the Stokes-Einstein equation.

In this study, an optimized, single-step immunocapture assay combined with mini-column purification is optimized for microliter scale quantification of HCC-derived exosomes in their native state using NanoSight NS300 in fluorescence mode (F-NTA). The ability of the F-NTA method to quantify exosomes was validated by using synthetic fluorescence beads, HCC cell line-derived EVs, and tumor-derived EVs from patients with confirmed HCC. F-NTA assay performance was optimized using antibodies targeted to GPC3 and AFP alone and in combination. HCC-derived exosome quantification by F-NTA was directly compared with magnetic bead-based affinity flow detection methods. The results indicate that F-NTA has a superior sensitivity for detecting HCC-derived exosomes and is scalable to microliter-level sample volumes. F-NTA distinguished patients with confirmed HCC from malignancy-free cirrhosis as well as healthy controls. In culture supernatant, F-NTA had a detection limit as low as 10² HCC-derived exosomes per tumor cell. The concentration of HCC exosomes in the serum detected by F-NTA shows strong correlation with tumor size assessed by MRI. Serum HCC-derived exosome levels were calibrated with MRI-based assessments of HCC burden to demonstrate compatibility as a diagnostic assay.

2. Materials and Methods

i. Cell Culture and Reagents:

Human hepatoma cell line (Huh7) (CAR-STC-ZP51) and the human cholangiocarcinoma cell line (HuCCA-1) were purchased from Creative Bioarray (CSC-C6896J) (Shirley, NY) and cultured in DMEM high glucose with 10% exosome-free fetal calf serum (FBS), L-glutamine, 1 mM sodium pyruvate, and 0.1 mM non-essential amino acids with 1% penicillin and streptomycin. At confluence, the cells were split in a 1:3 ratio. Exosome-free fetal bovine serum was purchased from Thermo Fisher, Waltham, MA (A2720803).

ii. EV Purification from Huh7 and HuCCA-1 Cell Culture Supernatant:

Cells were cultured in exosome-depleted media for three days before EV analysis. Briefly, cellular debris was removed from the culture supernatant by centrifugation at 2000 g for 30 minutes. Cleared supernatants were transferred to a new tube and mixed with 0.5 volumes of total EV isolation reagent (4478359) from Invitrogen Carlsbad, CA. Tubes were incubated at 4° C. overnight. The following day samples were centrifuged at 10,000 g for one hour at 4° C. The resulting supernatant was carefully removed, and the EV pellet was resuspended in PBS and stored at −20° C. until further analysis.

iii. Nanoparticle Tracking Analysis (NTA):

EV concentration and size distribution were determined using NanoSight (Model NS300-NTA3300, Malvern Panalytical, Worcestershire, UK) equipped with a 532 nm green laser and a 565 nm long-pass filter for fluorescence detection. Technical triplicate measurements in FM and LSM were performed for each serum or cell culture-derived sample used in this study. Brownian motion of EVs is visualized in real-time while liquid-state light scattering properties are utilized to measure EV concentration and size. A syringe pump enables the free movement of particles in the tube. EVs in suspension are passed through a flow chamber and illuminated by a laser source to analyze total particle concentration by the NTA software as particle/ml (v3.2, Malvern Panalytical, Worcestershire, UK). Initially, FM measurements were completed and sequentially LSM measurements were done to avoid the ‘photobleaching’ effect. Either in FM or LSM, exosome concentration analysis was performed using a 30-second recording per individual replicate. A washing step was performed between each measurement using ultrapure particle-free water. The fluorescence signal generated from the particles is recorded with a digital camera. Representative images were captured from these video records. The NTA instrument has a limited dynamic range for particle concentration (10⁴-10⁸ particles/ml). Therefore, all the samples were diluted to a maximum concentration of 1×10⁸ particles/ml to obtain accurate concentration measurements within the instrument's detectable range.

Measurements of LSM were performed with camera level settings (12-14) and 15-16 to measure in FM. If there was a noise that would affect the analysis quality, the measurement was repeated by going to the next dilution step and the dilution rates were recorded. The syringe pump flow rate was set to 25 frames/second, and the detection threshold was set up to 5. Optimal camera and detector settings were fixed and remained constant for patient-derived serum sample analysis. Phycoerythrin (PE)-conjugated anti-human CD9, AFP, and GPC3 antibodies were used for the fluorescence detection of EVs. PE-labeled monoclonal rabbit and mouse antibodies against GPC3 (sc-390587 PE), AFP (sc-8399 PE), CD9 (sc-13118 PE), ARF6 (sc-7971 PE), and ASGPR1 (sc-166633 PE) were purchased from Santa Cruz Biotechnology (Dallas, TX). Antibody dilutions were optimized through antibody titration experiments using HCC-derived exosomes purified from Huh7 cells. EV samples were incubated with GPC3 antibody at 1:25 (v/v), AFP antibody at 1:100 (v/v) in PBS, CD9 antibody (1:25), ARF6 antibody (1:25), ASGPR1 antibody (1:25) with 1% BSA overnight at 4° C. with agitation. The next day EVs were purified by size exclusion chromatography and immediately processed for NTA analysis. The assay performance to detect HCC exosomes in the serum using multiple dilutions of GPC3 and AFP antibodies was determined. For this purpose, 10 μl of normal serum or HCC serum was diluted in 100 μl of PBS with 1% BSA incubated with a diluted antibody. The next day, samples were diluted in 1 ml of ultrapure water and examined by NTA. The dilution level with minimal background serum signal compared to the positive detection signal was selected.

iv. Quantification of Membranous Particles by NTA:

Serum samples (10 μl) and isolated exosomes from Huh7 cell culture were diluted in 100 μl of PBS. Lipophilic membrane dye (Cell Mask™ Plasma Membrane Stains—C10046, CMDR, ThermoFisher, USA) was diluted in PBS at a proportion of 1:1000. A hundred microliter of diluted dye was mixed with 100 μl of diluted samples and incubated for two hours in a dark room with ambient temperature. The entire staining mix was diluted with up to 1 ml of PBS. The labeled EVs were purified from a free dye by passing through the Sephadex G-200 column. The percentage of membranous EVs was determined using the ratio of particle concentrations measured in FM and LSM.

v. Serum Cohort Study:

Human specimen studies were conducted following the ethical guidelines set forth by the Declaration of Helsinki. Isolated serums from patients with confirmed HCC (n=21) were obtained from a single-center, prospective study. The study inclusion criteria required an HCC diagnosis based on the Liver Imaging Reporting and Data System (LI-RADS) and/or biopsy in accordance with current American Association for the Study of Liver Disease guidelines. Informed consent was obtained from patients following confirmed HCC diagnosis. Among these patients, 10 out of 21 with an MRI-based diagnosis and serum AFP at the time of diagnosis were selected for the biomarker-MRI verification study. Stage F4 cirrhosis serum was obtained from patients with a diagnosis of non-alcoholic steatohepatitis (NASH) (n=20). The cirrhotic patients who had available imaging and AFP levels were enrolled in a radiological validation study (n=10). Healthy control serum from patients with no existing liver disease was obtained from the Tulane Department of Clinical Pathology Laboratory (n=10) after age and sex matching against the cirrhosis and HCC cohorts.

vi. Single Step Immunocapture of Serum HCC Exosomes:

Isolated serum aliquots of 10 μL across all patient cohorts were diluted in 100 μL of PBS with 1% BSA. PE-labeled antibodies to AFP (1:50) and GPC3 (1:25) were added to the diluted serum samples and incubated overnight at 4° C. with agitation. Samples were then diluted to 1 ml with ultrapure particle-free water. Fluorescence-labeled, antibody-coated HCC exosomes were separated from unbound PE-antibodies by size-exclusion column chromatography to improve isolation efficiency and avoid denaturing the isolated EVs. Sephadex G-200 (‘hand-made gel column’, A 50120, Sigma, Burlington MA), Bio-Spin® Chromatography Columns (7326008 Bio-Rad, Hercules, CA), Cell guidance Exo-Spin™ mini columns (EX03-50), and Amersham MicroSpin G-50 Columns (27533001, Cytiva, Marlborough, MA) were used for this step. The columns were washed three times with PBS and then the fluorescence-labeled, immunocaptured HCC exosomes were loaded onto the column. The solution was passed through the column until the entire 1 mL flow-through volume was collected to completely remove the unbound PE-conjugated antibodies. Samples were diluted 1:10 before size and concentration analysis using F-NTA.

vii. Analysis of HCC Exosomes by Immunomagnetic Bead-Based Affinity Flow Cytometry:

Streptavidin-coated magnetic beads (10608D) were purchased from Invitrogen (Carlsbad, CA). Beads were prewashed in PBS and incubated with biotinylated antibodies for one hour. Biotinylated antibodies to CD63 (353018) were used at a proportion of 4 μg/10⁷ of magnetic beads (Bio Legend, San Diego, CA). Antibody-conjugated beads were washed twice using PBS with 1% BSA to block the nonspecific bindings sites. The beads were incubated with 100 μl of cell culture-derived EVs overnight at 4° C. The following protocol was used for immunomagnetic separation of EVs in human serum. Serum samples were centrifuged briefly at 2000 rpm for 5 minutes to remove debris. A 10 μL aliquot of serum was diluted in 100 μl PBS and incubated with immunomagnetic beads for native EV capture. The next day, the EV-bead complex was kept in a magnetic stand and washed three times using 0.5 ml of PBS with 1% BSA. Following this step, the EV-bead complex was incubated with fluorescence-labeled antibodies diluted in 100 μl PBS 1% BSA for one hour at room temperature. Beads were washed three times with 0.5 ml of PBS 1% BSA and then analyzed on a Becton-Dickinson flow cytometer (BD FACS Celesta) using BD FACSDiva Software 6.0 (San Jose, CA). Advanced flow cytometry analyses and graphical output were performed using Flowing Software version 2.5.1 (Turku Bioscience, Turku, Finland).

viii. Statistical Analysis:

Statistical analysis was performed using Prism software version 8 (GraphPad Software, Inc., La Jolla, CA, USA). All experiments were performed 3 independent times with fresh cultures of cells each time to obtain 3 replicates. The variables were investigated using visual (histograms, probability plots) and analytical methods (Kolmogorov-Smirnov/Shapiro-Wilk tests) to test for normal distribution. Continuous variables were shown as mean and standard deviation (SD) or median and 25^{th}-75^{th} quartiles (IQR-interquartile range) according to their distribution pattern. Results were presented The Kruskal-Wallis test was used to compare exosomal concentration size and expression of GPC3, ARF6, and CD9 in patient serum cohorts. A value of P<0.017, calculated by Bonferroni correction, was considered statistically significant when comparing the three patient groups. The Mann-Whitney U test was used to compare the medians between the study subgroups. Spearman correlation analysis was used for investigating the relationship between lesion size and GPC3+ve exosome concentrations. Sensitivity, specificity, and cut-off values were calculated for discriminating HCC and non-HCC cases using receiver operating characteristic curve (ROC) analysis. The statistical significance was shown as * p<0.05, ** p<0.01, *** p<0.001.

3. Results

i. Establishing a Workflow for Quantifying HCC Exosomes in Serum:

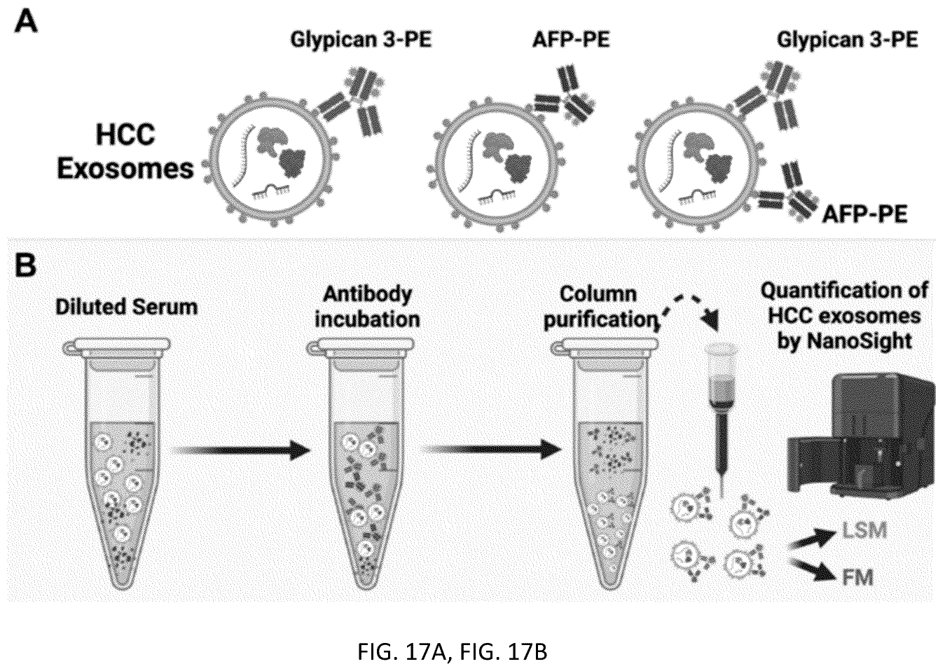

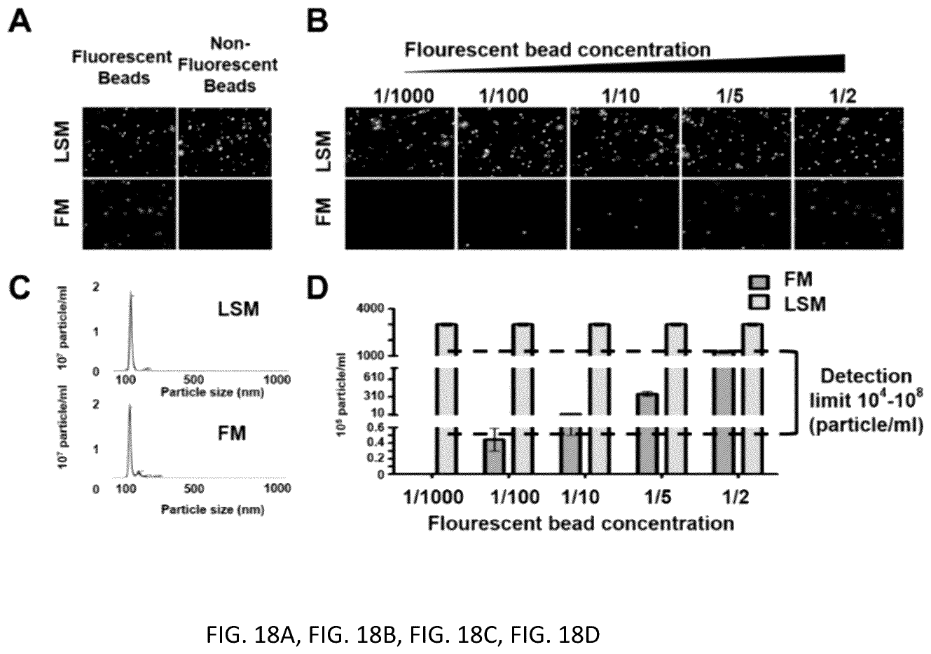

Quantification of EVs isolates released by tumor cells will offer a great advantage for monitoring early cancer growth, tumor size, and treatment response. A fast and easy technique for scalable purification of EVs was developed that uses a combination of immunocapture, and size exclusion chromatography followed by concentration measurements by fluorescence nanoparticle tracking analysis (F-NTA) summarized in FIG. 17. The serum EV immunocapture and size exclusion chromatography were used to separate the fluorescence-labeled EVs from the unbound fluorescence antibodies. The F-NTA protocol was optimized to quantify circulating HCC-derived exosomes in isolated serum after immunocapture with PE-conjugated antibodies against AFP or GPC3. The F-NTA measures the fluorescence-positive EVs in the red fluorescence channel and total EVs in the light scatter channel. Initial experiments were carried out to determine the detection range of F-NTA using Synthetic Beads. F-NTA performance was established by serial dilution using fluorescent-labeled beads (FIG. 18). Unlabeled fluorescent beads were detected in light scatter mode (LSM) with no autofluorescence signal detected in FM. Serially diluted fluorescent-labeled beads were similarly detected in the LSM mode and detected in FM (FIGS. 18A and B). The median particle size for both unlabeled and fluorescent-labeled beads was correctly calculated in both LCM and FM, respectively (FIG. 18C). F-NTA detection range was determined using a fixed concentration of unlabeled beads spiked with varying concentrations of synthetic fluorescence beads with dynamic particle imaging recorded in FM and LSM (FIG. 18D). F-NTA detection limits for fluorescence-labeled beads ranged from 10⁴ to 10⁸ particles/ml which established the optimal target dilution for subsequent experiments.

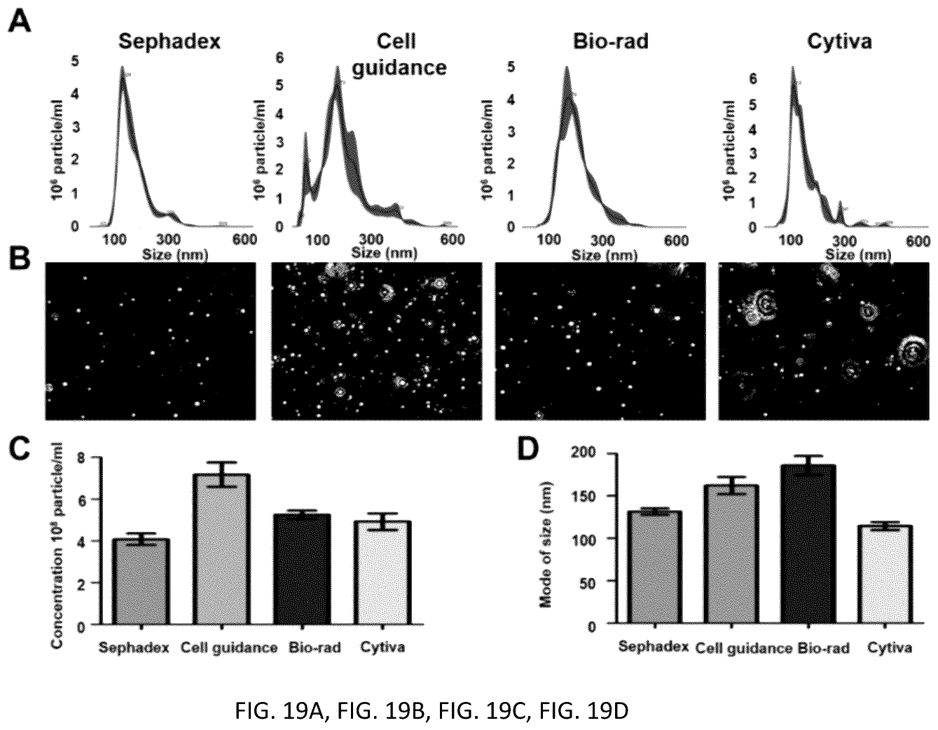

The size exclusion chromatography step was optimized by comparing four different approaches including four commercially available laboratory protocols. To identify the optimal recovery strategy which preserved exosome morphology, the mean concentration and mode size for each column preparation was analyzed in LSM (FIG. 19). Analysis of the standard and model of vesicle size and video images of exosome light scattering show that all columns produced excellent exosome yield since they all generate exosome numbers 1×10⁸ per ml. As shown the yield and purity of EVs varies among different column isolation. The recovery and size of vesicles varied among different column purification approaches, with more heterogenous particles found in the Cell Guidance and Cytiva gel filtration column (FIG. 19A), which was confirmed in the video images of particles (FIG. 19B). The size measurement indicates bigger non-vesicular particles or protein aggregates could be contributing mean diameter to a bigger size. Since the Sephadex G-200 column purification provided the optimal purification (FIG. 19C and FIG. 19D) it was selected for subsequent analyses of exosome purification.

ii. Purity, Population Diversity, and Liver Specificity Check of Column Purified EVs by F-NTA:

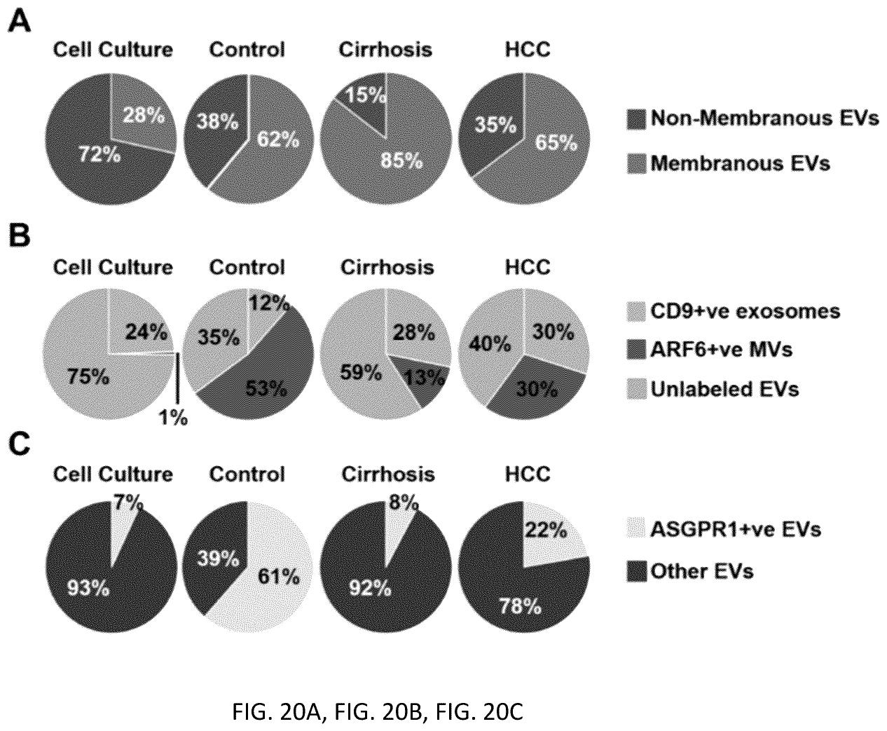

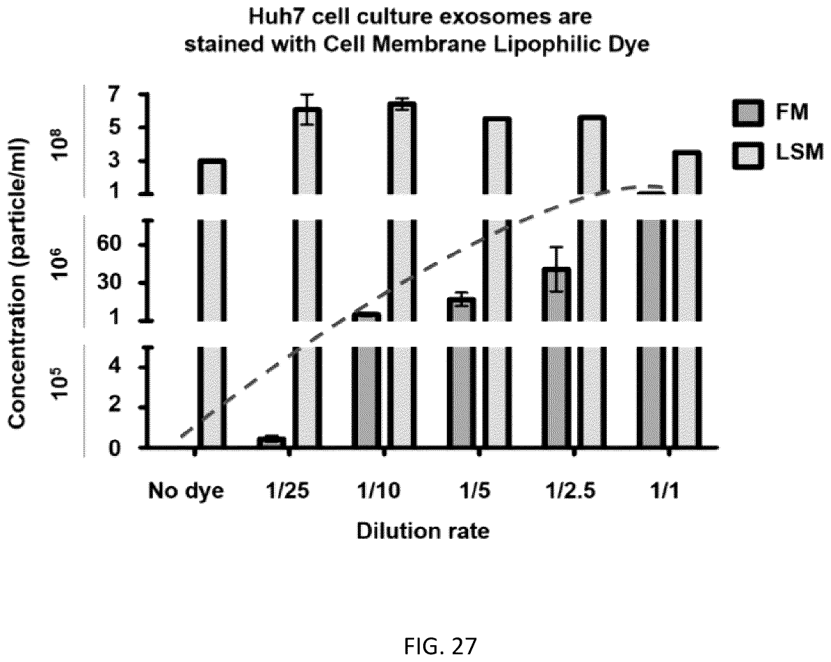

The NTA does not discriminate different biological vesicles such as membrane and non-membrane particles such as protein aggregates or salt precipitate. An experiment was designed that distinguishes membranous and non-membranous EVs after labeling them with a lipid-permeable fluorescent dye. The dye was diluted to a concentration of 1:1000 and then a dose-dependent dye titration was performed using isolated EVs from Huh7 cells. The labeled EVs and unbound dye were separated by column purification. The concentration of dye that shows maximum labeling was determined by F-NTA used in the serum studies (FIG. 27). Serums of healthy individuals, patients with cirrhosis, and HCC were labeled with lipophilic red dye for two hours, and EVs were column purified. The concentration of fluorescence-positive membranous particles and total particle counts in each sample was determined by NTA in FM and LSM. The data clearly show that column purification of EVs has a purity of >80% (FIG. 20A). The immunoreactivity of EVs was tested after column purification to confirm the suitability of this method for diagnostic use. For this purpose, EV population diversity following immunocapture and gel-filtration assay was confirmed using PE-conjugated antibodies specific to microvesicles (ARF6) and exosomes (CD9). This analysis shows that Huh7 cells release several subtypes of EVs with only 1% of EVs positive for the microvesicle marker ARF6 and 24% of EVs positive for the exosome marker CD9 (FIG. 20B). The majority of EVs (>75%) secreted by the Huh7 cells were negative for both the microvesicle and exosome marker. A similar EV population analysis was performed using 10-μl of serum from a healthy individual, a cirrhotic patient, and a patient confirmed for HCC. In the healthy control sample, only nearly 12% of the isolated EVs stained positive for the exosomal marker with 53% showing positive for the microvesicle marker and the remaining 35% showing negative for exosome and microvesicles. However, EV population diversity was found to be different in cirrhotic patients with or without HCC. EV isolated from patients with cirrhosis and HCC were nearly 2-3-fold enriched in exosome positive fractions (28% and 30%, respectively) with microvesicle fractions of 13% in patients with cirrhosis and 30% in HCC. Of note, there was no difference in the total number of particles isolated between equal volumes of Huh7 cell culture supernatant and patient subgroups. The next set of experiment was performed to test whether peripheral blood sample contained adequate amount of liver-derived vesicles. Normalized distribution of the liver-derived ASGPR1+ve EVs in cell culture, normal healthy serum, cirrhosis, and HCC serums are presented in FIG. 20C. EVs isolated from Huh7 culture showed low positivity of ASGPR1. Normal healthy individuals release high amounts of ASGPR1+ve EVs and the production decreased in liver cirrhosis and HCC.

iii. F-NTA Assay Calibration Using EVs Isolated from Huh7 Cell Culture:

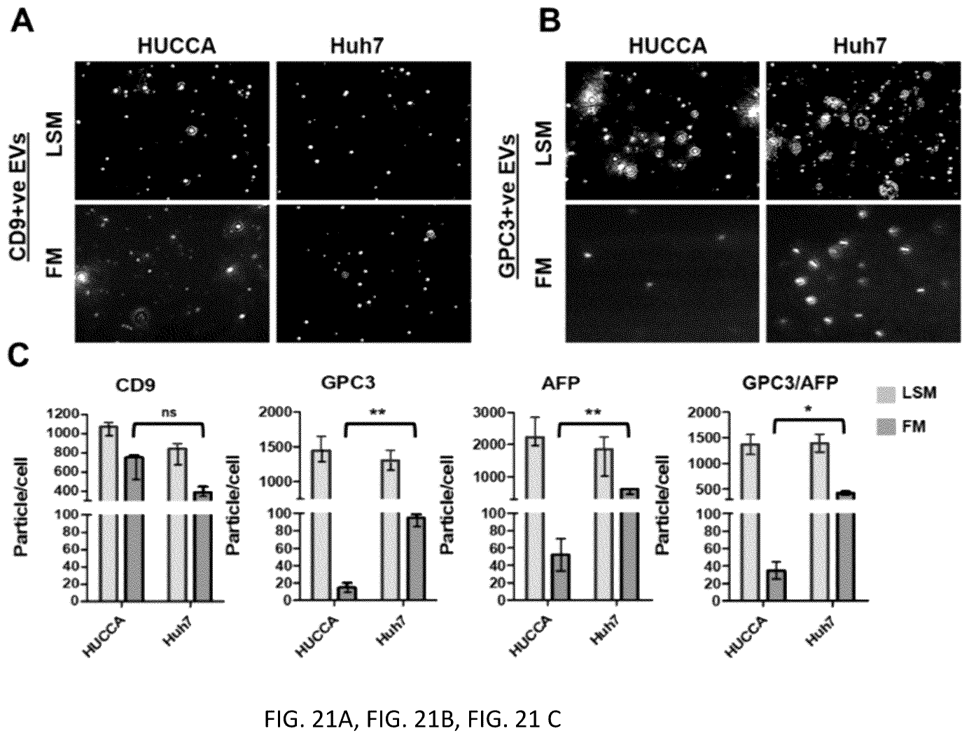

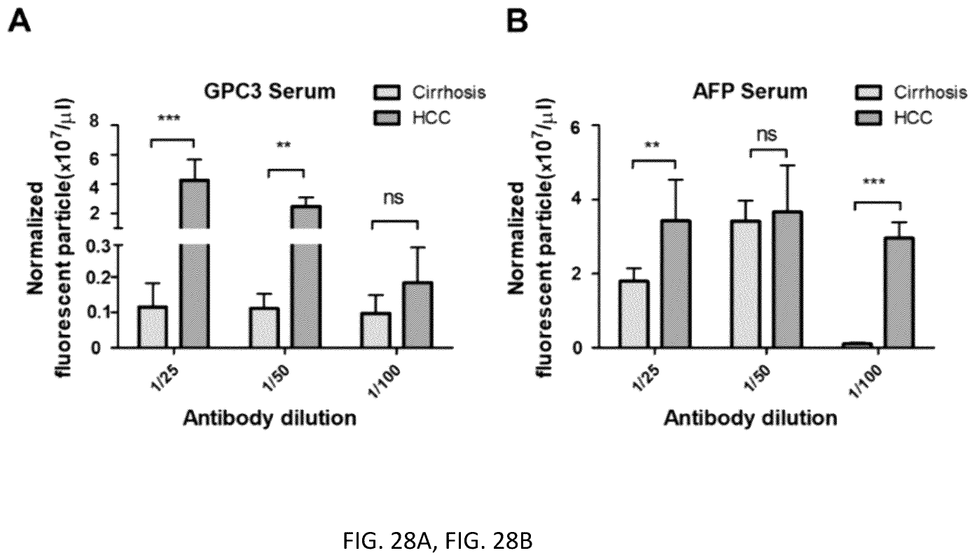

The first sets of experiments determined the antibody dilutions that show optimal detection with minimum non-specific background signal. It was found that the GPC3 antibody at 1:25 and AFP (1:100) produced optimum quantification of the HCC exosomes (FIG. 28). The second set of analyses was performed to compare HCC and non-HCC exosomes for the expression of known HCC markers. Exosomes were isolated from Huh7 and HuCCA-1 cell cultures by precipitation and immunocapture assay using PE-labeled antibodies to AFP or GPC3 alone and in both antibodies in combination (FIG. 21). Exosomes from Huh7 and HuCCA-1 cells immunocaptured with PE-labeled CD9 antibodies were used as the internal control. The antibody reaction was compared between HCC and non-HCC cell lines. Representative LSM and FM images of light scattering, CD9+ve, and GPC3/AFP+ve exosomes isolated from Huh7 and HuCCA-1 culture supernatants are shown in FIGS. 21A and B. The estimated exosome shedding per tumor cell was determined by normalizing the cell number in the culture. Both LSM and FM particle concentrations were higher in HuCCA-1 cell culture samples than in Huh7 cell culture samples in the CD9 experiment (For LSM 1068±80.4 vs. 843±88.7 particle/cell p=0.03 and FM 751±38.1 vs. 389.3±101.1 particle/cell p=0.004). Due to this reason, fluorescent CD9+ve particle ratios were found equivalent in samples from Huh7 cells and HuCCA-1 cells. The fluorescent positive HCC particle concentration is significantly higher in Huh7 exosomes than exosomes from HuCCA-1 cells in terms of GPC3, AFP, and a combination of GPC3 and AFP. Total particle concentration and yield of particles between Huh7 and HuCCA-1 were comparable in these experiments (p>0.05). The per-cell shedding frequency for GPC3+, AFP+, and GCP3+AFP+ EVs was higher in Huh7 cells compared to HuCCA-1 (FIG. 21C). This analysis allowed us to compare the average exosome release between Huh7 and HuCCA-1 cells. HuCCA-1 cells release more exosomes compared to HCC cells. However, this analysis resulted in the detection of more GPC3+ve HCC exosomes in Huh7 cells compared to HuCCA-1 cells (100 GPC3+ve exosome per Huh7 as compared to 10 GPC3 positive exosome per HuCCA-1 cells). The F-NTA analysis showed 200 AFP-positive exosomes in HuCCA-1 cells compared to 500 AFP-positive exosomes in Huh7 cells. AFP and GPC3 antibody combination captured more HCC exosomes in Huh7 cells than in HuCCA-1 cells. The number of fluorescence-labeled particles called HCC exosome per cell was found to be in the range of 1-200 particles/cells when AFP and GPC antibodies were used in combination.

iv. Quantification of HCC-Derived Exosomes in the Clinical Samples:

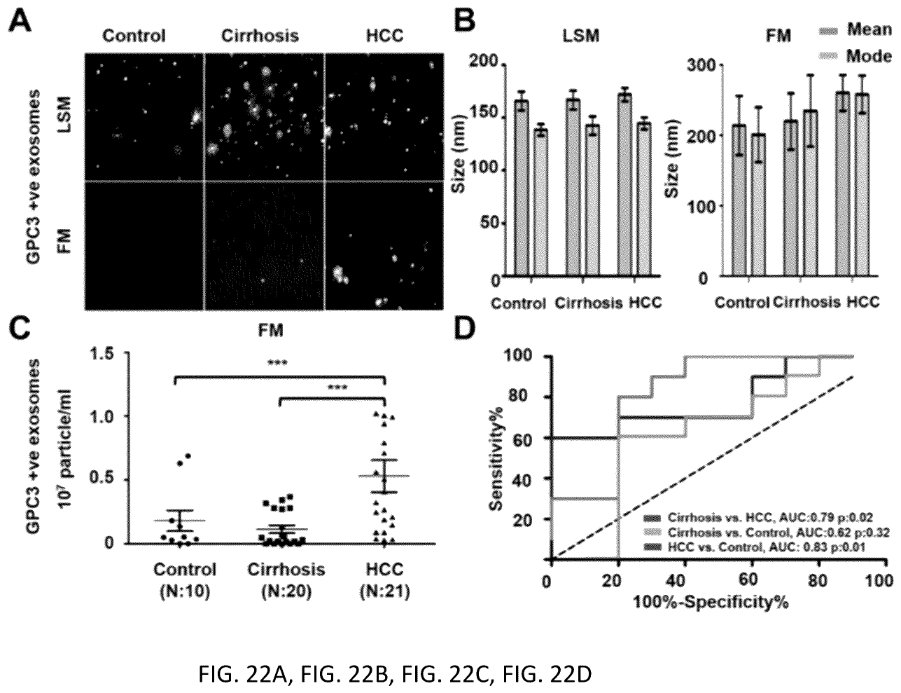

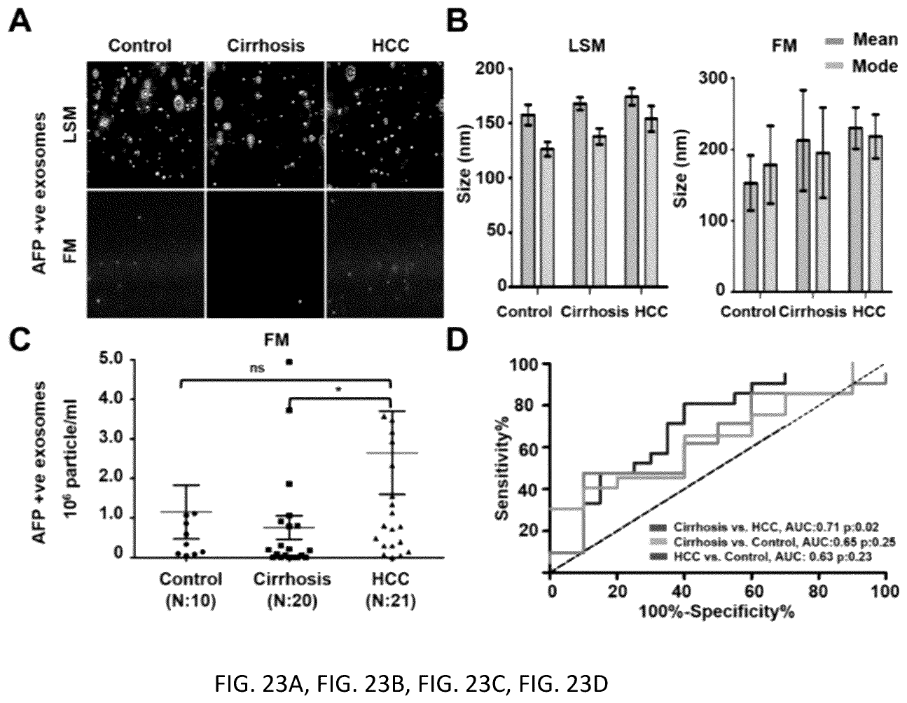

To extend the clinical utility of serum HCC exosome quantification, a serum cohort from patients with a confirmed diagnosis of no underlying liver disease (healthy normal), cirrhosis, or HCC was procured. The individual performances of AFP and GCP3 versus a combination of GPC3/AFP were investigated across the patient subgroups. Diagnostic sensitivity and specificity values for optimal cut-offs are shown in Table 4. The results of serum HCC-derived exosome quantification using GPC3 PE-conjugated antibody are shown in FIG. 22. A representative image of particles in motion in LSM and FM is shown in FIG. 22A. GPC3-positive vesicles were detected more frequently in HCC cases compared to cirrhosis and healthy controls. Negative fluorescence staining was more frequently detected in normal serum and cirrhosis samples. Analysis of GPC3-labeled exosomes in each patient subgroup revealed particles in the range of 100 to 150 nm, supporting that they are primarily exosomes. The mean and mode particle sizes captured in FM and LSM for GPC3 labeled EV samples are shown in FIG. 22B. The particles counted in FM were normalized with the total particle number in LSM to compare the distribution of GPC3-bound vesicles between normal, cirrhosis, and HCC (FIG. 22C). A statistically significant increase in GPC3+ve exosomes was detected in HCC patients compared to malignancy-free cirrhosis. A receiver operator curve was plotted to determine the diagnostic power in clinical HCC specimens. It yielded AUC values of 0.79 for discriminating HCC and cirrhotic non-HCC cases (p=0.02) (FIG. 22D). The performance of AFP antibody capturing HCC exosomes was examined using identical samples with the same analytical parameters (FIG. 23A-B). The quantification of HCC exosome captured using PE-labeled AFP antibody in normal, cirrhosis, and HCC was performed using an exact setting of LSM and FM of NTA. The AFP+ve HCC exosome number was normalized to the total particle counts obtained in LCM mode. While AFP+ve exosome concentration was significantly higher in the HCC group than in the cirrhosis group, there was no significant difference between the control and HCC groups (p=0.03 and p=0.09) (FIG. 23C). The ROC analysis showed that the AUC value of AFP+ve exosomes for detecting HCC cases was 0.71 and this was statistically significant (p=0.02). Lastly, we performed HCC exosome quantification after immunocapture of exosomes using PE-labeled GPC3 and PE-labeled AFP antibodies in the same set of samples. The HCC exosome counts were normalized with the total particle counts obtained in LCM.

TABLE 4
Optimal cut-offs of F-NTA measurements in serum
sample cohort for diagnosing HCC cases.

| Variable | Cut off (particle/ml)* | Sensitivity (%) | Specificity (%) |
|---|---|---|---|
| GPC3 | 8.02 × 10⁵ | 85 | 65 |
| AFP | 2.35 × 10⁵ | 80 | 60 |
| GPC3 + AFP | 3.22 × 10⁵ | 95 | 45 |

*Cut-off values were normalized by using total and fluorescent particle concentrations.

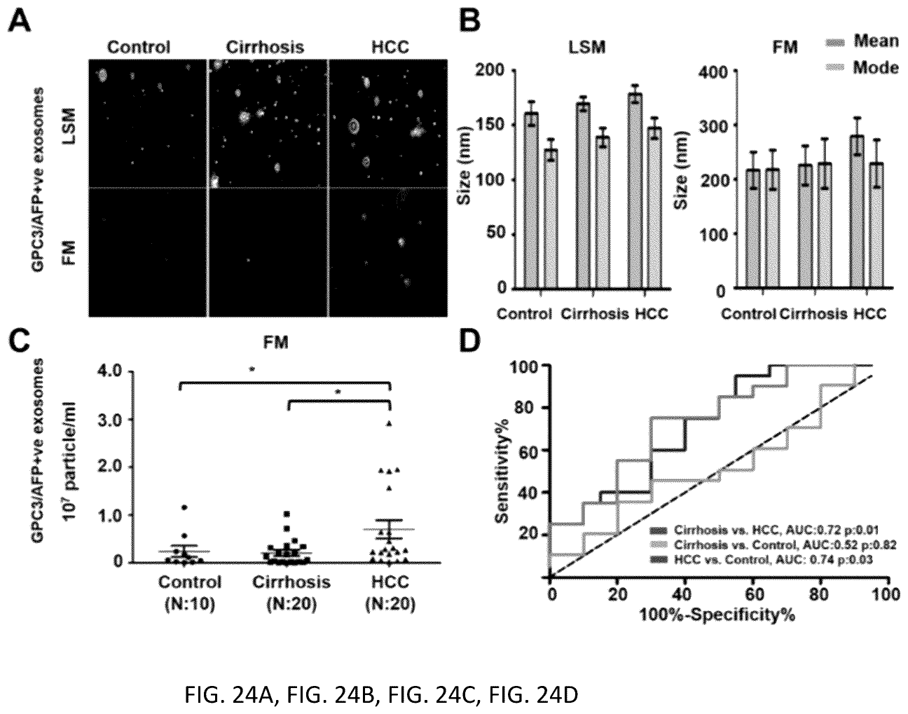

The performance of HCC exosome quantification using a combination of GPC3, and AFP antibodies together appears to be superior to AFP alone but not to GPC3 alone (FIG. 24A-D). All these results indicate that F-NTA is sensitive enough to quantify HCC exosomes using PE-labeled tumor-specific antibodies. This analysis demonstrates that the use of GPC3-PE antibody alone performed well compared to the combination of AFP and GPC3 for HCC exosome quantification.

v. Comparison of Immune Magnetic Bead Affinity Flow Assay with Direct Quantification of HCC Exosome by F-NTA:

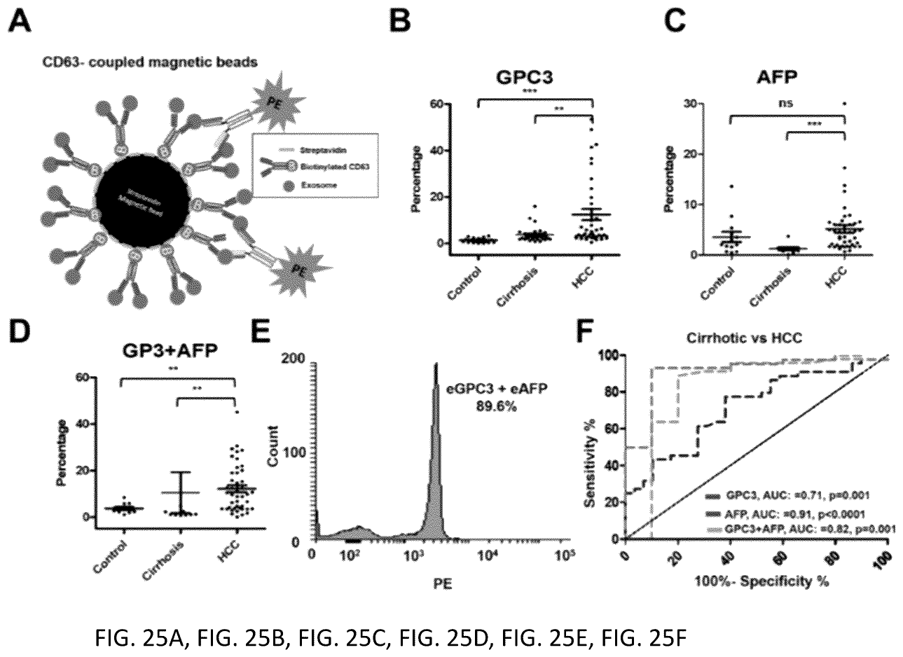

The performance of HCC exosome quantification by F-NTA in the same set of samples was compared with the magnetic bead assay. The immunomagnetic beads were prepared by mixing streptavidin-conjugated magnetic beads with biotinylated CD63 antibodies, followed by blocking and incubation directly with the serum for EV capture. This assay principle is that serum exosomes immobilized onto beads reacted either with PE-labeled AFP or PE-labeled GPC3 or in a combination of both antibodies (FIG. 25A). The magnetic bead assay was used to determine the performance of GPC3 and AFP alone and in combination markers for HCC detection using the same three groups of serum cohorts. The GPC3-positive exosomes were detected significantly higher in the serum of patients with HCC than in cirrhosis without HCC (FIG. 25B). The AFP-positive exosomes were detected considerably more in the HCC serum as compared to the control and cirrhosis (FIG. 25C). The percentage of HCC exosome detection was improved by the combination of two antibodies (FIG. 25D). The sensitivity and specificity of magnetic bead assay using single and double antibodies were determined by ROC analysis measurement (FIG. 25E). This analysis shows that the combination of AFP and GPC3 improved the sensitivity of HCC detection among cirrhotic patients, but the specificity decreased.

vi. Direct Comparison of HCC Exosome Quantification with Imaging-Based Diagnosis:

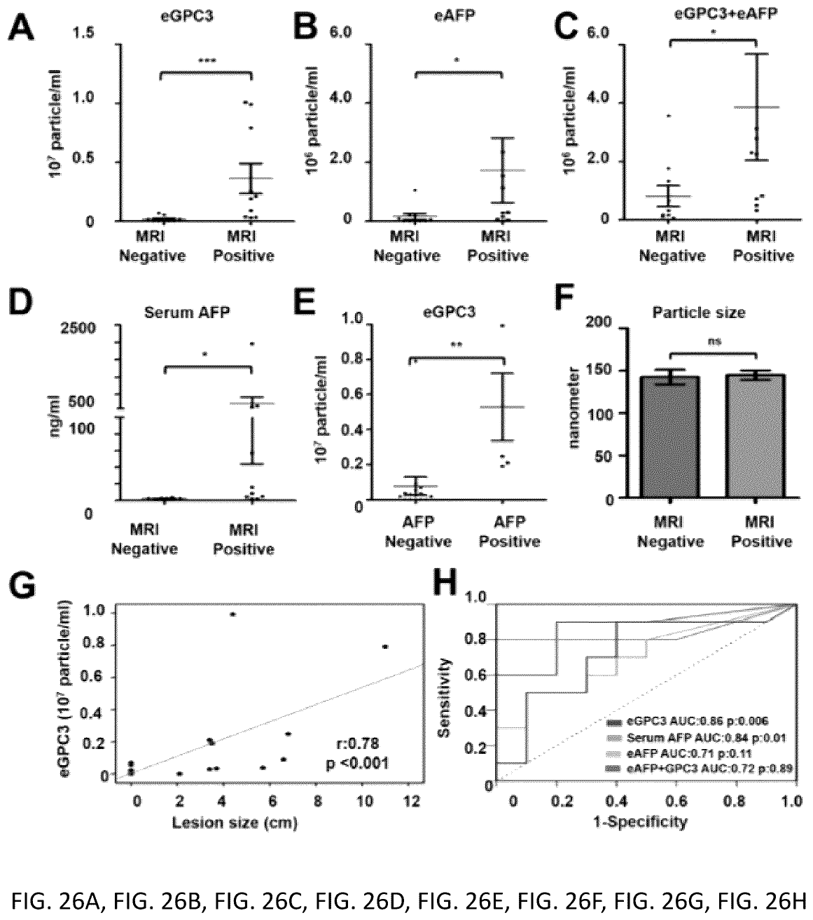

The performance of direct serum HCC-exosome quantification for liver tumor diagnosis was validated using a set of HCC whose diagnosis and tumor size were established by the radiologist through gold-standard MRI-based liver imaging. This validation study included 10-serum samples of liver cirrhosis without HCC and 10-serum samples with HCC. The demographic and clinical characteristics of these patients are summarized in Table 5. The HCC-exosome quantification after immunocapturing with antibodies to either GPC3 or AFP and in combination was performed by F-NTA using identical conditions. Normalized GPC3+ve exosome concentration was found significantly higher in MRI-confirmed HCC cases than the MRI-confirmed, HCC-free cirrhotic group (1.4×10⁶ IQR₂₅₋₇₅ 3.2×10⁵-3.8×10⁶ particle/ml vs 9.4×10⁴ IQR₂₅₋₇₅ 0-3.2×10⁵ particle/ml respectively p<0.0001) (FIG. 26A). The comparison of MRI negative and positive groups in terms of AFP+ve or GPC3/AFP+ve exosome concentration is summarized in FIG. 26B-C. Serum AFP levels were found significantly higher in MRI positive group than in MRI negative group (2.5 IQR₂₅₋₇₅ 2-3.1 ng/ml vs. 12.5 IQR₂₅₋₇₅ 3.8-148 ng/ml respectively and p=0.04) (FIG. 26 D). Despite there being no statistical direct correlation between GPC3+ve exosome concentration and serum AFP levels, GPC3+ve exosome concentration was significantly higher in the high serum AFP (>20 ng/ml) group than in the low-AFP group (p=0.034) (FIG. 26E). The size of the HCC-exosome in the MRI-positive and negative groups is comparable (FIG. 26F). There is a positive correlation between GPC3+ve HCC exosome detection by F-NTA with tumor size assessed by liver imaging (r=0.78 and p<0.001) (FIG. 26G). The ROC analysis of the MRI-positive and negative samples shows GPC3+ve exosomes separate well with AUC value: 0.86, p: 0.006 compared to AFP+ve exosomes and a combination of GPC3/AFP+ve exosomes with AUC: 0.72, p: 0.89 (FIG. 26H).

TABLE 5
Comparison of MRI negative and positive groups
for demographics, F-NTA, and serum AFP results.

| | | MRI negative | | MRI positive | | |
|---|---|---|---|---|---|---|
| Parameter | | N: 10 | | N: 10 | | p value |
| Age | (Mean ± SD) | 57.7 ± 11.7 | | 63 ± 7.8 | | 0.25 |
| Gender | | | | | | |
| Female N (%) | | 5 | (50) | 3 | (30) | 0.65 |
| Male N (%) | | 5 | (50) | 7 | (70) | |
| Etiology | | | | | | |
| HCV N (%) | | 5 | (50) | 5 | (50) | 0.87 |
| ALD N (%) | | 1 | (10) | 1 | (10) | |
| HCV + ALD N (%) | | 2 | (20) | 2 | (20) | |
| NASH N (%) | | 2 | (20) | — | | |
| Other N (%) | | — | | 2 | (20) | |
| F-NTA | | | | | | |
| GPC3 | 10⁵ particle/ml * | 0.9 | (0-3.2) | 14 | (3.2-38) | 0.005 |
| AFP | 10⁵ particle/ml * | 0.2 | (0-2) | 2.1 | (0.3-12) | 0.046 |
| GPC3/AFP | 10⁵ particle/ml * | 2.4 | (0.9-14) | 15 | (4.4-28) | 0.05 |
| Serum AFP | pg/ml | 2.5 | (2-3.1) | 12.5 | (3.8-148.5) | 0.009 |

F-NTA: Fluorescent Nanoparticle Tracking Analysis,
SD: Standard deviation,
MRI: Magnetic Resonance Imaging,
ALD: Alcoholic liver disease,
NASH: Non-alcoholic steatohepatitis,
GPC3: Glypican 3,
* Normalized values were given as median, 25th and 75th quartiles according to total exosome concentrations, Mann Whitney U test was used for comparison of groups in terms of Age, FNTA GPC3, Serum AFP.
Qi Square and Fisher's exact test was used for comparison of group distributions.

4. Discussion

Circulating EVs are relatively easy to isolate using minimally invasive procedures and offer an attractive biomarker target to monitor human health and disease progression. With no consensus on exosome isolation standards, it is proposed that the direct quantification of exosome-biomarkers represents the ideal approach for studying the biological relationship between EV signals and their source tissue at the different stages of disease progression. This study explores the direct immunocapture of HCC-specific EVs using a fluorescence-tagged antibody and then quantifying labeled EVs using F-NTA. The presented data support the F-NTA assay's excellent sensitivity and specificity for HCC exosome quantification. Using serially diluted fluorescence beads, the dynamic range of F-NTA for accurate concentration measurement of exosomes was found to be 10⁴ to 10⁸ particles/ml. Initial validation studies using serum samples revealed a high exosome concentration in human serum and therefore required volumes as low as 10 μl to fall within the F-NTA dynamic range. A critical aspect of EV quantification in serum is plasma is maintaining immunoreactivity and biological activity following isolation. For example, the high mechanical stress required by several EV isolation protocols can alter the morphology of EVs due to the vesicle tethering and interfere with their recovery and quantification. Additionally, these protocols may lead to denaturing the epitopes and diminish the immunoreactivity. The immunocapture approach used here relies on the binding of EV surface proteins with antibodies, therefore, allowing the recovery of HCC-specific EVs. This approach can be adapted for diagnostics purposes to incorporate specific protein targets for exosome capture. AFP and GPC3 are two proteins that have been widely used in HCC diagnosis. GPC3 is only produced by HCC cells, not the surrounding cirrhotic liver, whereas AFP is expressed in cirrhotic hepatocytes as well as HCC tumors. It has been shown that Huh7 cells release exosomes that express AFP and GPC3. GPC3 and AFP antibodies were selected to capture HCC exosomes. Excessive unbound fluorescence antibodies can interfere with EV quantification using F-NTA, highlighting the importance of purifying fluorescently labeled EVs from unbound fluorescent antibodies. Column purification and antibody labeling allowed the preparation of pure population PE-labeled EVs for F-NTA quantification. Column purification generated greater quantity for the direct quantification of EVs in the serum samples. Optimal antibody dilution was also critical for accurate quantification using F-NTA.

It was first assessed whether EV size distribution by F-NTA was maintained following immunocapture isolation and size exclusion column. Using PE-labeled CD9 or PE-labeled ARF6 it was shown that this method can be used to assess the distribution of exosomes and microvesicles in the serum of patients with or without advanced liver disease. Specific pathological conditions alter EVs' production and composition causing disease-specific alterations in EV composition compared to a reference healthy population. Multifaceted stress response alters autophagic mechanisms and it causes generating many membrane-enclosed lipid-bilayer vesicles to encapsulate specific cargoes of cellular plasticity. For example, induction or inhibition of autophagy leads to an increase in the release of microvesicles and exosomes during chronic viral and bacterial infection. By understanding the disease- and tissue-specific EV-associated biomarkers, this novel immunocapture assay can be utilized to accurately detect and isolate these EVs in the blood. The results show that circulating EVs are heterogenous since most cells in the body release EVs to maintain cellular homeostasis. Surprisingly, more than 50% of serum EVs did not express conventional exosome or microvesicle markers. In addition to EVs shed from infected cells and diseased tissues, many other biomolecules including protein aggregates, protein-DNA and protein-RNA complexes, ultra-fine bubbles, salt precipitates, and lipoproteins aggregates can be co-isolated from peripheral blood samples. It was examined whether the cellular origin of circulating EVs could be determined using the F-NTA approach. Quantification of hepatocyte-derived EVs was performed using a PE-labeled antibody to the asialoglycoprotein receptor (ASGPR1). ASGPR1 expression in the liver is decreased in cirrhosis and HCC due to loss of polarized secretion.

Clinical utility was also demonstrated with the F-NTA-based HCC exosome quantification approach. The ease and accuracy of the method allowed for HCC exosome quantification by combining it with immunocapture and size exclusion chromatography. Two cancer-specific antibodies were selected that are exclusively expressed on EV of HCC origin and showed minimal cross-reactivity with cholangiocarcinoma, a liver cancer of different cell origin. Our data show that the F-NTA is sensitive enough to determine the concentration of HCC exosomes in clinical samples. Using GPC3, AFP, and the biomarker combination, we showed that the detection of HCC-derived exosomes is specific to HCC diagnosis with AUC values of 0.79 for GPC3, AUC values of 0.71 for AFP, and AUC values of 0.72 to discriminate cirrhosis vs HCC. The F-NTA-based measurement correlates well with data generated by immunomagnetic bead-based flow cytometry for HCC exosome detection. To demonstrate whether this HCC-exosome quantification can be a potential serum test for detecting early-stage HCC, the assay was validated using the HCC cohort whose diagnosis was confirmed by MRI-based liver imaging. The HCC exosome quantification data correlated well with the diameter of the index HCC tumor assessed by MRI. Biostatistical analysis shows that quantification of HCC exosomes using GPC3 antibody is consistent with serum AFP levels. This indicates that the GPC3+ve exosome concentration in combination with serum AFP may improve diagnostic potential. Quantification of HCC exosomes using AFP antibody increased background staining and decreased the sensitivity and specificity of GPC3-based exosome level in identifying the HCC population. The diagnostic value of F-NTA-based HCC exosome quantification using GPC3 antibody is excellent that can be used for HCC detection among liver cirrhosis. Despite the limitation of F-NTA which is a time-consuming procedure, this analysis can be extended in clinical practice for the diagnosis of HCC with developing automation techniques.

5. Conclusions

For many years, HCC surveillance has centered around ultrasound imaging with the serum AFP biomarker. More recently, gender, age, and three serum markers (AFP-L3, AFP, and Des-carboxyprothrombin) have been combined to develop the GALAD score for HCC surveillance. Unfortunately, the GALAD score performance did not outperform other methods based on recent studies. There is a need to develop HCC biomarkers directly associated with the biology of HCC. Recently, many recent publications show that various biomolecules such as circulating tumor DNA, miRNA, non-coding RNA, and protein detection in the serum EVs have immense potential as serum biomarkers for HCC detection. Many of these candidate markers do not address the potential challenges facing the early detection of cancer, which include: (i) understanding the molecular biology of cancer etiology that contribute to cancer initiation; (ii), early identification of high-risk patients; (iii), identifying and validating organ-specific biomolecules for early-stage cancer detection; (iv), development of technology to quantify these molecules in their native state; (v), appropriate performance evaluation through multi-site collaboration. Ongoing research shows that adaptive plasticity to multifaceted stress response generated during chronic HCV infection determines who is at risk of HCC development. Biomolecules specific to liver cancer have been selected and have showm altered expression during cancer development due to impaired autophagy. This study aimed to develop an innovative assay that can rapidly and directly quantify HCC exosomes using a small quantity of serum using specific molecules amendable to biomarker development, prognosis, and early HCC detection. This assay is simple; therefore, it can be adapted to any clinical laboratory for cancer exosome quantification as a method for HCC detection in the liver through a blood test.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of treating a subject having hepatocellular carcinoma (HCC) comprising: administering an HCC therapeutic to a subject identified in need thereof, wherein the subject was identified as being in need of thereof by determining the subject had an increased number of glypican 3 (GPC3) positive exosomes in a sample obtained from the subject as compared to a control.

2. The method of claim 1, further comprising determining the subject had an increase level of alpha-fetoprotein positive exosomes.

3. The method of any one of claim 1 or 2, further comprising determining the subject was in need of an HCC therapeutic by determining that the subject had an increase level of syntenin-1 positive exosomes.

4. The method of any one of claims 1-3, further comprising isolating HCC-derived exosomes from the subject and determining whether the HCC-derived exosomes are enriched with GPC3.

5. The method of claim 4, wherein the HCC-derived exosomes are isolated from the blood or serum of the subject.

6. The method of any one of claims 1-5, further comprising determining the levels of exosome-derived GPC3 (eGPC3) in the blood or serum of the subject.

7. The method of any one of claims 1-6, wherein the method of determining the subject has an increased number of GPC3-positive exosomes or increased number of GPC3-enriched exosomes as compared to a control comprises isolating GPC3-enriched exosomes via an enzyme-linked immunosorbent assay (ELISA).

8. The method of any one of claims 1-7, wherein the sample is a plasma sample.

9. The method of any one of claims 1-8, wherein the subject has cirrhosis.

10. The method of any one of claims 1-9, wherein the HCC therapeutic is chemotherapy.

11. The method of any one of claims 1-10, wherein the control is a subject having cirrhotic liver and not HCC.

12. The method of any one of claims 1-11, wherein the increase in number of GPC3 positive exosomes is at least 2-fold that of the control.

13. The method of any one of claims 1-12, further comprising wherein the subject was identified as being in need of thereof by determining the level of alpha-fetoprotein positive exosomes.

14. The method of any one of claims 1-13, further comprising wherein the subject was identified as being in need of thereof by determining the level of syntenin-1 positive exosomes.

15. A method of diagnosing and treating a subject comprising
a. detecting whether the number of GPC3 positive exosomes is increased in the subject;
b. diagnosing the subject with HCC when the presence of elevated GPC3 positive exosomes is detected; and
c. administering a therapeutically effective amount of an HCC therapeutic to the subject.

16. The method of claim 15, wherein the increased number of GPC3 positive exosomes is detected in a sample of the subject.

17. The method of claims 15-16, wherein the sample is a plasma sample.

18. The method of claims 15-17, wherein the increased GPC3 expression is exosome derived.

19. The method of claims 15-18, wherein the HCC therapeutic is chemotherapy.

20. The method of claims 15-20, wherein a subject having increased number of GPC3 positive exosomes has an increase compared to a control.

21. The method of claims 15-20, wherein the subject has cirrhosis.

22. The method of claims 15-21, wherein the control is a subject having cirrhotic liver and not HCC.

23. The method of claims 15-22, wherein the increase is at least 2 fold that of the control.

24. The method of claims 15-23, further comprising an increase in p62 expression.

25. A method of diagnosing a subject as having HCC comprising
a. detecting whether the number of GPC3 positive exosomes is increased in the subject; and
b. diagnosing the subject with HCC when the presence of elevated GPC3 positive exosomes is detected.

26. The method of any one of claims 1-25, wherein the GPC3 expression is detected using immune-affinity flow cytometry.

27. A method of detecting hepatocellular carcinoma (HCC) in a subject comprising:
determining the number of GPC3 positive exosomes in a sample obtained from the subject; and
comparing the number of GPC3 positive exosomes from the subject to a control;
wherein an increase in the number of exosomes in the sample, as compared to the control, detect HCC in the subject.

28. A method of screening comprising
a. detecting the presence of GPC3 positive exosomes in a sample from a subject having HCC;
b. adding a therapeutic to the sample;
c. detecting the presence of GPC3 positive exosomes in the sample after incubating with the therapeutic;
d. determining the therapeutic treats HCC when a decrease in GPC3 positive exosomes are present in the sample after administering the therapeutic.

29. A method of screening a whether a subject having HCC is responsive to a therapeutic comprising
a. detecting the presence of GPC3 positive exosomes in a sample from the subject;
b. administering the therapeutic to the subject;
c. detecting the presence of GPC3 positive exosomes in a sample taken after administering the therapeutic;
d. determining whether the subject is responsive to the therapeutic,
wherein a decrease in GPC3 positive exosomes in the sample taken after administering the therapeutic indicates the subject is responsive to the therapeutic.
